(19) United States
(12) Patent Application Publication
(10) Pub. No.: US 2007/0093520 A1
(43) Pub. Date: Apr. 26, 2007

(54) Method of treatment of diarrhea-predominant irritable bowel syndrome in a subject

(76) Inventor: Steven David Caras, Marietta, GA (US)

Correspondence Address: MAYER, BROWN, ROWE & MAW LLP, 1909 K STREET, N.W., WASHINGTON, DC, 20006, US

(21) Appl. No.: 11/405,036

(22) Filed: Apr. 17, 2006

Related U.S. Application Data
(60) Provisional application No. 60/671,488, filed on Apr. 15, 2005.
(60) Provisional application No. 60/672,531, filed on Apr. 19, 2005.
(60) Provisional application No. 60/672,524, filed on Apr. 19, 2005.

(51) Int. Cl. A61K 31/4745

(52) U.S. Cl. 514/291

(57) ABSTRACT

A method of treatment of diarrhea-predominant IBS in a subject, comprising administering to a subject in need of treatment a therapeutically effective amount of a composition comprising cilansetron or a pharmaceutically acceptable derivative thereof; and providing information that indicates that constipation, abdominal pain, upper respiratory tract infection and/or nausea may occur after administering the composition.

### CROSS REFERENCE TO RELATED APPLICATIONS

This application claims the benefit of U.S. Provisional Application Ser. Nos. 60/671,488, filed on Apr. 15, 2005, 60/672,531, filed on Apr. 19, 2005, and 60/672,524, filed on Apr. 19, 2005. These applications, in their entirety, are incorporated herein by reference.

### FIELD OF THE INVENTION

The invention relates to the use of cilansetron and, more particularly, to a method for treating diarrhea-predominant irritable bowel syndrome in a subject.

### BACKGROUND OF THE INVENTION

Irritable bowel syndrome (IBS) affects approximately 10-20% of the general population. It is the most common disease diagnosed by gastroenterologists and one of the most common disorders seen by primary care physicians.

Despite the high incidence of IBS among men and women, however, treatments for IBS and for diarrhea-predominant and non-constipated forms of IBS are only partially effective in providing adequate safe, long-term symptom relief to patients.

Accordingly, there remains a need for improved methods of treatment of IBS in patients in need thereof.

### SUMMARY OF THE INVENTION

This invention provides a method of treatment of diarrhea-predominant IBS in a subject, comprising administering to a subject in need of treatment a therapeutically effective amount of a composition comprising cilansetron or a pharmaceutically acceptable derivative thereof; and providing information that indicates that constipation, abdominal pain, upper respiratory tract infection and/or nausea may occur after administering the composition.

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and providing information that indicates that angina pectoris and/or a cardiac arrhythmia may occur after administering the composition.

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and providing to the subject dosage, administration and adverse reaction information pertaining to the composition, wherein the adverse reaction information comprises information that indicates that constipation, abdominal pain, upper respiratory tract infection and/or nausea may occur after administering, the composition.

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and providing to the subject dosage, administration and adverse reaction information pertaining to the composition, wherein the adverse reaction information comprises information that indicates that angina pectoris and/or a cardiac arrhythmia may occur after administering the composition.

In another aspect, the invention provides a method of treatment of diarrhea-predominant IBS in a female subject, comprising: administering to a subject in need of treatment a therapeutically effective amount of a composition comprising cilansetron or a pharmaceutically acceptable derivative thereof; and providing information that indicates that constipation, diarrhea, sinusitis, and/or urinary tract infection may occur after administering the composition.

In another aspect, the invention provides a method of treatment of diarrhea-predominant IBS in a male subject, comprising: administering to a subject in need of treatment a therapeutically effective amount of a composition comprising cilansetron or a pharmaceutically acceptable derivative thereof; and providing information that indicates that an increase in blood creatinine phosphokinase may occur after administering the composition.

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and monitoring the subject for a treatment-emergent adverse event selected from constipation, abdominal pain, nausea, upper respiratory tract infection, urinary tract infection, rectal hemorrhage, angina pectoris, a cardiac arrhythmia, sinusitis, fecal occult blood, nasopharyngitis, and/or an increase in blood creatinine phosphokinase.

In another aspect, the invention provides a method for safe long-term treatment of diarrhea-predominant IBS in a subject, comprising administering a composition comprising cilansetron or a pharmaceutically acceptable derivative thereof to a subject in need of treatment for a period of at least about 52 weeks.

In another aspect, the invention provides a method of treatment of diarrhea-predominant IBS in a subject, comprising administering a composition comprising cilansetron or a pharmaceutically acceptable derivative thereof to a subject in need of treatment in an amount sufficient to substantially provide relief of at least one symptom associated with diarrhea-predominant IBS in the subject for a period of at least about 52 weeks.

In another aspect, the invention provides a method of improving quality of life in a subject having diarrhea-predominant IBS, comprising administering a composition comprising cilansetron or a pharmaceutically acceptable derivative thereof to a subject in need of treatment in an amount sufficient to substantially improve quality of life in the subject for a period of at least about 52 weeks.

In another aspect, the invention provides a method of treatment of diarrhea-predominant IBS in a male subject, comprising administering a composition comprising cilansetron or a pharmaceutically acceptable derivative thereof in an amount sufficient to substantially maintain a prevalence of treatment-emergent constipation of less than about 15% across a statistically significant population of male subjects for a period of at least about 52 weeks.

These and other aspects of the present invention are describe more fully herein below.

### BRIEF DESCRIPTION OF THE DRAWINGS

FIG. 1 is a schematic of the design of the 3007 study.

FIG. 2 is a plot of mean change of quality of life subscale scores from baseline to endpoint of treatment of subjects with cilansetron or placebo.

### DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

It is to be understood that any ranges, ratios and ranges of ratios that can be formed by any of the numbers or data present herein represent further embodiments of the present invention. This includes ranges that can be formed that do or do not include a finite upper and/or lower boundary. For example, by way of illustration and not limitation, referring to FIG. 38, wherein it is illustrated that the difference in mean plasma concentration of EE between OC+cilansetron and OC administration alone is less than about 10 pg/mL or even less than about 1 pg/mL. Accordingly, the skilled person will appreciate that such ratios, ranges and values are unambiguously derivable from the data presented herein.

The following acronyms are used herein: 5-ASA )5-Aminosalicylic acid); 5-HT (5-Hydroxytryptamine); β-HCG (Beta Human Chorionic Gonadotrophin); ACE (angiotensin-converting enzyme); AE (Adverse Event); ALT (SGPT) (Alanine Aminotransferase); AP (Alkaline Phosphatase); AST (SGOT) (Aspartate Aminotransferase); ATC (Anatomical Therapeutic Chemical); AUC (Area Under the Curve); BMI (Body Mass Index); bpm (Beats per Minute); BUN (Blood Urea Nitrogen); B.V. (Besloten Vennootschap (Incorporated; Limited)); C/C (Subjects who were randomized to cilansetron 2 mg TID in Study 3006 and continued cilansetron 2 mg TID during Study 3007); CK (Creatine Kinase, Creatinine Phosphokinase); Cmax (Peak Plasma Concentration from the Measured Data); CRF (Case Report Form); ECG (Electrocardiogram); FDA (Food and Drug Administration); GCP (Good clinical practice); GERB (Gastrointestinal Events Review Board); GGT (Gamma-Glutamyltransferase); GI (Gastrointestinal); HEENT (Head, Ears, Eyes, Nose, Throat); IBS (Irritable Bowel Syndrome); IRB (Institutional Review Board); LAD (Left axis deviation); LDH (Lactic Dehydrogenase); MedDRA (Medical Dictionary for Regulatory Activities); NA (Not Applicable); NEC (Not Elsewhere Classified); NOS (Not Otherwise Specified); NSAID (non-steroidal anti-inflammatory drug); P/C (Subjects who were randomized to placebo in Study 3006 and were taking cilansetron 2 mg TID during Study 3007); PEG (polyethylene glycol); PT (Prothrombin time); QOL (Quality of Life); QT_{c}(B) (Corrected QT interval (Bazett)); QT_{c}(F) (Corrected QT interval (Fridericia)); RBC (Red Blood Cell); SAE (Serious Adverse Event); SE (Standard Error); SSRI (Selective serotonin reuptake inhibitor); TEAE (Treatment-Emergent Adverse Event); TID (Three Times per Day); TSH (Thyroid stimulating hormone); WBC (White Blood Cell); and WHO (World Health Organization).

“Cilansetron”, as used herein, is understood to refer to (R)-(−)-4,5,6,8,9,10-hexahydro-10-[(2-methyl-1H-imidazol-1-yl)methyl]-11H-pyrido-[3,2,1-jk]-carbazol-11-one (alternative name: (10R)-5,6,9,10-tetrahydro-10-[(2-methyl-1H-imidazol-1-yl)methyl]-4H-pyrido[3,2,1-jk]carbazol-11(8H)-one), which is disclosed, for example, in U.S. Pat. No. 6,566,369, the contents of which are incorporated herein by reference.

Cilansetron can be administered in any suitable dose, and using any suitable dosing schedule. The dosage of cilansetron administered according to the methods of the present invention may be, for example, from about 0.1 mg to about 40 mg daily, such as from about 1 mg to about 38 mg daily, from about 2 mg to about 36 mg daily, from about 2 mg to about 34 mg daily, from about 2 mg to about 32 mg daily, from about 2 mg to about 30 mg daily, from about 2 mg to about 28 mg daily, from about 2 mg to about 26 mg daily, from about 2 mg to about 24 mg daily, from about 2 mg to about 22 mg daily, from about 2 mg to about 20 mg daily, from about 2 mg to about 18 mg daily, from about 2 mg to about 16 mg daily, from about 2 mg to about 14 mg daily, from about 2 mg to about 12 mg daily, from about 2 mg to about 10 mg daily, from about 2 mg to about 8 mg daily (such as or from about 4 mg to about 8 mg daily, from about 2 mg to about 6 mg daily, or from about 2 mg to about 4 mg daily. Moreover, cilansetron may be administered one or more times a day, such as two or more, three or more, four or more, five or more, or even six or more times daily. In a preferred embodiment, 2 mg cilansetron is administered three times daily (TID). Moreover, cilansetron can be administered on alternate days or on consecutive days. Cilansetron can also be administered in any suitable formulation such as, for example, a tablet, capsule, gelcap, or solution (e.g., injectable or inhalable solution). In one embodiment, cilansetron is in the form of a 2 mg, 4 mg or 8 mg capsule. In another embodiment, cilansetron is in the form of a 4 mg/2 mL or 8 mg/4 mL solution. In another embodiment, cilansetron is in the form of a 1 mg, 2 mg, 4 mg, 8 mg, 16 mg or 32 mg tablet.

Cilansetron may be administered in the form of a any pharmacologically acceptable acid addition salts, as described for example in U.S. Pat. No. 6,566,369 (the entire contents of which are incorporated herein by reference. In one embodiment, cilansetron is administered in the form of cilansetron hydrochloride or cilansetron hydrochloride monohydrate. Additionally, cilansetron can be administered in any form and in combination with any known diluent, filler, salt, buffer, stabilizer, solubilizer, lipid, or other material, as disclosed, for example in the '369 patent. In one embodiment, cilansetron is administered in a composition comprising: 4 parts cilansetron hydrochloride monohydrate, 30 parts corn starch, 70 parts lactose, 5 parts Kollidon 25®, 2 parts magnesium stearate, and 3 parts talcum, as described, for example in U.S. Pat. No. 6,566,369 (the entire contents of which are incorporated herein by reference). In an embodiment, cilansetron is administered in a 2 mg film-coated tablet for oral use in humans, comprising

(i) about 1.5 mg to about 3 mg cilansetron.HCl.H₂0,

(ii) about 40 mg to about 60 mg corn starch,

(iii) about 70 mg to about 100 mg mannitol,

(iv) about 3 mg to about 7 mg povidone,

(v) about 0.05 mg to about 1 mg citric acid monohydrate,

(vi) about 1 mg to about 5 mg crospovidone,

(vii) about 0.05 mg to about 2 mg colloidal silica, and

(viii) about 1 mg to about 3 mg stearic acid.

In another embodiment, cilansetron is administered in a 2 mg film-coated tablet for oral use in humans, comprising: 2.34 mg cilansetron.HCl.H₂0, 51.78 mg maize starch, 84.48 mg mannitol; 4.90 mg povidone; 0.50 mg citric acid monohydrate; 3.0 5 mg crospovidone; 1.0 mg colloidal anhydrous silica; 2.0 mg stearic acid; and a coating containing Opadry® 03B28686 (white) or Opadry® Y-1-7000 (white).

This invention provides a method of treatment of diarrhea-predominant IBS in a subject, comprising administering to a subject in need of treatment a therapeutically effective amount of a composition comprising cilansetron or a pharmaceutically acceptable derivative thereof; and providing information that indicates that constipation, abdominal pain, upper respiratory tract infection and/or nausea may occur after administering the composition. As used here, “administer” is defined to include administering (e.g., giving, selling or providing) of the composition to a subject or group of subjects by a medical professional or other third person(s), as well as self-administering of the composition by the subject. The information provided to the subject can comprise any suitable information indicating that a specific treatment-emergent adverse event(s) may occur following administering of the composition. In particular, for example, the information provided may comprise information indicating that at least one symptom of one or more of the following adverse events can occur after administering of the composition: constipation, abdominal pain/discomfort, nausea, upper respiratory tract infection, urinary tract infection, lower respiratory tract and lung infections, rectal hemorrhage, angina pectoris, cardiac arrhythmia, sinusitis, fecal occult blood, vomiting, fecal impaction, headache, nasopharyngitis, and/or an increase in blood creatinine phosphokinase.

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and providing information that indicates that angina pectoris and/or a cardiac arrhythmia may occur after administering the composition. Cardiac arrhythmias or dysrhythmia, in this regard, can be any condition or disorder in which cardiac muscle contractions are faster, slower and/or more irregular than normal. In particular, for example, cardiac arrhythmias include bradycardia (e.g., sinus bradycardia) and tachycardia (e.g., ventricular tachycardia), as well as any other electrocardiogram (ECG) abnormalities, such as, for example, poor R-wave progression, early repolarization-normal variant, non-specific T-wave changes, borderline LA enlargement, early R-wave transition, abnormal QT_{c}(B), abnormal QT_{c}(F), prolonged QT interval, and/or effects associated therewith (e.g., palpitations and/or syncope).

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and providing to the subject dosage, administration and adverse reaction information pertaining to the composition, wherein the adverse reaction information comprises information that indicates that constipation, abdominal pain, upper respiratory tract infection and/or nausea may occur after administering the composition. The provided information, in this regard, can further comprise any suitable information that aids in the safe and effective administering of the composition to a subject or group (e.g., statistically significant population) of subjects, such as any information that is required to be included in package insert materials by the Food & Drug Administration with approved pharmaceuticals. In particular, for example, the provided information can comprise any suitable prescribing information, such as, for example, efficacy information, warnings, indication and contraindication information (e.g., information indicating that the composition is contraindicated for subjects having constipation-predominant IBS), as well as information indicating that immediate hypersensitivity reactions to the composition may occur after administration of the composition. As used herein, “a statistically significant population” is understood to refer to two or more, three or more, four or more, five or more, ten or more, twenty or more, forty or more, sixty or more, eighty or more, or even one hundred or more persons.

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and providing to the subject dosage, administration and adverse reaction information pertaining to the composition, wherein the adverse reaction information comprises information that indicates that angina pectoris and/or a cardiac arrhythmia may occur after administering the composition.

In another aspect, the invention provides a method of treatment of diarrhea-predominant IBS in a subject (e.g., male or female subject), comprising administering to a subject in need of treatment a therapeutically effective amount of a composition comprising cilansetron or a pharmaceutically acceptable derivative thereof; and providing information that indicates that constipation, diarrhea, sinusitis, and/or urinary tract infection may occur after administering the composition.

In another aspect, the invention provides a method of treatment of diarrhea-predominant IBS in a subject (e.g., male or female subject), comprising administering to a subject in need of treatment a therapeutically effective amount of a composition comprising cilansetron or a pharmaceutically acceptable derivative thereof; and providing information that indicates that an increase in blood creatinine phosphokinase may occur after administering the composition. Moreover, the provided information can comprise information that an increase in blood creatinine phosphokinase occurs in less than about 25% (e.g., less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%) of subjects in a statistically significant population of subjects.

In another aspect, the invention provides a method of treatment of diarrhea-predominant IBS in a subject, comprising administering to a subject in need of treatment a therapeutically effective amount of a composition comprising cilansetron or a pharmaceutically acceptable derivative thereof; and monitoring the subject for a treatment-emergent adverse event selected from constipation, abdominal pain, nausea, upper respiratory tract infection, urinary tract infection, rectal hemorrhage, angina pectoris, a cardiac arrhythmia, sinusitis, fecal occult blood, nasopharyngitis, and/or an increase in blood creatinine phosphokinase. In this regard, if at least one treatment-emergent adverse event is detected, the dosage, dosing regimen or schedule, route of administration, and/or form of the composition administered can be altered (e.g., increased, decreased, ceased, or changed) to a dosage type or form that does not produce the at least one treatment-emergent adverse event. In this regard, for example, if at least one treatment-emergent adverse event is detected, administration of the composition can be ceased (either temporarily or permanently). “Monitoring”, in this regard, can be carried out in any suitable manner, such as, for example, via self-monitoring by a subject, via examination by a medical professional or other third person, via analysis of survey, questionnaire and/or telephonic or electronic entries, and/or through use of medical devices.

In another aspect, the invention provides a method for safe long-term treatment of diarrhea-predominant IBS in a subject, comprising administering a composition comprising cilansetron or a pharmaceutically acceptable derivative thereof to a subject in need of treatment for a period of at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, or even at least about 104 weeks. “Safe” treatment can be defined in any suitable manner, such as, for example, an incidence across a statistically significant population of subjects of any treatment-emergent serious adverse event (as defined below) of less than about 10%, less than about 9%, less than about 8%, less than about 7%, less than about 6%, less than about 5%, less than about 4%, less than about 3%, less than about 2%, or even less than about 1%.

In another aspect, the invention provides a method of treatment of diarrhea-predominant IBS in a subject, comprising administering a composition comprising cilansetron or a pharmaceutically acceptable derivative thereof to a subject in need of treatment in an amount sufficient to substantially provide relief of at least one symptom associated with IBS-D in the subject for a period of at least about 52 weeks. Non-limiting examples of symptoms of IBS-D to be relieved include stool consistency, stool frequency, urgency, feelings of incomplete evacuation, feelings of bloating, abdominal pain/discomfort, loose stools, diarrhea and/or swollen or bloated abdomen. “Adequate relief”, as described further below, is understood by those of skill in the art to mean any suitable symptom relief for a subject.

In another aspect, the invention provides method of improving quality of life in a subject having diarrhea-predominant IBS, comprising administering a composition comprising cilansetron or a pharmaceutically acceptable derivative thereof to a subject in need of treatment in an amount sufficient to substantially improve quality of life in the subject for a period of at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, or even at least about 104 weeks. Non-limiting examples of improvements in quality of life include decreasing interruption in daily activities, enhancing body image, decreasing food avoidance, enhancing interpersonal relationships, enhancing sexual performance capacity, improving social functioning, improving physical functioning, improving general health, improving vitality, enhancing social functioning, and improving mental health.

In another aspect, the invention provides a method of treatment of diarrhea-predominant IBS in a male subject, comprising administering a composition comprising cilansetron or a pharmaceutically acceptable derivative thereof in an amount sufficient to substantially maintain a prevalence of treatment-emergent constipation of less than about 30% (e.g., less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or even less than about 1%) across a statistically significant population of male subjects for a period of at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, or even at least about 104 weeks. As used herein, “a statistically significant population” is understood to refer to two or more, three or more, four or more, five or more, ten or more, twenty or more, forty or more, sixty or more, eighty or more, or even one hundred or more persons.

Clinical test data (set forth in the examples below) prove the surprising suitability of cilansetron for the treatment of IBS-D in a subject.

### EXAMPLES

Three studies (hereinafter “the 3007 Study”, “the 3008 Study” and “the 5050 Study”) were undertaken to investigate the safety and tolerability of cilansetron 2 mg TID in diarrhea-predominant IBS subjects during a 52-week treatment period, as well as the long-term effects of cilansetron on quality of life of subjects. The duration of 52 weeks was used to collect sufficient long-term safety data on cilansetron 2 mg TID.

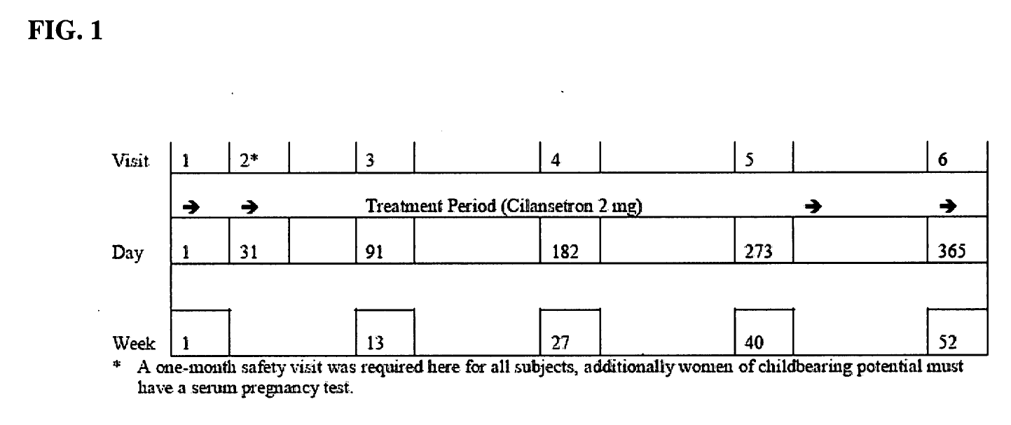

The 3007 Study was an open-label, multi-center study to investigate the safety of cilansetron 2 three times a day (TID) in diarrhea-predominant IBS subjects during a 52-week treatment period. FIG. 1 provides a schematic of the overall study design for the 3007 Study. Subjects who completed treatment under a cilansetron double-blind efficacy protocol (that preceded the 52-week 3007 study) (hereinafter “the 3006 Study”) could begin the 52-week extension on the last day of the 3006 study. All subjects received 2 mg of cilansetron TID. Subjects scheduled study visits approximately every three months during the 52-week treatment period. Since some subjects entering the study had been on placebo in the 3006 Study (which preceded the 3007 Study) and, therefore, were first time users of cilansetron, a one month interim safety visit (Visit 2) was required for all subjects entering the 3007 study. At each scheduled visit, interim assessments were performed and quantitative serum pregnancy tests were performed on female subjects of childbearing potential.

The 3008 Study was an open-label, multi-center study to investigate the safety of cilansetron 2 mg TID in diarrhea-predominant IBS subjects during a 52-week treatment period. The study was performed in approximately 150 study centers. Approximately 1600 eligible subjects were to be given cilansetron 2 mg TID. At Visit 1, pre-treatment assessments were conducted. A maximum of 14 days and a minimum of three days (due to the occult blood assessment) were allowed between Visit 1 (pre-treatment) and Visit 2 (baseline assessment). Treatment started at Visit 2, after all baseline assessment results had been reviewed and the subject had been found eligible (Day 1). Subjects were to return to the study site one month later for Visit 3 (Day 31) for their first interim assessment. After Visit 3, subjects were to return to the study site for interim assessments at Visits 4, 5, and 6 on Days 91, 182, and 273, respectively. After Visit 6 (Day 273), subjects were to return to the study site approximately three months later to finish the study with a post study assessment at Visit 7 on Day 365. All subjects who discontinued from the study were to have the same post-study assessments required at Visit 7 performed at their early termination visit.

The 5050 Study was a double-blind, randomized, placebo-controlled study to evaluate the long-term safety and tolerability of cilansetron 2 mg taken on an as needed (prn) basis for 1 year by male and female subjects with ROME II defined IBS-D. The study was performed in 68 centers. Patients were randomized in a 3:1 ratio to receive either cilansetron 2 mg prn or placebo, up to 3 times a day. Safety parameters were explored by comparing the double-blind groups, average daily doses and treatment exposure. Patient visits were scheduled as follows: Visit 1 (pretreatment), Visit 2 (Day 1), Visit 3 (Month 1), Visit 4 (Month 3), Visit 5 (Month 6), Visit 6 (Month 9), and Visit 7 (Month 12). Safety assessments included: adverse events (AEs)/serious adverse events (SAES); laboratory parameters (pretreatment visit and at Months 1, 6, and 12); electrocardiograms (pretreatment visit and at Months 1, 6, and 12); vital signs (at each visit); physical examinations and body weight (pretreatment visit and at Month 12); prior and concomitant medication use (each visit and/or through spontaneous reporting); stool cards for occult blood (every visit except Visit 1). The number of tablets taken per day was recorded daily through a validated interactive voice response system. Patients who met the ROME criteria for IBS and ROME II criteria for the diarrhea predominant subpopulation were included in the study. Patients with acute colitis of any etiology, history of chronic colitis of any etiology, or a history of intestinal obstruction, stricture, toxic megacolon, gastrointestinal (GI) perforation, or fecal impaction were excluded from the study.

Selection of Study Population—3007 Study

The 3007 Study was restricted to subjects having severe IBS defined by symptoms which were described by-the-subject as being “very often” (greater than 50% of the time), and which interfered with the subject's ability to effectively perform his or her regular activities (e.g., work, school, recreation, household, social, or travel activities).

Inclusion Criteria
1. Signed informed consent form;
2. Established diarrhea-predominant IBS subject as defined in the 3006 Study (which preceded the 3007 study);
3. Completed the 3006 Study and in the judgment of the Principal Investigator and the subjects that the continuation of treatment could benefit the subject;
4. Women of child-bearing potential had to agree to continue using a medically acceptable method of birth control or had to agree to abstain from sexual intercourse (abstinence option only with agreement from the local IRB) throughout the study and for 30 days immediately after the last dose of study drug. Medically acceptable methods of birth control were defined as either a bilateral tubal ligation or the use of either a contraceptive implant, a contraceptive injection (Depo-Provera), an intrauterine device, or an oral contraceptive that had been taken for at least three months, and that the subject agreed to continue to use during the study, or a double-barrier method, which had to consist of a combination of any two of the following: diaphragm, cervical cap, condom, or spermicide.

Exclusion Criteria
1. Evidence of severe cardiovascular, respiratory, urogenital, GI/hepatic, hematologic/immunologic, head, ears, eyes, nose, throat (HEENT), dermatologic/connective tissue, musculoskeletal, metabolic/nutritional, endocrine, neurologic/psychiatric, allergy, major surgery or other relevant diseases as revealed by history, physical examination and laboratory assessments which, in the opinion of the Investigator could interfere with the administration or assessment of study medication. This was to be reconfirmed by the medical examination performed at the normal end of study (termination) visit of the double-blind efficacy protocol prior to entering the extension study (at Visit 1).
2. Subjects who terminated for reasons other than “normal” from the double-blind efficacy protocol.
3. Females of childbearing potential who did not agree to continue using a medically acceptable method of birth control or to remain abstinent from sexual intercourse (abstinence option only with agreement from the local IRB) throughout the study or who desired to become pregnant during the course of the study and for thirty days immediately after the last dose of study drug.
4. Females of childbearing potential with an indeterminate pregnancy test at the last visit (normal end of study visit) of the double-blind efficacy protocol, could not enter into the extension study if they were found to be pregnant based on subsequent testing.
5. Subjects with a history of seizure(s).
   Removal of Patients from Therapy or Assessment

Subjects had completed this study when they had taken the study drug within the whole treatment period of 52 weeks, completed the end of study assessment, and had not withdrawn for any reason.

All subjects were free to withdraw from participation in this study at any time, for any reason, specified or unspecified, and without penalty or loss of benefits to which the subject was otherwise entitled.

Drop-Outs

Dropouts were those subjects who left the study earlier than planned for whatever reason. Dropouts were not replaced.

Data from dropouts were included in the statistical analysis. The CRF had to be completed up to the time of dropout. All dropouts after the first intake of study medication were to be given a post-study assessment as appropriate. The study termination and final comments in the CRF were to be completed for all dropouts.

Withdrawals

Withdrawals were those dropouts who were discontinued from the study for any of the following reasons:
Adverse events that were intolerable to the subject and/or in the view of the Investigator jeopardized the health of the subject;
Lack of efficacy;
Any other medical reason;
Insufficient compliance regarding time schedules or medication intake;
Protocol violation(s) that in the Investigator's was (were) incompatible with further participation of the subject in the study.
   Selection of Study Population—3008 Study

This section describes the criteria used to check the subjects eligibility for enrollment in this study. If the investigator believed that it was medically justified to have (a) minor deviation(s) from one (or more) of the eligibility criteria, and if this (these) minor deviation(s) had (have) no impact on the character of the study, a waiver could be granted for this (these) deviation(s).

Inclusion Criteria

1. Signed informed consent form;

2. Established diarrhea-predominant IBS subject as defined below;

3. Legal consenting age;

4. Weight (body mass index [BMI]) 10:

Lower limit: 18 kg/m2

Upper limit: 35 kg/m2

5. Females of child bearing potential had to have a serum pregnancy test during pre-treatment and it had to be negative (also see Section 5.5.1, Table 2) or the subject had to be surgically sterile (bilateral oophorectomy and/or hysterectomy) or at least one year postmenopausal as judged by the Investigator.

6. Women of child-bearing potential had to be using a medically acceptable method of birth control or had to agree to abstain from sexual intercourse (abstinence option only with agreement from the local EC/IRB) throughout the study and for 30 days immediately after the last dose of study drug. Medically acceptable methods of birth control were defined as either a bilateral tubal ligation or the use of either a contraceptive implant, a contraceptive injection (Depo-Provera), an intrauterine device, or an oral contraceptive that had been taken for at least three months, and that the subject agreed to continue to use during the study, or a double-barrier method, which had to consist of a combination of any two of the following: diaphragm, cervical cap, condom, or spermicide.

IBS Definition

The definition of IBS was based on the Rome criteria published in 1988, revised in 1990 and revised a second time in 1992.11

The symptoms of IBS had to include at least six months continuous or recurrent symptoms of:

7. Abdominal pain or discomfort that was:
relieved by defecation;
and/or associated with a change in frequency of stool;
and/or associated with a change of consistency of stool;
AND

8. Two or more of the following, at least a quarter of occasions or days (25%):
altered stool frequency (>3 bowel movements/day or <3 bowel movements/week);
altered stool form (lumpy/hard or loose/watery stools);
altered stool passage (straining, urgency, or feeling of incomplete evacuation);
passage of mucus;
bloating or feeling of abdominal distention.

Established IBS patients met at least one of the following criteria:
Were well known in the Investigator□s practice and diagnosed with IBS at least six months before entering the study (Visit 1);
Had been seen by another physician outside the research site and had been diagnosed with IBS at least six months prior to entering the study (Visit 1). Source documentation of the patient's IBS diagnosis, history and work-up was to be obtained from the patient's physician. A consultation with this physician was to be performed and documented. The Investigator had to corroborate this diagnosis based on the patient's medical records and a clinical evaluation.
Had been seen by another physician outside the research site and had at least a six-month history of bowel signs and symptoms that could be consistent with IBS. Source documentation substantiating the patient's bowel symptoms was to be obtained from the patient's physician. In this case, the Investigator in consultation with the patient's physician could make an IBS diagnosis after performing a clinical evaluation and excluding the appropriate medical conditions.
   Definition of Diarrhea—Predominant IBS:

The definition of diarrhea-predominant IBS was based on the Rome II criteria. Subjects were to answer the following questions:

A. In the last six months, have you had more than three bowel movements each day?

B. In the last six months, have you had fewer than three bowel movements each week?

C. In the last six months, have you had lumpy or hard bowel movements (stools)?

D. In the last six months, have you had loose, mushy or watery bowel movements (stools) ?

E. In the last six months, have you needed to strain a lot to have a bowel movement?

F. In the last six months, have you experienced an urgent need to have a bowel movement that made you rush to a toilet?

Subjects were to rate these questions according to the following scale:

0. Not at all or rarely

1. Occasionally (more than one-tenth of the time)

2. Often (more than one-quarter of the time)

3. Very often (more than one-half of the time)

4. Almost always

Subjects were included if they rated the following questions according to the above rating scale:

Rule 1:
One or more of question A, question D, or question F rated 2 or above AND
Question B, question C, and question E rated 0 or 1,

OR alternatively,

Rule 2:
Two or more of question A, question D, or question F rated 2 or above AND
One of question B or question E rated 2 or above AND
Question C with a rating of 0.

General Exclusion Criteria

1. Evidence of severe cardiovascular, respiratory, urogenital, GI/hepatic, hematologic/immunologic, head, ears, eyes, nose, throat (HEENT), dermatologic/connective tissue, musculoskeletal, metabolic/nutritional, endocrine, neurologic/psychiatric, allergy, major surgery or other relevant diseases as revealed by history, physical examination and laboratory assessments which, in the opinion of the Investigator could interfere with the administration or assessment of study medication. This was to be confirmed by a pretreatment medical examination performed prior to start of the treatment period.

2. Pregnant or lactating females.

3. History of alcohol or drug abuse in the opinion of the Investigator, during the past two years.

4. Subjects who required treatment with non-permitted medication or exceeded the treatment limits of permitted medication (Section 5.4.7.1 and Section 5.4.7.2).

5. Mental disability or any other lack of fitness, in the Investigator's opinion, that precluded subject's participation in or to complete the study.

6. Treatment with any investigational drug within the four weeks prior to entering the study (subject signature and date on the informed consent form).

7. Previous randomization in a cilansetron clinical trial (screen failures from previous cilansetron studies could be considered).

8. History of severe or serious AEs (SAEs) while using any other 5-HT3-receptor antagonists (e.g., alosetron, ondansetron, granisetron, dolasetron), such as severe constipation and/or ischemic colitis.

9. History of drug sensitivity, especially to other 5-HT3 antagonists or food allergy, that in the Investigator's opinion would interfere with either the subject's safety assessments of the study.

10. Subjects with a history of thrombosis/hypercoagulability disorder; subjects with a history of deep vein thrombosis due to trauma or surgery who would otherwise qualify could be included in the study.

11. Known infection with human immunodeficiency virus (HIV).

Specific Gastroenterologic Exclusion Criteria

1. Diarrhea-predominant IBS subjects with a diagnosis of lactose intolerance (as determined by history or a lactose intolerance breath test) whose lactose intolerance symptoms were not completely or substantially relieved solely by abstaining from dairy products. Diarrhea predominant IBS subjects with lactose intolerance on a lactose free diet whom otherwise qualify, may have been enrolled.

2. Presence of organic disease of the GI tract, liver, pancreas, biliary tree (e.g., gastritis, symptomatic gallstones, duodenal ulcer, gastroenteritis, diverticulitis or megacolon) with the exception of hemorrhoids, hiatus hernia, and non-symptomatic gallstones.

3. Suffering from complaints, which were predominantly associated with functional dyspepsia in the opinion of the Investigator.

4. Subjects who rated any upper GI symptoms as severe or intolerable. The Investigator had to ask the subject whether or not they had the following upper GI symptoms over the last four weeks prior to Visit 1: early satiety, postprandial fullness, sensation of prolonged digestion, and nausea. The subject's responses must be included in the subject's source documents.

5. Number of vomiting episodes was greater than one per week.

6. Moderate or severe diverticulosis, in the opinion of the Investigator.

7. Acute diverticulitis or a history of greater than one episode of diverticulitis.

8. History of chronic colitis of any etiology (e.g., ulcerative colitis, Crohn's disease, collagen vascular disease, ischemic colitis). A subject with a history of acute self-limited colitis could be included if otherwise qualified.

9. Acute (currently active) colitis of any etiology.

10. History of intestinal obstruction; stricture, toxic megacolon, GI perforation, fecal impaction.

11. History of laxative abuse as determined by the Investigator.

12. Subjects who responded with moderate, severe or intolerable gastroesophageal reflux disease (GERD) or heartburn symptoms. The Investigator had to ask the subject whether or not they had GERD or heartburn and if so, the severity over the last four weeks prior to Visit 1. The subject's responses must have been included in the subject's source documents.

13. Radiologic or clinical evidence of primary and metastatic GI malignancy, stricture or obstruction of the GI tract, paralytic ileus, or intestinal atony.

14. History of GI bleeding based on clinical judgment that would interfere with the subject's safety assessments of the study, or if the subject had experienced GI bleeding on two or more occasions within six weeks prior to study enrollment (with the exception of blood from hemorrhoids).

15. History of major gastric, hepatic, pancreatic, or intestinal surgery or perforation (excluding cholecystectomy, appendectomy, hemorrhoidectomy or polypectomy).

16. Presence of pathogenic parasites, ova, bacteria or any occult blood in stools which in the opinion of the Investigator could be responsible for GI symptoms (if measured within one month of Visit 1).

17. Abnormal colonoscopy within the last five years (polyps, mild diverticula and hemorrhoids excluded).

18. Any other active or recurring organic disease affecting the GI tract that in the judgment of the principal Investigator would compromise subject safety or interfere with the study assessments.

Drop-Outs

Dropouts were those subjects who left the study earlier than planned for whatever reason. Dropouts were not replaced. Data from dropouts were included in the statistical analysis. The CRF had to be completed up to the time of dropout. All dropouts after the first intake of study medication were to be given a post-study assessment as appropriate. The study termination and final comments in the CRF were to be completed for all dropouts.

Withdrawals

Withdrawals were those dropouts who were discontinued from the study for any of the following reasons:
Adverse events that were intolerable to the subject and/or in the view of the investigator jeopardized the health of the subject;
Any other medical reason;
Insufficient compliance regarding time schedules or medication intake; and
Protocol violation(s) that in the Investigator's opinion was (were) incompatible with further participation of the subject in the study.
   Treatments
   Treatments Administered

The active drug substance used in the 3007, 3008 and 5050 Studies was cilansetron hydrochloride monohydrate, which was supplied as 2 mg tablets. Each tablet contained 2 mg of cilansetron as the hydrochloride monohydrate salt. The dose of cilansetron used in this study was primarily based on results from a recently completed phase II clinical trial (Study S241.2.113) in non-constipated IBS subjects, which showed that the 2 mg dose TID was superior to placebo in providing adequate relief of IBS symptoms. Dosages were manufactured by the Pharmaceutical Supplies Department of Solvay Pharmaceuticals B. V., subject to Good Manufacturing Practice Guidelines. Cilansetron 2 mg was studied for a period of 52 weeks. Study drug was to be taken orally TID with or without food with a glass of water (at least 150 ml).

Prior and Concomitant Therapy

The medications listed in the non-permitted medication section of the double-blind efficacy protocol were not to be used during the treatment period if at all possible. These medications (as with all concomitant medications) had to be documented on the CRF.

Non-Permitted Medication

Subjects requiring chronic or occasional treatment with any of the following drugs were to be excluded from the study. The use of any of these medications during the study was regarded as a protocol violation. Use of these medications had to be documented on the CRF.
Other 5-HT₃ antagonists;
Stimulant laxatives (e.g., bisacodyl, sennosides, cascara, casanthranol);
Osmotic laxatives (e.g., magnesium, phosphate, biphosphate, lactulose);
Anti-diarrheals such as: attapulgite (Diar-Aid®, Kaopectate®, etc.); bismuth subsalicylate (Pepto-Bismol®, Pink Bismuth, and Bismatrol); loperamide (Imodium®, Imodium A-D®, etc.);
5-HT₁-receptor agonist anti-migraine agents such as: sumatriptan (Imitrex®), naratriptan (Amerge®), zolmitriptan (Zomig®), ®), rizatriptan benzoate (Maxalt®);
Pseudoephedrine containing decongestants (e.g., Sudafed®, Entex PSE®, etc.) for more than two days per week during the whole study;
Retinoids such as isotretinoin (Accutane®);
Fluvoxamine Maleate (Luvox® or generic equivalent).
   The GERB recommended that fluvoxamine be a prohibited medication for cilansetron studies based on the results of a pharmacokinetic drug interaction study. It was determined that cilansetron plasma levels increased approximately six to seven times when administered concurrently with fluvoxamine, a selective serotonin reuptake inhibitor. This is believed to be due to fluvoxamine's strong inhibition of the cytochrome P4501A2 pathway. Only small increases in cilansetron plasma concentrations were observed when cilansetron was co-administered with inhibitors of the CYP4502D6 and 3A4 pathways, respectively. Consequently, a substantial increase in cilansetron plasma concentrations is not expected to occur with other selective serotonin reuptake inhibitors.
   Rescue Medication

Non-stimulant laxatives (e.g., glycerin suppositories, enemas, methylcellulose, polyethylene glycol, etc.) could be used to treat constipation. If a subject was having relatively normal bowel movements with respect to frequency and consistency and was feeling occasionally bloated (very mild symptoms), additional use of a stool softener (e.g., docusate), fiber or dietary modifications while keeping the subject on study medication was acceptable in such cases.

Treatment Compliance

Subjects were asked to return unused, partly used or empty bottles to the Investigator at each visit. The Investigator (or designee) was to count the returned tablets and record the number in the CRF and drug accountability log.

If the intake deviated more than 10% of the prefixed number of tablets, the subject was to give an explanation for the discrepancy. The Investigator was to decide whether the subject should continue the study. This was to be documented in the CRF.

Returned medication for each subject was to be counted at each visit by the study monitor, compared with the clinical records of intake, and returned to a contract organization. The Investigator was not permitted to return or destroy unused clinical drug supplies or packaging materials.

Efficacy and Safety Variables

Safety Measurements

The safety of subjects was monitored prior to, during, and at the conclusion of the 3007 and 3008 Studies using the following assessments:
AEs/serious AEs (SAEs)
Laboratory assessments and stool occult blood
Electrocardiograms (ECGs)
Vital signs and body weight
Physical examinations
Concomitant medication use
   The schedule of tests and assessments for all subjects is presented in Table 1.
   Quality of Life Measurements

Subjects were asked to complete the IBS-QOL Survey at Visits 1, 3, and 6 in order to assess possible changes of their QOL. The evaluation of these data followed the common evaluation recommended by the authors of this questionnaire.

TABLE 1
Overview of Assessments - 3007

| | Visit (Day) | | | | | |
|---|---|---|---|---|---|---|
| | 1 | 2 | 3 | 4 | 5 | 6 |
| | (Day 1) | (Day 31) | (Day 91) | (Day 182) | (Day 273) | (Day 365)^{a} |
| Assessments (Periods) | Initial | Interim I | Interim II | Interim III | Interim IV | End of Study |
| Inclusion/Exclusion Criteria | X | | | | | |
| Physical Examination^{b} | X^{c} | | | | | X |
| Vital Signs (Temperature, BP, Pulse) | X^{c} | X | X | X | X | X |
| 12-Lead ECG | X^{c} | X | | X | | X |
| Hematology | X^{c} | X | | X | | X |
| Blood Chemistry | X^{c} | X | | X | | X |
| Urinalysis | X^{c} | X | | X | | X |
| Urine Pregnancy Test^{d} | X | | | | | |
| Quantitative Pregnancy Test^{e} | X^{c} | X | X | X | X | X |
| Blood in Stool | X | X | X | X | X | X |
| Quality of Life Questionnaire | X^{c} | | X | | | X |
| Compliance Check | | | X | X | X | X |
| Distribution of Study Medication | X | | X | X | X | |
| Adverse Events | X^{c} | X | X | X | X | X |
| Concomitant Medication | X | X | X | X | X | X |

BP: blood pressure.
^{a}Or upon early termination.
^{b}Including weight but not height.
^{c}Refers to efficacy protocol assessments at normal end of study visit (termination).
^{d}If positive, the subject was excluded. If negative, a sample for a quantitative serum pregnancy test was drawn and study medications were dispensed.
^{e}Women of child bearing potential only. Subject were instructed to return to the study site for an additional serum pregnancy test if she missed a menstrual period between study visits or otherwise felt that she could be pregnant.

TABLE 2
Overview of Assessments - 3008

| | Pre-Treatment | Treatment Period | | | | | |
|---|---|---|---|---|---|---|---|
| | Visit (time point) | | | | | | |
| | 1 | 2 | 3 | 4 | 5 | 6 | 7 |
| | (Day −14 to −3) | (Day 1) | (Day 31) | (Day 91) | (Day 182) | (Day 273) | (Day 365) |
| | Assessments | | | | | | |
| | Pre-Treatment | Baseline^{h} | Interim I | Interim II | Interim III | Interim IV | End of Studyⁱ |
| Informed consent | X | | | | | | |
| Medical history | X | | | | | | |
| Demographic data | X | | | | | | |
| Physical examination^{a} | X | | | | | | X |
| Vital signs (BP, pulse) | X | | X | X | X | X | X |
| 12-lead ECG | X | | X | | X | | X |
| Hematology^{b} | X^{g} | | X | | X | | X |
| Blood chemistry^{b} | X^{g} | | X | | X | | X |
| Urinalysis^{b} | X^{g} | | X | | X | | X |
| Quantitative pregnancy test^{c} | X | | X | X | X | X | X |
| Parasites ova and stool culture^{d} | X^{g} | | | | | | |
| Blood in stool^{e} | X | X | X | X | X | X | X |
| Colonoscopy^{f} | X | | | | | | |
| Evaluation of diarrhea-predominant | X | | | | | | |
| IBS subjects | | | | | | | |
| Sub-group assessments | X | | | | | | |
| Distribution of Study medication | | X | | X | X | X | |
| Compliance check (study medication) | | | | X | X | X | X |
| Baseline complaints | X | X | | | | | |
| Adverse events | | | X | X | X | X | X |
| Concomitant medication | X | X | X | X | X | X | X |
| Interruption of Activities | X | | | | | | |

^{a}Included height, weight BMI and finger rectal examination at Visit 1; weight at Visit 7.
^{b}Subjects were to be encouraged to fast for at least six hours prior to visit
^{c}Women of child bearing potential only.
^{d}If clinically indicated and had not been done ≦ 1 month before Visit 1.
^{e}Only dispensing of stool cards at Visit 1, only analysis of stool cards at Visit 7, all other visits: analysis of returned stool cards and dispense of new stool cards.
^{f}If not done within five years before Visit 1 or if symptoms had changed since then.
^{g}Repeats or scheduling of procedures had to be performed within two weeks, prior to start of treatment (if necessary).
^{h}If there were more than 14 days between Visit 1 and Visit 2, the Project Medical Director had to be contacted for permission to continue the study.
ⁱOr upon early termination

Pre-Treatment Assessments
Use of Laxatives

At Visit 1 subjects were asked about their laxative use with the following question:

During the last three months, how often have you used laxatives for the relief of constipation ?

0=Zero (never)

1=1 to 3 times (rarely)

2=4 to 6 times (occasionally)

3=7 to 12 times (frequently)

4=more than 12 times (regularly)

Duration of IBS

At Visit 1 subjects were asked about how long they have had IBS with the following question: How long have you been suffering from IBS?

Interruption of Activities

At Visit 1, subjects were asked the following question to determine whether the subjects' bowel problems significantly affected their activities.

Over the past 4 weeks, how have your IBS symptoms significantly interfered with your ability to effectively perform your activities (e.g. work, school, recreation, household, social or travel activities) ?

0=Not at all or rarely

1=Occasionally (more than one-tenth of the time)

2=Often (more than one-quarter of the time)

3=Very often (more than one-half of the time)

4=Almost always

The subject was regarded as a severe IBS subject if he or she answered the above question with a rating of 3 or 4 at Visit 1.

Safety Measurements

Safety assessments in Studies 3007 and 3008 consisted of laboratory testing, AEs, concomitant medications, vital signs, ECG reporting, and physical examination findings. Stool specimens were analyzed at every visit for occult blood. An independent GERB had been established to provide advice on GI AEs during the course of the study.

Constipation

If the subject became constipated during the 3007 and 3008 Studies, the subject was asked the following question:

How does the constipation bother you?

1=Not at all bothered

2=Mildly bothered

3=Moderately bothered

4=Considerably bothered

5=Extremely bothered

This question was asked when the subject reported constipation (either at a visit or spontaneous reporting).

Adverse Events

An AE is any untoward medical occurrence in a subject or clinical investigation subject administered a pharmaceutical product and which does not necessarily have a causal relationship with this treatment. An AE can therefore be any unfavorable and unintended sign, symptom or disease temporally associated with the use of the investigational drug, whether or not related to the investigational drug. It includes any side effect, injury, toxicity or sensitivity, any relevant abnormality (laboratory value, ECG, etc.) and worsening of an existing disease, sign, or symptom.

Adverse events were assessed at Baseline, at each clinic visit, and at the end of the study (Month 12 or the early termination visit). All AEs, which occurred during the study were followed up in accordance with good medical practice until resolved or judged no longer clinically significant, or if a condition was chronic, until fully characterized. Adverse events could occur in the specified follow-up period and, regardless of the interval since the last administration of the study medication, were treated in the same manner as an AE which occurred during the treatment with study medication. Each AE was evaluated for duration, severity, seriousness, and causal relationship to the investigational drug. The action taken and the outcome were also recorded.

Severity:

The severity of the AE was characterized as “mild, moderate, or severe” according to the following definitions:
Mild events are usually transient and do not interfere with the subject's daily activities
Moderate events introduce a low level of inconvenience or concern to the subject and may interfere with daily activities
Severe events interrupt the subject's usual daily activity
   Relationship:

The causal relationship between the study medication and the AE was characterized as unrelated, unlikely, possible, probable, or unknown (unable to judge).
Events are classified as “unrelated” if there is not a reasonable possibility that the study medication caused the AE.
An “unlikely” relationship suggests that only a remote connection exists between the study medication and the reported AE. Other conditions, including chronic illness, progression or expression of the disease state, or reaction to concomitant medication appear to explain the reported AE.
A “possible” relationship suggests that the association of the AE with the study medication is unknown; however, the AE is not reasonably supported by other conditions.
A “probable” relationship suggests that a reasonable temporal sequence of the AE with drug administration exists and, in the Investigator's clinical judgment, it is likely that a causal relationship exists between the drug administration and the AE, and other conditions (chronic illness, progression or expression of the disease state, or concomitant medication reactions) do not appear to explain the AE.
   All efforts were made to classify the AE according to the above categories. The category “unknown” (unable to judge) could be used only if the causality was, for one or another reasons, not assessable, e.g., because of insufficient evidence, conflicting evidence, conflicting data, or poor documentation.
   Serious Adverse Events:

An SAE is any untoward medical occurrence that at any dose:
results in death
is life-threatening (an event in which the subject was at risk of death at the time of the event; it does not refer to an event which hypothetically might have caused death if it was more severe)
results in persistent or significant disability/incapacity
requires (inpatient) hospitalization or prolongation of an existing hospitalization
is a congenital anomaly/birth defect
   In addition, medical and scientific judgment should be exercised in deciding whether other conditions should also be considered serious, such as important medical events that may not be immediately life-threatening or not result in death or hospitalization, but may jeopardize the subject's safety or may require intervention to prevent one of the other outcomes listed in the definition above. If a subject became pregnant during drug administration, this was to be reported as a SAE.
   Laboratory Assessments

Subjects were encouraged to fast at least six hours prior to laboratory samples being drawn. Laboratory safety tests were performed at Day 1 (Visit 5 of the core protocol), Months 1, 6, and 12 (end of study or at the early termination visit). Blood and urine were analyzed for:

Hematology:

Prothrombin time (PT) (only before colonoscopy, as per clinical practice)
Hemoglobin
Hematocrit
Red blood cell count (RBC)
White blood cell count (WBC)
Differential blood count
Platelet count
   Blood Chemistry:
Glucose
Creatinine
Blood urea nitrogen (BUN)
Creatine kinase (CK)
Alkaline phosphatase (AP)
Total bilirubin
Alanine aminotransferase (ALT/SGPT)
Aspartate aminotransferase (AST/SGOT)
Gamma-glutamyltransferase (GGT)
Triglycerides
Cholesterol
Total protein
Uric acid
Sodium
Potassium
Calcium
Quantitative beta human chorionic gonadotrophin (β-HCG) (at every visit)
Thyroid stimulating hormone (visit 1 only)
   Urine:
Urobilinogen
Blood
Bilirubin
Ketones
Glucose
Protein
Nitrite
Leukocytes
pH
Dipstick pregnancy test (Day 1 only, if necessary)
   Stool:
Parasite, ova and stool culture (visit 1 only, if necessary)
Occult blood (analysis at each visit)

Quantitative β-HCG tests were performed at each visit, to determine whether female subjects of childbearing potential were pregnant. Additionally, a dipstick urine pregnancy test was performed at Visit 1. If the urine test was positive, the subject was excluded. If the urine test was negative, a sample was drawn for the quantitative β-HCG test and the subject was dispensed study medication.

If a subject was pregnant based on serum pregnancy test the subject was removed from study medication and all early termination procedures (including reporting of SAEs) were completed. Furthermore, the subject was to be followed until resolution of the pregnancy. If the subject's β-HCG pregnancy test was indeterminate, the subject had to discontinue study medication for a period not to exceed two weeks and have a repeat pregnancy test. If the repeat test was negative the subject could continue study medication. A female subject of childbearing potential was instructed to return to the study site for a quantitative pregnancy test if she missed her normal monthly menstrual period between scheduled study visits or otherwise felt that she could be pregnant.

For stool analysis for occult blood, subjects were given three stool cards at Day 1, Months 1, 3, 6, and 9 with instructions to place one stool sample from a single day's stool on each card and bring the three cards back at their next visit. The three stool samples had to come from stools collected on separate days during the last week prior to their next scheduled visit (i.e., one stool sample per card). At each visit the study staff reviewed the three stool sample cards and recorded the results in the CRF.

At Day 1 (Visit 5 of the core protocol), laboratory samples were drawn and the results were not received until after the subject had been dispensed study medication and enrolled into this extension protocol. Therefore, upon receipt of the laboratory results the investigator (or designee) determined if the results interfered with the continued administration of study medication. At this time, the investigator had to determine if the subject was suitable for continuation into the study (see Section 0 Exclusion Criteria).

Vital Signs and Body Weight

Vital signs (including systolic and diastolic blood pressure, temperature, and pulse) were assessed at all clinic visits. Weight was obtained at Day 1 (Visit 5 of the core protocol) and Month 12 (end of study or early termination visit).

Electrocardiograms

Resting 12-lead ECGs were performed at Day 1 (Visit 5 of the core protocol), Months 1, 6, and 12 (end of study or early termination visit). A centralized ECG service was used to evaluate all ECG tracings to supply consistent standardized ECG interpretation.

Physical Examinations

Complete physical examinations were performed at Day 1 (Visit 5 of the core protocol) and Month 12 (end of study or early termination visit).

Concomitant Medications

Prior and concomitant medications were recorded during each visit and/or through spontaneous reporting. Administration of all medications (including over the counter, herbal/holistic, and prescription medications) was documented in the CRF.

Quality of Life Measurement

Subjects were asked to complete a IBS-QOL Survey at Day 1 (Visit 5 of the core protocol), Month 6 and Month 12 (end of study or early termination) in order to assess possible changes of their QOL. The IBS-QoL survey, a 34 item IBS-specific QoL questionnaire consisting of 8 subscales (i.e., interference with activity; body image; health worry; food avoidance; social reaction; sexual; relationship; and dysphoria) was completed by patients at baseline and at later specified time points (e.g., at week 12 and at week 16 in Study 5050). The evaluation of these data followed the common evaluation recommended by the authors of this questionnaire.

Appropriateness of Measurements

All safety assessments used in this study are widely used and generally recognized as reliable, accurate, and relevant. The definition of IBS is based on the Rome criteria published in 1988, and revised in 1990 and 1992. The definition of diarrhea-predominant IBS is based on the Rome II criteria. Rome criteria are the standard used to define IBS for the purpose of clinical studies for functional bowel diseases.

Data Quality Assurance

Steps were taken to assure the accuracy and reliability of data. These included: the selection of qualified Investigators and appropriate study centers; the review of protocol procedures with the Investigator and associated personnel prior to the study; periodic monitoring visits; and on-site audits. The CRFs were reviewed for accuracy and completeness, 100% verified for critical fields, and the database was audited.

Statistical and Analytical Plan

Summary statistics for the data collected during the studies are presented to give a general description of the subjects studied and an overview of the safety results. Data from all centers were combined in the computation of these descriptive summaries. Categorical variables were summarized by the number and percentage of subjects in each category. Continuous variables were summarized using N, mean, standard error, median, minimum, and maximum values. No formal statistical tests were planned a priori, but if necessary, statistical tests were to be used to explore trends over time and relationship with cilansetron use. Important results are presented as in-text tables.

Analysis Populations

The Safety populations in Studies 3007 and 3008 were defined as all subjects who received at least one dose of study medication in Studies 3007 and 3008, respectively, and had at least one assessment of safety after starting study medication in Studies 3007 and 3008, even if no safety evaluations were carried out and no AEs were reported. Specifically, subjects who were lost to follow-up and had no post-Baseline contact with the site were excluded from the Safety population. The subjects who discontinued from the study due to lost to follow-up were identified based on the information collected on the study termination CRF page.

Analysis Timepoints—3007

Analyses of Study 3007 were based on data from the 3006 Study (discussed above) as well as data from Study 3007. Except where noted, analyses were based on data collected while subjects were receiving cilansetron treatment in either Study 3006 or Study 3007. Specifically, analyses were based on on-treatment data from both Study 3006 and 3007 for subjects randomized to cilansetron in Study 3006, and were based on on-treatment data from Study 3007 for subjects randomized to placebo in Study 3006.

All by-visit analyses were relative to the start of cilansetron study medication. Therefore, a correspondence had to be made between visit timepoints in Study 3006 and Study 3007 for subjects randomized to cilansetron in Study 3006 and visit timepoints in Study 3007 for subjects randomized to placebo in Study 3006. Table 4 summarizes the correspondence between the analysis timepoints and corresponding visit numbers of Studies 3006 and Study 3007, along with the target days and visit windows.

TABLE 3
Analysis Timepoints

| Analysis | Visit Number | Visit Number |
|---|---|---|
| Timepoint | (Visit Window; | (Visit Window; |
| (Relative | Target Day) for | Target Day) for |
| to the Starting | 3006 subjects | 3006 subjects |
| of Cilansetron) | randomized to cilansetron | randomized to placebo |
| Day 1 | 3006 Visit 2 (Day 1) | 3007 Visit 1 (Day 1) |
| Month 1 | 3006 Visit 3 | 3007 Visit 2 |
| | (Day 16 to 46; Day 31) | (Day 16 to 46; Day 31) |
| Month 3 | 3006 Visit 5 | 3007 Visit 3 |
| | (Day 76 to 106; Day 91) | (Day 76 to 106; Day 91) |
| Month 6 | 3007 Visit 3 | 3007 Visit 4 |
| | (Day 137 to 227; Day 182) | (Day 137 to 227; Day 182) |
| Month 9 | 3007 Visit 4 | 3007 Visit 5 |
| | (Day 228 to 318; Day 273) | (Day 228 to 318; Day 273) |
| Month 12 | 3007 Visit 5 | 3007 Visit 6 |
| | (Day 319 to 410; Day 365) | (Day 319 to 410; Day 365) |
| Month 15 | 3007 Visit 6 | N/A |
| | (≧Day 411; Day 455) | |

For all analyses, Month 1 refers to Month 1 from Study 3006 for subjects randomized to cilansetron in Study 3006 and Month 1 from Study 3007 for subjects randomized to placebo in Study 3006. Since there was no clinic visit at Month 2 for Study 3007, Month 2 visit of the core Study 3006 was not included in any visit interval. For subjects who were on cilansetron in core Study 3006, the pre-treatment period of Study 3006 was the analysis Baseline.

For vital signs, laboratory results, and ECGs, Baseline was defined as the pre-treatment period of core Study 3006 for subjects randomized to the cilansetron treatment group in Study 3006 and as Visit 5 of Study 3006 for subjects randomized to the placebo group in Study 3006. Data reported at Visit 1 of Study 3006 were used as a Baseline for following parameters: medical history, assessment of Interruption of Activities, and demographics.

For IBS-QOL, Baseline for subjects randomized to the cilansetron treatment group was defined as Visit 2 (Day 1) of core Study 3006. For subjects randomized to the placebo group of core Study 3006, Visit 5 of the core Study 3006 was assigned as the Baseline.

If there were multiple assessments within a visit window with non-missing values, the assessment with the closest date to the target date was used in the analysis. If there were multiple assessments with the same closest date to the target date, the assessment with the latest visit date was used. If the multiple assessments, such as laboratory parameters or ECGs, with the same closest date to the target date were performed on the same visit date, the last assessment was used. Data collected outside the visit windows were not included in the by visit analysis, but could be included in overall and endpoint analyses.

All visit windows were calculated inclusive of start and stop days for the window. The above visit windows could result in observations not being included in the by-visit analyses because the purpose of the visit windows was to allow for more homogenous visit data for the by-visit analyses. However, all observations collected up through 30 days after the last dose of study drug were included in any markedly abnormal presentations or clinically significant data presentations.

Similar to the safety assessment mentioned above, the first day of receiving cilansetron was used as the reference timepoint for the accounting of time interval analyses. Thus starting point here was assigned to Day 1 of Study 3007 for subjects from the placebo group of Study 3006, compared to Day 1 of the core Study 3006 for those from the cilansetron group of Study 3006. For the by time interval analyses of disposition and prevalence of AEs, the following interval windows were used:
Interval 1: 0 to 1 month: [Day 1 through Day 30; does not apply to summary of prevalence of AEs]
Interval 2: 1 to 3 months: [Day 31 through Day 91; does not apply to summary of prevalence of AEs]
Interval 4: 0 to 3 months: [Day 1 through Day 91]
Interval 5: 3 to 6 months: [Day 92 through Day 182]
Interval 6: 6 to 9 months: [Day 183 through Day 273]
Interval 7: 9 to 12 months: [Day 274 through Day 365]
Interval 8: 12 to 15 months: [Day 366 through Day 456]
   Interval 9: >15 months: [≧Day 457]
   Analysis Time Points—3008

Study 3008 was comprised of a pre-treatment period and a treatment-period. After the pretreatment assessment at Visit 1 (Days 0.14 to −3), subjects were to return to the study site for a Baseline assessment and start the treatment at Visit 2 (Day 1). Interim assessments were done at Visits 3 (Day 31/Month 1), 4 (Day 91/Month 3), 5 (Day 182/Month 6), and 6 (Day 273/Month 9). Subjects completed the study with an end of study assessment at Visit 7 (Day 365/Month 12). All subjects who discontinued study treatment (early termination) after Visit 2 (Day 1) had to have the same post study assessments required at Visit 7 performed at their early termination visit.

For by-visit assessments, visit windows were assigned to the post-Baseline assessments as follows (vital signs, laboratory tests, and ECGs):

Month 1: Day 16 to Day 46; the target Day is day 31;

Month 3: Day 76 to Day 106; the target Day is day 91;

Month 6: Day 137 to Day 227; the target Day was day 182;

Month 9: Day 228 to Day 318; the target Day is day 273;

Month 12: Day 319 to Day 410; the target Day is day 365.

If there were multiple assessments within a visit window with non-missing values, the assessment with the closest date to the target date was used in the analysis. If there were multiple assessments with the same closest date to the target date, the assessment with the latest visit date was used. If the multiple assessments, such as laboratory parameters or ECGs, with the same closest date to the target date were performed on the same visit date, the last assessment was used. Data collected outside the visit windows were not included in the by-visit analysis, but may have been included in overall and Endpoint analyses. All visit windows were calculated inclusive of start and stop days for the window. The above visit windows could result in observations not being included in the by-visit analyses because the purpose of the visit windows was to allow for more homogenous visit data for the by-visit analyses. However, all observations collected up through 30 days after the last dose of study drug were included in any markedly abnormal presentations or clinically significant data presentations.

For the by time-interval analyses of disposition, prevalence of AEs, and of markedly abnormal and predefined changes in laboratory values, vital signs, and ECGs, the following interval windows were used:

Interval 1: 0 to 1 month: [Day 1 through Day 30; does not apply to summary of prevalence of AEs]

Interval 2: 1 to 2 months: [Day 31 through Day 60; does not apply to summary of prevalence of AEs]

Interval 3: 2 to 3 months: [Day 61 through Day 91; does not apply to summary of prevalence of AEs]

Interval 4: 0 to 3 months: [Day 1 through Day 91]

Interval 5: 3 to 6 months: [Day 92 through Day 182]

Interval 6: 6 to 9 months: [Day 183 through Day 273]

Interval 7: >9 months: [≧Day 274]

Data Handling Rules

The following data handling rules were established to ensure the quality of data used in the analyses:
All data from core Study 3006 were derived from the individual study source and analysis datasets, including coded files such as AEs, medical history, and concomitant medication. No additional measures were taken to resolve any coding consistencies between the core Study 3006 and Study 3007. Such discrepancies were expected to occur with very limited frequency.
Data provided by vendors for standard laboratory parameters and ECGs were reconciled at the subject level with the CRF database to identify and correct discrepancies in subject identification, and demographic information.
If the onset date of an AE in the 3007 or 3008 CRF database was missing, then the event on the AE CRF was considered as treatment-emergent, unless the identical even was recorded on the baseline complaint CRF, then the event was not considered as treatment-emergent.
Baseline for laboratory values, vital signs, physical examination, and ECGs was defined as the last measurement prior to or on the date of the first dose of study medication, e.g., as Visit 1 of core Study 3006 for Study 3007 for subjects randomized to the cilansetron treatment group in Study 3006 and as the last post-Baseline visit of Study 3006 (normally Visit 5) for subjects randomized to the placebo group in Study 3006. These Baseline values from Study 3006 were used for summaries of change from Baseline.
For by-timepoint or by-visit analysis, Endpoint for laboratory values, vital signs, physical examination, and ECGs was defined as the last non-missing measurement collected while on cilansetron study medication. On cilansetron study medication was defined as any value collected within one day of the last dose of cilansetron.
If no assessments were done in Study 3007 for laboratory values, vital signs, and ECGs for a subject from the treatment group of Study 3006, then endpoint for that subject could be carried over from Study 3006 data.
For the presentation of abnormalities for laboratory measurement, vital signs, and ECGs, abnormal values collected after the first dose of study medication and within 30 days of the last dose of study medication were included.
   Subject Disposition

In Study 3007, this was comprised of the number and percentage of subjects who continued into Study 3007 from Study 3006, subjects who received study medication, subjects who completed the study, and subjects who prematurely discontinued from the study are presented. In Study 3008, this comprised the number and percentage of subjects who enrolled in the study (i.e., signed informed consent), subjects who received study medications, subjects who completed the study, and subjects who prematurely discontinued from the study. The reasons of discontinuation from the study were summarized. Numbers were broken down further by reasons of discontinuation.

A listing of subjects in the Safety population who prematurely discontinued from the study with reasons for discontinuation is presented in Section 12. Also, a listing of subjects who had a major protocol deviation will be presented for the Safety population in Section 12.

The number of subjects not satisfying the inclusion criteria or satisfying the exclusion criteria (general exclusion criteria, specific GI exclusion criteria, and additional interim exclusion criteria) is presented for all subjects in the safety population.

The number of the subjects who had major protocol deviations during the study is presented for the safety population. Criteria for major protocol deviations were defined as:
Subjects who did not meet inclusion/exclusion criteria other than the inclusion criteria 6 and 7, even if a waiver was granted; subjects who did not meet inclusion criterion 6 or 7 and met all other inclusion/exclusion criteria will not be considered major protocol violators;
Subjects who were <80% or >120% compliant with study drug; or
Subjects who received any of the following medications, prohibited by the protocol, after starting the treatment period:
stimulant laxatives
osmotic laxatives
other 5HT3 antagonists
anti-diarrheals
5HT1 agonists
Pseudoephedrine containing decongestants

For Study 5050, of a total of 402 patients randomized to treatment, 400 received study medication and 395 were included in the safety analysis (cilansetron, N=301; placebo, N=94). Five patients were excluded because of a lack of a post-baseline safety evaluation. Thirty nine percent of patients in the cilansetron group and 56% in the placebo group withdrew from the study; the differences in withdrawals largely were due to lack of efficacy (15% v. 34%, respectively), followed by other unspecified reasons (16% v. 19%, respectively) and AEs (10% v. 9%, respectively). Most patients (57%) were in the 41 to 64 year age category and 72% of all patients were female.

Demographic Variables, Background Characteristics, and Medical History

The following Baseline demographic variables are presented for the Safety population: age (years); age category (18-40, 41-64, and ≧65 years old); gender; race (Caucasian, African American, Asian, other); and current use of tobacco products (yes, no). The age categories may be collapsed or expanded further depending on whether it mitigates the interpretation. Age will be calculated at the date of the first dose of study medication, using date of birth information. The number and percentage of subjects with laxative use for the relief of constipation during the last three months prior to enrollment are presented by category of use [never, rarely (1 to 3 times), occasionally (4 to 6 times), frequently (7 to 12 times), and regularly (more than 12 times)]. Prior laxative use is categorized as No (response of never) and Yes [responses of 1 (1 to 3 times), 2 (4 to 6 times), 3 (7 to 12 times), and 4 (more than 12 times)].

Summary statistics are presented for the Safety population for the following Baseline characteristics and vital signs: weight, height, body mass index (BMI), pulse rate, systolic blood pressure, diastolic blood pressure, and body temperature. The number and percentage of subjects in each BMI category are also presented (<30 and ≧30 kg/m²). For vital signs of Study 3007, Baseline was defined as Visit 1 of core Study 3006 for subjects randomized to the cilansetron treatment group in Study 3006, and as the last post-Baseline visit of Study 3006 (normally Visit 5) for subjects randomized to the placebo group in Study 3006. For height, weight, and BMI, Visit 1 of core Study 3006 was assigned as Baseline for all subjects of Study 3007.

The number and percentage of subjects in the safety population in each assessment category at the pre-treatment visit of Study 3008 are presented for the following organic or parasite diseases: parasites (negative, positive, not applicable (NA)); ova (positive, negative, NA); stool culture (normal; abnormal; NA); and colonoscopy (normal; abnormal).

The Interruption of Activities assessment performed at the pre-treatment visit of Studies 3007 and 3008 (e.g., at the beginning of Study 3006) was summarized for subjects in the Safety population. The number and percentage of subjects with each assessment response at the pre-treatment visit were summarized [not at all or rarely, occasionally (more than one-tenth of the time), often (more than one-quarter of the time), very often (more than one-half of the time), almost always]. The severity of IBS was categorized based on the response to the Interruption of Activities Assessment at the pre-treatment visit. Severe IBS included a response of 3 (very often) or 4 (almost always), moderate IBS included a response of 1 (occasionally) or 2 (often), and mild IBS will include a response of 0 (rarely or not at all).

Baseline IBS History/Disease definition was summarized for the Safety population. The number and percentage of subjects with continuous or recurrent symptoms for at least three months of abdominal pain and discomfort which is: relieved with defecation, associated with a change in stool frequency, or associated with a change in stool consistency are presented. The frequency of the following characteristics was also summarized (none, occasional, frequent, or permanent): altered stool frequency (<3 stools/week, >3 stools/day); altered stool form (lumpy/hard, loose/watery), and altered stool passage (straining, urgency, feeling of incomplete evacuation, passage of mucus, bloating, and feeling of abdominal distension). Summary statistics are also provided for the duration of IBS. Duration of IBS is categorized into the following categories: <6 months, 6 month to <12 months, 12 months to <18 months, 18 months to <24 months, and ≧24 months. The duration categories may be collapsed or expanded further depending on whether it mitigates the interpretation.

The number and percentage of subjects in each response category (not at all or rarely, occasionally (more than one-tenth of the time), often (more than one-quarter of the time), very often (more than one-half of the time), and almost always for the following items collected during the studies on the diarrhea-predominant IBS subject eligibility questionnaire are presented for the Safety population:
In the last six months, have you had more than three bowel movements per day?
In the last six months, have you had loose, mushy, or watery bowel movements (stools)?
In the last six months, have you experienced an urgent need to have a bowel movement that made you rush to a toilet?
In the last six months, have you had fewer than three bowel movements each week?
In the last six months, have you had lumpy or hard bowel movements (stools)?
In the last six months, have you needed to strain a lot to have a bowel movement?

Medical history diagnoses/conditions were coded using the Medical Dictionary for Regulatory Activities (MedDRA) coding dictionary. The incidence of medical history conditions by primary system organ class and preferred term is presented for the Safety population. Subjects were counted at most once within each primary system organ class and preferred term.

A listing of the Baseline demographic data, including age, gender, race, use of tobacco, BMI, duration of IBS, and severity of IBS is presented for all subjects in the Safety population.

Prior and Concomitant Medications

Concomitant medications were assigned to a generic drug name using the World Health Organization (WHO) Drug dictionary.¹ Subsequently, the drug names were matched to the respective Anatomical-Therapeutic-Chemical (ATC) classification system.

Concomitant Medications Taken after Receiving Study Medication

Concomitant medications were defined as any medication started on or after the first dose of cilansetron study medication, or any medication started prior to first dose of cilansetron study medication and continued after the first dose of cilansetron.

For Study 3007, if a medication record from the Study 3006 database would qualify as concomitant medication in 3007 for a subject randomized to the placebo treatment group in Study 3006, based on conservative rules applied to handle partial dates, then the medication was classified as concomitant medication for the Study 3007 analysis.

The incidence of key concomitant medications was summarized by ATC class (the 3^{rd} level if available; the 2^{nd} level otherwise) and generic term for the Safety population. The following medications were considered key concomitant medications:
Anti-diarrheals
Analgesics
Anti-migraine isometheptene mucate/dichloralphenazone/acetaminophen combinations
Pseudoephedrine containing decongestants
Low dose oral steroids
Prophylactic antibiotics
Anti-depressants
Fiber supplements
Psyllium hydrophilic mucilloid
Non-narcotic analgesics for the treatment of chronic pain associated with chronic arthritis, fibromyalgia, or sciatica
Calcium antagonists
Non-stimulant laxatives
Stimulant/irritant laxatives
Anticholinergics
Antispasmodics
Anti-emetics
Oral blood glucose lowering agents
Serum lipid reducing agents
Anti-anemic preparations
Anti-acne preparations for systemic use
Cough and cold preparations

A list of ATC codes corresponding to each medication class listed above is provided in Table 4.

TABLE 4
Key Concomitant Medication ATC Code List

| Key Concomitant Medication | Supporting ATC Codes | Comments/Notes |
|---|---|---|
| Anti-diarrheals | A03; A07DA; A07BB; | Antispasmodic/anticholinergic/propulsive agents; |
| | A07BC; A07FA; A07X | antipropulsives; bismuth; other intestinal |
| | | absorbents; antidiarrheal microorganisms; other |
| | | anti-diarrheals |
| Analgesics | N02A; N02B | Opioids; other analgesics & antipyretics |
| Anti-migraines | N02CA; N02CB; N02CX | |
| Pseudoephedrine decongestants | R01AA; R01AB; R01BA | |
| Low dose oral steroids | A01AC; H02 | Corticosteroids for local oral treatment; |
| | | corticosteroids for systemic use |
| Prophylactic antibiotics | A01AB; D10AF; D01BA; | Anti-infectives for local oral treatment; acne anti- |
| | G01A; G01BA, GO1BC, | infectives; dermatologic anti-fungals; gyn anti- |
| | G01BE, G01BF; G04AA, | infectives/anticeptics; gyn antibiotics/steroid |
| | G04AB, G04AC, G04AG, | combos; urinary anti-infectives; systemic |
| | G04AH, G04AK; J01; J02; | antibacterials; systemic anti-mycotics; systemic |
| | J04; J05 | antimycobacterials; systemic antivirals; |
| Anti-depressants | N06A | Entire class |
| Fiber supplements | A06AC | Bulk producers |
| Psyllium hydrophilic mucilloid | A06AC | Bulk producers |
| Non-narcotic analgesics | N02B | All but opiates |
| Calcium antagonists | C08; C02LI | Ca-channel antagonists; Ca-channel antagonist & |
| | | diuretic combinations |
| Non-stimulant laxatives | A06AA; A06AC; A06AD; | Softeners & emollients; bulk producers; |
| | A06AG; A02AA | osmotically acting; enemas; magnesium |
| | | compounds; |
| Stimulant/irritant laxatives | A06AB | Contact laxatives |
| Anticholinergics | A03; R06AA; R06AD; | R06 = Systemic antihistamines, specifically: |
| | N05AA, N05AB, N05AC | R06AA-ethanolamines (i.e., diphenhydramine & |
| | | clemastine) have high anticholinergic activity as |
| | | do R06AD-phenythiazines (i.e., promethazine); |
| | | phenylthiazine containing antipsychotics (N05A- |
| | | A, B & C) |
| Antispasmodics | A03A A03B A03C A03D | Antispasmodic & anticholinergic and propulsive |
| | A03E A03FA; | agents; antacids with antispasmodics; urinary |
| | A02AG; G04BD | antispasmodics |
| Anti-emetics | A04AA & A04AD; | R06AE-piperazine antihistamines for systemic use |
| | R06AE; R06AD; | (i.e., hydroxyzine) have high anti-emetic activity |
| | | & R06AD-phenylthizaines have very high anti- |
| | | emetic activity |
| Oral blood glucose lowering | A10BF | Alpha glucosidase inhibitors |
| agents | | |
| Serum lipid reducing agents | B04AD | Bile acid sequestrants |
| Anti-anemic preparations | B03A | Iron preparations |
| Anti-acne preparations for | D10BA; D05BB | Retinoids for acne; retinoids for psoriasis |
| systemic use | | |
| Cough & cold preparations | R05DA; R05FA | Opium alkaloids & derivatives; opium derivatives |
| | | & expectorants |

Compliance and Duration of Exposure

Percentage compliance was calculated as the number of tablets a subject took divided by the number of tablets scheduled to be taken multiplied by 100. For the Safety population, the number and percentage of subjects with percentage compliance in each of the following categories were summarized: <80%, 80%-120%, and >120%. Summaries showing the number of subjects, mean and median compliance, standard error, minimum, and maximum compliance are also presented. A listing of compliance to the study medication is presented in Section 12 of this report.

Duration of exposure to study medication was calculated from the day that the first dose of cilansetron study medication was taken, to the last day cilansetron was taken (last date study medication was taken minus first date cilansetron study medication was taken plus one), without adjustment for drug holidays. The mean duration of exposure was summarized in days for the Safety population for the entire duration of the study. The duration of exposure was also categorized and presented as 0-1 month, >1-2 months, >2-3 months, >3-6 months, >6-9 months, >9-12 months, >12-15 months, and >15 months.

Drug holiday information collected during Studies 3007 and 3008 was summarized for the Safety population for the period when subjects were on cilansetron study medication. In Study 3007, this period included treatment time for subjects from the placebo group of Study 3006. For those from the treatment group of Study 3006, however, treatment time in both Study 3006 and 3007 were included. A drug holiday was when the investigator directs a subject to discontinue study medication for a period of time (not to exceed seven days without approval from Project Medical Officer) due to the subject well being and when the subject was on cilansetron. The number and percentage of subjects with at least one occurrence of drug holiday (overall and by time interval), the distribution of number of drug holidays during the entire treatment period, the distribution of total number of days on drug holiday during the entire treatment period, descriptive statistics of total number of days on drug holiday during the entire treatment period, and the ratio of duration of drug holiday over the duration of treatment are presented.

Overall Adverse Event Analysis

In the calculation of the percentages for AEs that were gender specific, the denominator was based on the number of subjects in the Safety population for that gender. Gender specific AEs were flagged in the AE tables.

A treatment-emergent AE (TEAE) was defined as any AE with onset date on or after the date of starting study medication (i.e., for Study 3007, the date of first dose of study medication from Study 3006 for patients randomized to the cilansetron treatment group in Study 3006, or the date of first dose from Study 3007 for patients randomized to the placebo treatment group in Study 3007) and up through seven days after the last dose of study medication (if not serious) or up through 30 days after the last dose of study medication (if serious). Adverse events that were already present prior to the first dose of cilansetron study medication but increased in severity afterwards were considered as treatment-emergent.

For Study 3007, if a subject who randomized to the cilansetron treatment group in Study 3006 temporarily discontinued study medication after the last dose of study medication from 3006, then any AE with onset between the last dose of study medication for Study 3006 and the first dose of study medication for Study S241.3007 were considered treatment-emergent. Moreover, if an AE from the 3006 database would qualify as TEAE in Study 3007 for a subject randomized to the placebo treatment group in Study 3006 based on conservative rules applied to handle partial dates, then the AE was not classified as treatment-emergent for analyses of Study 3007.

The overall incidence of TEAEs is presented by primary system organ class and by preferred term. Subjects were counted at most once for each preferred term and each primary system organ class. The overall incidence of TEAEs is also presented by primary system organ class and higher level term, as well as by primary system organ class and lower level term. In addition, the overall incidence of TEAEs is presented by all system organ classes and all higher level terms.

The overall incidence of TEAEs is also presented separately by severity and by drug relationship. For these presentations, subjects were counted at most once for each preferred term and each primary system organ class and each preferred term under the maximum severity or the strongest relationship to study medication. Severity was categorized as mild, moderate, severe, or unknown; for drug relationship, the pooling of categories was represented as not related (unrelated), related (possibly related, probably related, and unlikely), and unknown.

The prevalence of all TEAEs is presented by three-month interval defined in Section 0 (i.e., Months 0-3, 3-6, 6-9, 9-12, and 12-15). In the analyses of prevalence, TEAEs were counted during each interval in which they occurred, regardless of whether they began during that interval. Subjects were counted at most once for each preferred term and each primary system organ class for each time interval. Subjects were counted in the denominator of these calculations only if they were followed during the time interval of interest. All TEAEs reported through a subject's study discontinuation visit and all SAEs reported within 30 days of permanent discontinuation of study medication were included. In the calculation of the prevalence, percentages for TEAEs that were gender specific were based on the number of subjects in the Safety population for that gender.

A listing of all AEs (TEAEs as well as non-TEAEs) is presented. This listing shows the preferred term of the AE, severity, seriousness, action taken, outcome, and relationship of the AE, start and stop date of the AE, study day of onset of the AE, age, race, and gender; non-TEAEs are identified.

The overall incidence of TEAE of special interest was summarized by primary system organ class and preferred term. Events of special interest were selected prior to database lock and were not based on knowledge of occurrence of these events in this study. Kaplan-Meier curves for the time to onset of selected AEs of special interest (e.g., constipation and ischemic colitis) are presented. The selected AEs were identified prior to database lock.

Additional analyses were performed for the special interest AE of constipation. The number and percentage of subjects (overall and by gender) with at least one episode of constipation, subjects with at least one episode of serious constipation, subjects with at least one episode of severe constipation, subjects with at least one episode of constipation that was related to the study medication, subjects with constipation that led to discontinuation from the study, subjects with constipation that led to dose reduction, and number of subjects with serious complication of constipation (i.e., ischemic colitis, fecal impaction, obstruction, perforation, mega rectum, and colectomy) were presented. The number and percentage within each category of number of constipation episodes (0, 1, 2, 3, 4, and ≧5) and within each bother score (not at all bothered, mildly bothered, moderately bothered, considerably bothered, and extremely bothered) were presented. A subject was counted once with the maximum bother score of constipation he/she experienced during the treatment period. Summary statistics for the duration of constipation episodes were presented (duration of constipation was defined using the onset and resolution dates in the AE CRF). A listing of all subjects with constipation and their drug holiday information (e.g., number of drug holidays and duration of drug holidays) is presented.

The incidence of TEAEs occurring in ≧5% of the subjects in the Safety population was summarized using primary system organ class, higher level term, and preferred term. The 5% cutoff point was based on the incidence within higher level terms. The presentation was sorted by descending frequency of the incidence.

Adverse Events Subgroup Analyses—Drug-Gender and Drug-Age Interactions

Summaries of TEAEs occurring in ≧5% of the subjects in the Safety population were presented by gender, age category (18-40, 41-64, and ≧65 years), and overall. The 5% cutoff point was based on the incidence within higher level terms.

Serious Adverse Events

The overall incidence of treatment-emergent SAEs was summarized by primary system organ class and preferred term, if there were a sufficient number of SAEs to warrant such a presentation. Subjects were counted at most once for each primary system organ class and preferred term. Serious AEs that resolved prior to receiving study medication or occurred more than 30 days after the last dose of study medication were not included in the incidence tables.

A listing of all SAEs is presented; SAEs with onset prior to receiving study medication or more than 30 days after discontinuation of study medication were flagged. The listing presents lower level term and preferred term of the SAE, severity, action taken, outcome, and relationship of the SAE, start and stop date of the SAE, study day of onset of the SAE, age, race, and sex.

Narratives were prepared for all subjects with a SAE, including deaths.

Because the anticipated database lock for Study 3007 was within 30 days after the last subject visit, there was the potential that an SAE could occur within 30 days after the last subject dose, but not be included in the scientific database. If any such event occurred, the event is identified in a footnote of the SAE tables, and is discussed as special cases in the study report. The database was not unlocked to include any such additional events.

For Study 3008, summaries of TEAEs occurring in ≧5% of the subjects in the safety population were presented by gender, age (18-40, 41-64, and ≧65 years), and country. The 5% cutoff point was based on the incidence within higher level terms.

Serious Adverse Events

The overall incidence of treatment-emergent SAEs was summarized by primary system organ class and preferred term, if there were a sufficient number of SAEs to warrant such a presentation. Subjects were counted at most once for each primary system organ class and preferred term. Serious AEs that resolved prior to receiving study medication or occurred more than 30 days after the last dose of study medication were not included in the incidence tables. A listing of all SAEs is presented; SAEs with onset prior to receiving study medication or more than 30 days after discontinuation of study medication were flagged. The listing presents lower level term and preferred term of the SAE, severity, action taken, outcome, and relationship of the SAE, start and stop date of the SAE, study day of onset of the SAE, age, race, and sex.

Adverse Events that Led to Dose Reduction of Study Medication

The total number of subjects with at least one TEAE leading to dose reduction is presented, if there were a sufficient number of AEs leading to dose reduction to warrant such a presentation. The overall incidence of TEAEs leading to dose reduction of study medication was summarized for all subjects by preferred term and by primary system organ class. Subjects were counted at most once for each primary system organ class and preferred term. Adverse events leading to dose reduction were identified as events with an action taken of “reduce dose”.

Adverse Events that Led to Permanent Discontinuation of Study Medication

The overall incidence of TEAEs leading to permanent discontinuation of study medication is presented by primary system organ class and preferred term. Subjects were counted at most once for each primary system organ class and preferred term.

Laboratory Parameters

Laboratory data are presented in conventional units. Only one central laboratory was used for each laboratory parameter, thus no laboratory normalization was carried out.

For Study 3007, “baseline” was defined as Visit 1 of core Study 3006 for subjects randomized to the cilansetron treatment group in Study 3006 and as the last post-Baseline visit of Study 3006 (normally Visit 5) for subjects randomized to the placebo group in Study 3006.

Summary statistics are presented for all hematology and chemistry parameters at each analysis timepoint and at Endpoint, as well as for change from Baseline results at each post-Baseline analysis timepoint and at Endpoint. For laboratory assessments that were gender specific, summaries were presented separately for males and females. A listing of laboratory assessments for hematology, chemistry, and urinalysis parameters is presented. The following categories were used for the urinalysis parameter summaries:
pH: ≦5.0, >5.0 to ≦6.0, >6.0 to ≦7.0, and >7.0
glucose, total protein, ketones, total bilirubin, nitrite, RBC (erythrocytes), and urobilinogen: negative and other (consisting of ‘trace’, ‘positive’, ‘+’, ‘++’, ‘+++’, or ‘++++’)
WBC (leukocytes): negative, positive

The overall incidence of post-Baseline marked abnormalities for hematology and chemistry parameters is also presented. Where applicable, the incidence of post-Baseline marked abnormalities is presented separately by whether the value was abnormally high or low. Any post-Baseline markedly abnormal laboratory measurement collected after the first dose of cilansetron and within 30 days of the last dose of cilansetron was included. However, assessments which were done more than 30 days after last subject visit and after database lock were not included in the analysis. Subjects were counted at most once for the post-Baseline marked abnormality for each laboratory parameter. Laboratory ranges used to identify post-Baseline markedly abnormal results are presented in Table 5.

TABLE 5
Laboratory Ranges Used to Identify Markedly Abnormal Results

| Laboratory Parameter | Lower Limit | Upper Limit |
|---|---|---|
| Hematology: | | |
| Hemoglobin (g/dL) | | |
| Male | <11.5 | NA |
| Female | <9.5 | NA |
| Hematocrit (%) | | |
| Male | <37 | NA |
| Female | <32 | NA |
| RBC (×10⁶/μL) | | |
| Male | <2.5 | NA |
| Female | <2.0 | NA |
| WBC (×10³/μL) | <2.80 | >16.00 |
| Eosinophils (%) | NA | >10 |
| Basophils (%) | NA | >15 |
| Lymphocytes (%) | NA | >80 |
| Monocytes (%) | NA | >40 |
| Neutrophils (Total) (%) | <15 | NA |
| Platelet Count (×10³/μL) | <75 | >700 |
| Chemistry: | | |
| AP (U/L) | NA | >390 |
| AST (SGOT) (U/L) | NA | >150 |
| ALT (SGPT) (U/L) | NA | >165 |
| GGT (U/L) | | |
| Male | NA | >100 |
| Female | NA | >90 |
| Glucose (mg/dL) | <30 | >250 |
| Total Bilirubin (mg/dL) | NA | >2.0 |
| Creatinine (mg/dL) | NA | >2.0 |
| Sodium (mmol/L) | <120 | >165 |
| Potassium (mmol/L) | <2.5 | >6.5 |
| Lactic dehydrogenase (LDH) (U/L) | NA | >3.0 × upper |
| | | limit of normal |
| Total Protein (g/dL) | <4.5 | NA |
| Uric Acid (mg/dL) | | |
| Male | NA | >10.5 |
| Female | NA | >8.5 |
| CK (U/L) | NA | >3.0 × upper |
| | | limit of normal |
| Triglycerides (mg/dL) | NA | >500 |
| Cholesterol (mg/dL) | NA | >500 |

Note:
NA = Not Applicable.

A listing of all markedly abnormal hematology and blood chemistry values collected within 30 days of discontinuation of study medication is presented; all laboratory values collected after discontinuation of study medication were identified.

Any laboratory measurement collected after the first dose of study medication and within 30 days of the last dose of study medication was included. Subjects were counted at most once for the clinically relevant pre-defined change for each laboratory parameter. The criteria for clinically relevant pre-defined changes are displayed in Table 6.

TABLE 6
Pre-defined Changes for Laboratory Results

Delta

| Laboratory Parameter | Low¹ | High¹ |
|---|---|---|
| Hematology: | | |
| Hemoglobin (g/dL) | 2.0 | 2.0 |
| Hematocrit (%) | 6.0 | 6.0 |
| RBC (×10⁶/μL) | 0.7 | 0.7 |
| WBC (×10³/μL) | 3.0 | 3.0 |
| Eosinophils (%) | 3.1 | 3.1 |
| Basophils (%) | 1.2 | 1.2 |
| Lymphocytes (%) | 14.0 | 14.0 |
| Monocytes (%) | 3.6 | 3.6 |
| Neutrophils (Total) (%) | 17.0 | 17.0 |
| Platelet Count (×10³/μL) | 100.0 | 100.0 |
| Chemistry: | | |
| AP (U/L) | 28.0 | 28.0 |
| AST (SGOT) (U/L) | 22.0 | 22.0 |
| ALT (SGPT) (U/L) | 28.0 | 28.0 |
| GGT (U/L) | 15.0 | 15.0 |
| Glucose (mg/dL) | 32.0 | 32.0 |
| Total Bilirubin (mg/dL) | 0.6 | 0.6 |
| Creatinine (mg/dL) | 0.4 | 0.4 |
| Sodium (mmol/L) | 8.0 | 8.0 |
| Potassium (mmol/L) | 1.0 | 1.0 |
| LDH (U/L) | 50.0 | 50.0 |
| Total Protein (g/dL) | 1.0 | 1.0 |
| Uric Acid (mg/dL) | 1.5 | 1.5 |
| CK (U/L) | 175 | 175 |
| Triglycerides (mg/dL) | 125 | 125 |
| Cholesterol (mg/dL) | 80 | 80 |

¹Predefined change low was defined as a decrease ≧Delta. Predefined change high was defined as an increase ≧Delta.

The overall incidence of post-Baseline marked abnormalities for hematology and chemistry parameters and the overall incidence of clinically relevant re-defined change of hematogy, and chemistry are present overall by gender, age (18-40, 41-64 and greater or equal to 65 years), and country.

Vital Signs

Summary statistics are presented for results at each visit and at Endpoint, as well as for change from Baseline results at each clinic visit and at Endpoint for systolic and diastolic blood pressure, pulse rate, and temperature.

For weight, summary statistics are presented at Baseline and at Endpoint, as well as for the change from Baseline to Endpoint.

For Study 3007, “baseline” was defined as Visit 1 of core Study 3006 for subjects randomized to the cilansetron treatment group in Study 3006 and as the last post-Baseline visit of Study 3006 (normally Visit 5) for subjects randomized to the placebo group in Study 3006. The by-timepoint analyses were based on timepoints defined in Section 5.7.1.2.

The overall incidence of post-Baseline marked abnormalities for vital signs is presented overall and by time interval. Any post-Baseline markedly abnormal vital sign measurement collected after the first dose of study medication and within 30 days of the last dose of study medication was included. Subjects were counted at most once for the post-Baseline marked abnormality for each vital signs parameter. A listing of post-Baseline markedly abnormal vital signs is presented; all measurements obtained after discontinuation of study medication were identified. Criteria used to define post-Baseline marked abnormalities are presented in Table 7. Any vital sign outside the cutpoints or which represented a pre-defined change with respect to the Baseline value was considered as markedly abnormal.

TABLE 7
Vital Sign Ranges Used to Identify Markedly Abnormal Results

| Vital Sign | Cutpoints | Pre-defined change |
|---|---|---|
| Systolic Blood Pressure | ≧180 mmHg | Increase of ≧20 mmHg |
| | ≦90 mmHg | Decrease of ≧20 mmHg |
| Diastolic Blood Pressure | ≧105 mmHg | Increase of ≧15 mmHg |
| | ≦50 mmHg | Decrease of ≧15 mmHg |
| Pulse rate | ≧120 bpm | Increase of ≧15 bpm |
| | ≦50 bpm | Decrease of ≧15 bpm |
| Weight | NA | Increase of ≧7% |
| | NA | Decrease of ≧7% |

Note:
BPM = beats per minute

Electrocardiograms

All analyses and summaries for ECG assessments were based on the evaluation performed in the central ECG laboratory.

For Study 3007, “baseline’ was defined as the pre-treatment period of core Study 3006 for subjects randomized to the cilansetron treatment group in Study 3006 and as the last post-Baseline visit of Study 3006 (normally Visit 5) for subjects randomized to the placebo group in Study 3006. The by-timepoint analyses were based on timepoints defined in Section 5.7.1.2.

Summary statistics are presented for PR, QRS, QT, and QT_{c} intervals (Bazett and Fridericia formulas) and heart rate at each visit and at Endpoint, as well as for change from Baseline results at each post-Baseline visit and at Endpoint. Bazett's and Fridericia's QT_{c} intervals were calculated based on the following formulas:
Bazett: QT_{c} intervals=QT/√RR
Fridericia: QT_{c} intervals=QT/³√RR

Markedly abnormal ECG parameter values collected after the first dose of study medication and within 30 days of the last dose of study medication were summarized overall. The following criteria were applied to identify markedly abnormal parameter values:
PR ≧0.210 sec;
QRS ≧0.120 sec;
QT_{c} >0.440 sec;

Heart rate ≧120 bpm or ≦50 bpm or change of ≧15 bpm.

The determination of markedly abnormal for QT_{c} was carried out separately for both the Bazett correction and the Fridericia correction.

The incidence of treatment-emergent ECG abnormalities is presented overall and by type of abnormality. A treatment-emergent ECG abnormality was an abnormality identified by the ECG core laboratory that was not present at baseline (e.g., prior to the entry into core Study 3006), and was observed on or after initiation of study medication and up to 30 days after discontinuation of study medication. The incidence of treatment-emergent ECG abnormalities is presented by time interval, as well as overall for the entire treatment period. The incidence of treatment-emergent abnormalities is also presented in more general categories (e.g., presenting incidence of treatment-emergent myocardial infarction overall as well as by location), depending on whether it mitigates the interpretation.

A listing of markedly abnormal ECG parameter values and a listing of treatment-emergent ECG abnormalities are presented.

The overall incidence of marked abnormalities for ECG parameters is presented overall by gender and overall.

Physical Examinations

Shifts from Baseline to Endpoint (normal, abnormal, not done) are presented for the following sites/systems examined:
HEENT
Cardiovascular
Respiratory
Lymphatics
Abdominal
Musculoskeletal/connective tissue
Neurologic/psychiatric
Dermatologic
General nutritional
Urogenital
Rectal examination
   Quality of Life Analysis

Mean changes from Baseline for the IBS-QOL total score and subscale domains were summarized via descriptive statistics at Visit 3 (Month 3) and Visit 6 (Month 12) of Study 3007 for subjects randomized to the placebo group in core Study 3006 (or for subjects randomized to the cilansetron treatment group in the core Study 3006, at Visit 5 (Month 3), at Visit 3 (Month 6) and Visit 6 (Month 15) of Study 3007).

For Study 3007, “baseline” was defined as the last post-Baseline visit of Study 3006 (normally Visit 5) for subjects randomized to the placebo group and as Visit 1 of core Study 3006 for subjects randomized to the cilansetron treatment group.

The overall IBS-QOL score and the separate IBS-QOL domain scores (i.e. dysphoria, interference with activity, body image, health worry, food avoidance, social reaction, sexual, and relationship) were summarized by treatment group using descriptive statistics. The calculation of each domain score and the overall score is detailed in the user's manual. For each of the domain scores, as well as for the overall score, the change from Baseline to Endpoint was summarized descriptively. Based on the instruction from the author of the IBS-QOL questionnaire, the missing data of the IBS-QOL questionnaire were handled as follows:
If less than or equal to 20% of the items within a subscore (or total score) were missing for a subject, the subscore (or total score) was the summation of the non-missing scores for that subject;
If more than 20% of the items within a subscore (or total score) were missing, the subscore (or total score) was missing for that subject.
   Determination of Sample Size

For Study 3007, the number of subjects was determined solely by the number of completers from the core efficacy protocols. Assuming 680 subjects were randomized into each core efficacy protocol and a dropout rate of 25%, approximately 510 subjects completed each core efficacy protocol. Assuming a further 25% of subjects who completed each core efficacy protocol did not enroll into the 52-week extension study, a total of 383 subjects per each core efficacy protocol were expected to be enrolled into the 52-week extension study.

For Study 3008, approximately 800 subjects were needed to complete 52 weeks of treatment. Assuming a 50% dropout rate, a sample size of 1600 was needed to achieve the planned level of completed subjects.

Study Subjects

For Study 3007, subjects who were randomized to cilansetron 2 mg TID in Study 3006 and continued cilansetron 2 mg TID during Study 3007 will be referred to as C/C subjects or C/C treatment group; subjects who were randomized to placebo in Study 3006 and were taking cilansetron 2 mg TID during Study 3007 will be referred to as P/C subjects or P/C treatment group in this report.

Disposition of Subjects

Overall subject disposition for subjects who enrolled in Study 3007 is summarized in Table 8.

TABLE 8
Subject Disposition (All Subjects Enrolled) - 3007

| Parameter | Statistic | C/C | P/C | Overall |
|---|---|---|---|---|
| Number of subjects enrolled^{a} | n | 262 | 281 | 543 |
| Number of subjects who received study medication | n (%) | 262 (100) | 281 (100) | 543 (100) |
| Number of subjects in the Safety population^{b} | n (%) | 257 (98) | 279 (>99) | 536 (99) |
| Number of subjects who completed the study | n (%) | 128 (49) | 135 (48) | 263 (48) |
| Number of subjects who withdrew from the study | n (%) | 134 (51) | 146 (52) | 280 (52) |
| Primary reason for withdrawal: | | | | |
| Adverse event and lack of efficacy | n (%) | 23 (9) | 34 (12) | 57 (10) |
| Baseline complaint/adverse event | n (%) | 10 (4) | 14 (5) | 24 (4) |
| Lack of efficacy | n (%) | 20 (8) | 25 (9) | 45 (8) |
| Protocol violation | n (%) | 3 (1) | 6 (2) | 9 (2) |
| Administrative reason | n (%) | 31 (12) | 27 (10) | 58 (11) |
| Other | n (%) | 47 (18) | 40 (14) | 87 (16) |
| Number of subjects who died | n (%) | 0 | 0 | 0 |
| IBS severity at the 3006 screening | | | | |
| Severe (Interruption of Activities score of ≧3) | n (%) | 147 (56) | 152 (54) | 299 (55) |
| Non-severe | n (%) | 110 (42) | 127 (45) | 237 (44) |
| Number of subjects continued after Protocol | n (%) | 39 (15) | 43 (15) | 82 (15) |
| Amendments 4 and 5 | | | | |
| Number of subjects discontinued due to Protocol | n (%) | 39 (15) | 30 (11) | 69 (13) |
| Amendments 4 and 5 | | | | |
| Reason for discontinuation^{c} | | | | |
| Informed consent form not signed | n (%) | 9 (3) | 11 (4) | 20 (4) |
| Subject did not agree to continue | n (%) | 32 (12) | 24 (9) | 56 (10) |
| Rating <3 for Interruption of Activities | n (%) | 21 (8) | 19 (7) | 40 (7) |
| Subject did not exhibit severe IBS symptoms | n (%) | 11 (4) | 8 (3) | 19 (3) |
| Subject did not benefit from treatment | n (%) | 4 (2) | 1 (<1) | 5 (1) |

C = cilansetron 2 mg TID;
P = placebo
^{a}All subjects who signed an informed consent are considered as enrolled.
^{b}The Safety population is defined as all subjects who completed Study 3006 and continued into Study 3007, received at least one dose of cilansetron in Study 3007, and had at least one assessment of safety after starting cilansetron in Study 3007.
^{c}Multiple reasons can be applicable to the same subjects.
Note:
Percentages are based on the number of subjects who received study medication.

A total of 587 subjects completed the 12-week core efficacy Study 3006, 283 subjects in the cilansetron 2 mg TID group versus 304 subjects in the placebo group. Of these, 543 subjects (262 subjects versus 281 subjects, respectively) were enrolled and received study medication in Study 3007. Of the 543 subjects who received study medication, 263 subjects (48%) completed the study and 280 subjects (52%) withdrew from the study. The completers included 20 subjects who were terminated up to two weeks early at the end of the study at the Sponsor's request, in order to accelerate report production and include a final clinical study report in the 120-day update to the NDA submission.

The most common primary reason for withdrawal was lack of efficacy (combined categories of “lack of efficacy” and “AE and lack of efficacy”, 18%) followed by the category of “other” (16%), AE (combined categories of “AE and lack of efficacy” and “Baseline complaint/AE”, 15%), administrative reason (11%), and protocol violation (2%). Most subjects in the category “other” withdrew consent or were lost to follow-up as per Investigator comments on the Termination CRF. In addition to the 81 subjects (15%) who discontinued due to AE per Termination CRF, four subjects discontinued study medication due to AEs per AE CRF, but had the primary reason for early termination recorded on the Termination CRF as lack of efficacy, protocol violation, and administrative reason.

Small differences were observed between C/C and P/C subjects for the individual reasons of AE (13% versus 17%), lack of efficacy (17% versus 21%), and administrative reason (18% versus 14%, respectively). A possible explanation for the difference in discontinuations due to AE and lack of efficacy is that subjects who started cilansetron treatment in Study 3006 and discontinued during the first 12 weeks of treatment are not accounted for in this analysis. The difference in discontinuations due to administrative reason is driven by the difference in discontinuations due to Protocol Amendments 4 and 5 (see discussion below).

The overall withdrawal rate decreased from 21% during the first three months of Study 3007 to 13% during the last three months of Study 3007 in both C/C and P/C subjects. Note the drop in the number of subjects in the fifth (>12 months) interval and the small number of P/C subjects (55) relative to C/C subjects (147) in this interval, which limits the value of comparisons of safety data between C/C subjects and P/C subjects in this time interval.

On 19 Dec. 2003, the FDA required that only severe diarrhea-predominant IBS subjects be included in this study. The protocol was amended twice to address the partial clinical hold by the FDA. Amendment 4 allowed subjects with non-severe IBS to continue at the Investigator's discretion. Amendment 5 allowed only severe IBS subjects (Interruption of Activities score of 3 or 4 at the start of Study 3006) to continue regardless of the Investigator's opinion. Investigators were to check Administrative reason on the Termination CRF for subjects who were discontinued due to Amendments 4 or 5. Of note, 299 of 543 enrolled subjects (55%) had severe IBS. Of the 151 subjects (28%) who were ongoing at the time, 69 subjects (13% of enrolled subjects) were discontinued due to one or more of the following reasons related to Amendments 4 or 5: subject did not agree to continue (10%), score of <3 for Interruption of Activities (7%), informed consent form to continue study not signed (4%), subject did not exhibit severe IBS symptoms (3%), and subject did not benefit from treatment (1%). Due to the relatively small (13%) proportion of subjects who were discontinued as a result of the partial clinical hold at a relatively late stage of the study, data presented in this report are mostly reflective of all severities of IBS and not only severe. Overall subject disposition for subjects who enrolled in Study 3008 is summarized in Table 9:

TABLE 9
Subject Disposition (All Subjects Enrolled) - 3008

[symbol]

| | | Cilansetron | |
|---|---|---|---|
| Parameter | Statistic | 2 mg TID | |
| Number of subjects enrolled | n | 1606 | |
| Number of subjects who received study | n (%) | 1457 | (91) |
| medication^{a} | | | |
| Number of subjects in the Safety population | n (%) | 1454 | (>99) |
| Number of subjects who completed the study | n (%) | 1071 | (74) |
| Number of subjects who withdrew from the study | n (%) | 386 | (26) |
| Primary reason for withdrawal: | | | |
| Adverse event and lack of efficacy | n (%) | 119 | (8) |
| Baseline complaint/adverse event | n (%) | 75 | (5) |
| Lack of efficacy | n (%) | 55 | (4) |
| Protocol violation | n (%) | 32 | (2) |
| Administrative reason | n (%) | 4 | (<1) |
| Other | n (%) | 101 | (7) |
| Number of subjects who died | n (%) | 1 | (<1) |

^{a}Percentages are based on the number of subjects enrolled.
Note:
All subjects who signed an informed consent are considered as enrolled.
Note:
Percentages are based on the number of subjects who received study medication unless otherwise specified.
Note:
Two subjects (115017 and 160003) withdrew from the study due to AEs that were not treatment-emergent,therefore these subjects and events are not included in the TEAE table summarizing discontinuation due to AEs
Data source: Table 10.1.1.1.1

Of the 1606 subjects who were enrolled (i.e., signed an informed consent), 1457 subjects received study medication. Of the 1457 subjects who received study medication, 1071 subjects (74%) completed the study and 386 subjects (26%) withdrew from the study. The most common primary reasons for withdrawal were AEs (combined categories of “AE and lack of efficacy” and “Baseline complaint/AE”, 13%) and lack of efficacy (combined categories of “lack of efficacy” and “AE and lack of efficacy”, 12%) followed by the categories of “other” (7%), protocol violation (2%), and administrative reason (<1%). Most subjects in the category “other” withdrew consent.

The overall withdrawal rate decreased from the first month to the subsequent months over the first three months of the study (9%, 4%, and 4%) and decreased from 16% during the first three month interval to 7% during the second three-month interval and to 3% during both the third and fourth three-month intervals. A total of 18 countries enrolled subjects in this study with 11 countries enrolling at least 5% of the subjects: Poland (13%), Ukraine (12%), India (11%), Estonia (8%), Russia (7%), Slovakia (7%), Australia (6%), Czechoslovakia (6%), Israel (6%), South Africa (6%) and New Zealand (5%). None of the individual study sites enrolled more than 4% of the subjects.

Efficacy Evaluation

Among other findings (discussed more fully below), it was determined in the 3007 Study that cilansetron 2 mg TID is generally safe, well-tolerated, and improved QOL in the study population of the 3007 Study over 52 weeks of treatment. In particular, for example, it was determined in the 3007 Study that: (i) the beneficial effect of cilansetron 2 mg TID on the QOL observed during Study 3006 was maintained during Study 3007 in C/C subjects and an improvement was observed in P/C subjects after the start of cilansetron treatment, (ii) from the start of cilansetron treatment, greater improvement was observed in the C/C group compared with the P/C group, perhaps due to a placebo effect in the P/C group at Baseline, (iii) subdomains of QoL with the greatest improvements included those with the lowest observed baseline values: interference with activity score, food avoidance score, and dysphoria score, (iv) the only severe TEAEs reported for ≧1% of subjects overall were constipation (3%), abdominal pain NOS (2%), and headache NOS (1%), (v) the prevalence of GI disorders and constipation decreased over time and decreasing trends were observed for each of the most common individual TEAEs, (vi) other clinical areas of attention included seizure/convulsion and epilepsy; syncope/loss of consciousness; deep venous thrombosis; cardiac disorders; and drug interaction with fluvoxamine, (vii) treatment-emergent SAEs of cardiac disorders were reported for four subjects (supraventricular tachycardia, myocardial infarction, and two cases of angina pectoris), (viii) of the most common TEAEs, higher incidence was noted among female subjects compared with male subjects for constipation (see above), diarrhea excluding infective, nausea and vomiting symptoms, sinusitis NOS, and urinary tract infections; in contrast, blood CK increased was more common in male subjects compared with female subjects, (ix) the incidence increased with age for constipation and a similar but less apparent trend was seen for abdominal pain NOS and flatulence, bloating and distension; in contrast, the incidence decreased with age for nausea and vomiting symptoms, sinusitis NOS, and a similar trend was seen for lower respiratory tract and lung infections and headache NOS, (x) the most common post-Baseline laboratory abnormalities that met markedly abnormal criteria were markedly high GGT (overall: 6%, males: 7%; females: 5%), markedly high triglycerides (3%), markedly high CK (3%), markedly high uric acid (1%), and markedly high eosinophils (six subjects, 1%), and (xi) treatment-emergent ECG abnormalities with an incidence ≧5% included sinus arrhythmia (21%), poor R-wave progression (11%), sinus bradycardia (10%), early repolarization-normal variant (8%), non-specific T-wave changes (7%), borderline LA enlargement (6%), and early R-wave transition (6%).

Among other findings (discussed more fully below), it was determined in the 3007 Study that cilansetron 2 mg TID is generally safe, well-tolerated, and improved QOL in the study population of the 3007 Study over 52 weeks of treatment. In particular, for example, it was determined in the 3007 Study that: (i) the most frequently reported events were GI disorders including constipation, GI and abdominal pains (excluding oral and throat), upper respiratory tract infections (pathogen class unspecified), headaches NOS, nausea and vomiting symptoms, and diarrhea (excluding infective), (ii) the prevalence of GI disorders and constipation decreased over time mostly from the first three-month interval to the second three-month interval of the study and remained stable through the remainder of the study, (iii) decreasing trends were observed for each of the most common individual TEAEs except for headache, for which the prevalence remained stable over time, (iv) of the most common TEAEs, constipation (15%) and nausea (5%) were more frequently reported among females compared with males (constipation: 19% versus 11%; nausea: 7% versus 3%), (v) the most common (>10%) ECG abnormalities were sinus bradycardia, sinus arrhythmia, and early-repolarization-normal variant, and (vi) the incidence of treatment-emergent markedly abnormal QTc(B) was 8% (105 subjects) and the incidence of treatment-emergent markedly abnormal QTc(F) was 2% (30 subjects)

Demographic data are summarized in Table 10.

TABLE 10
Subject Demographics (Safety Population)

| | | C/C | P/C | Overall |
|---|---|---|---|---|
| Parameter | Statistic | (N = 257) | (N = 279) | (N = 536) |
| Age (years) | n | 257 | 279 | 536 |
| | Mean (S.E) | 48.9 (0.8) | 49.9 (0.8) | 49.4 (0.6) |
| | Median | 49 | 50 | 50 |
| | Min-Max | 18-86 | 18-79 | 18-86 |
| Age category (years) | n | 257 | 279 | 536 |
| 18-40 | n (%) | 72 (28) | 67 (24) | 139 (26) |
| 41-64 | n (%) | 152 (59) | 178 (64) | 330 (62) |
| ≧65 | n (%) | 33 (13) | 34 (12) | 67 (13) |
| Gender | n | 257 | 279 | 536 |
| Male | n (%) | 89 (35) | 88 (32) | 177 (33) |
| Female | n (%) | 168 (65) | 191 (68) | 359 (67) |
| Race | n | 257 | 279 | 536 |
| Caucasian | n (%) | 242 (94) | 265 (95) | 507 (95) |
| Black | n (%) | 3 (1) | 8 (3) | 11 (2) |
| Asian | n (%) | 3 (1) | 0 | 3 (<1) |
| Other | n (%) | 9 (4) | 6 (2) | 15 (3) |
| Current use of tobacco products | n | 257 | 279 | 536 |
| Yes | n (%) | 48 (19) | 52 (19) | 100 (19) |
| No | n (%) | 209 (81) | 227 (81) | 436 (81) |
| Prior laxative use | n | 257 | 279 | 536 |
| Yes | n (%) | 3 (1) | 3 (1) | 6 (1) |
| No | n (%) | 254 (99) | 276 (99) | 530 (99) |
| Laxative use | n | 257 | 279 | 536 |
| Never | n (%) | 254 (99) | 276 (99) | 530 (99) |
| Rarely (1 to 3 times) | n (%) | 3 (1) | 1 (<1) | 4 (<1) |
| Occasionally (4 to 6 times) | n (%) | 0 | 2 (<1) | 2 (<1) |
| Frequently (7 to 12 times) | n (%) | 0 | 0 | 0 |
| Regularly (>12 times) | n (%) | 0 | 0 | 0 |

C = cilansetron 2 mg TID;
P = placebo
Note:
Data presented in this table are from the pretreatment period of Study 3006.
Note:
Percentages are based on the number of subjects in the Safety Population with a response for each assessment.

The age of the subjects ranged from 18 years to 86 years and the mean age was 49.4 years. Most subjects were in the 41 to 64 years age category (62%) followed by the 18 to 40 years category (26%), while only 13% of subjects were 65 years or older. Most subjects were female (67%). The majority of subjects were Caucasian (95%) followed by the categories of “other” (3%), Black (2%), and Asian (<1%). Nineteen percent of the subjects were using tobacco products and only 1% of subjects had a history of prior laxative use. No relevant difference was observed between C/C and P/C subjects for any of the demographic parameters.

Other Baseline Characteristics

The mean height was 1.69 m, the mean weight was 79.3 kg, and the mean BMI was −27.73 kg/m² with 34% of subjects in the ≧30 kg/m² BMI category. Baseline mean systolic blood pressure was 123.6 mmHg, diastolic blood pressure was 77.0 mmHg, pulse rate was 72.1 bpm, and Baseline mean body temperature was 36.61° C. No relevant difference was observed between C/C and P/C subjects for any of the parameters.

Most subjects thought their IBS symptoms often (more than one-quarter of the time, 26%), very often (more than one half of the time, 29%), or almost always (27%) significantly interfered with their ability to effectively perform their activities. Based on the responses to the Interruption of Activities assessment, 56% of subjects in the Safety population had severe IBS (responses of very often or almost always). No relevant difference was observed between C/C and P/C subjects.

Medical History

The most common (≧10%) medical history conditions included:
surgical and medical procedures of cholecystectomy (23%), hysterectomy (20%), appendectomy (15%), tonsillectomy (15%), and tubal ligation (13%);
GI disorders of hemorrhoids (34%), gastroesophageal reflux disease (24%), dyspepsia (14%), and diverticulum NOS (14%); and
drug hypersensitivity (30%), depression (25%), headache NOS (21%), hypertension NOS (20%), seasonal allergy (19%), anxiety (14%), back pain (12%), hypercholesterolemia (12%), insomnia (11%), osteoarthritis NOS (10%), migraine NOS (10%), and myopia (10%).
   Previous and Concomitant Medication

Most subjects (95%) took at least one concomitant medication with 78% of subjects who took at least one key concomitant medication. Categories of the most frequently used (≧10%) key concomitant medications included other analgesics and antipyretics (39%), antidepressants (28%), laxatives (16%), antihistamines for systemic use (14%), opioids (10%), and plain corticosteroids for systemic use (10%). The WHO generic terms with ≧10% of subjects included paracetamol (19%) and acetylsalicylic acid (15%). No relevant difference was observed between C/C and P/C subjects, except for slightly higher incidences in general for C/C subjects compared with P/C subjects, probably due to the longer exposure in the C/C group. No impact on safety was noted with these medications.

The GERB recommended that fluvoxamine be a prohibited medication for cilansetron studies based on the results of a pharmacokinetic drug interaction study. It was determined that cilansetron plasma levels increased approximately six to seven times when administered concurrently with fluvoxamine. One subject took fluvoxamine while taking cilansetron 2 mg TID.

Efficacy Results

At the start of Study 3007, the mean IBS-QOL total score was higher in C/C subjects (69.5) compared with P/C subjects (62.1), reflecting the beneficial effect of cilansetron 2 mg TID versus placebo during the 12 weeks of Study 3006. Subsequent assessments after three months and 12 months treatment in Study 3007 did not reveal relevant treatment group differences in the mean IBS-QOL total score. At Endpoint, the overall mean IBS-QOL total score was 70.8 (70.7 in C/C subjects compared with 70.9 in P/C subjects).

“Baseline”, in Study 3007, was defined as Day 1 (Visit 2) of core Study 3006 for C/C subjects and Day 1 (Visit 1) of the extension Study 3007 for P/C subjects, which corresponded with Day 91 (Visit 5) of the core Study 3006. At Baseline, the mean IBS-QOL total score was higher in P/C subjects (62.1) compared with C/C subjects (50.5), reflecting the effect of the 12-week placebo treatment in P/C subjects versus the treatment-naïve C/C subjects. At Endpoint, C/C subjects had a greater improvement (20.1) from Baseline compared with P/C subjects (8.9), which was probably due to the Baseline differences described above rather than the three-month difference in the length of exposure to cilansetron 2 mg TID.

For the individual subscores, results were similar to those described above for the IBS-QOL total score. Subscores with the greatest improvements during cilansetron treatment included those with the lowest observed Baseline values: interference with activity score, food avoidance score, and dysphoria score.

Overall, the beneficial effect of cilansetron 2 mg TID on the QOL observed during Study 3006 was maintained during Study 3007 in C/C subjects. An improvement was observed in P/C subjects after the start of cilansetron treatment consistent with the therapeutic effect of cilansetron 2 mg TID. Subdomains with the greatest improvements included those with the lowest observed Baseline values: interference with activity score, food avoidance score, and dysphoria score.

5050

At Baseline, the mean IBS-QOL overall score was higher in the cilansetron group (52.6) compared with the placebo group (51.9). Moreover, as is evident in Table 10.5, a significant improvement (p<0.050) from baseline was observed for IBS-QoL total score, interference with activity, body image, sexual score and dysphoria.

TABLE 10.5

| | Cilansetron | Placebo |
|---|---|---|
| Sexual | 72.2 | 73.9 |
| Health Worry | 65.3 | 68.1 |
| Relationship | 62.5 | 60.5 |
| Body Image | 61.7 | 61.8 |
| Social Reaction | 58.3 | 59.7 |
| Dysphoria | 47.4 | 45.3 |
| Interference with activity | 41.1 | 38.7 |
| Food avoidance | 38.3 | 37.6 |

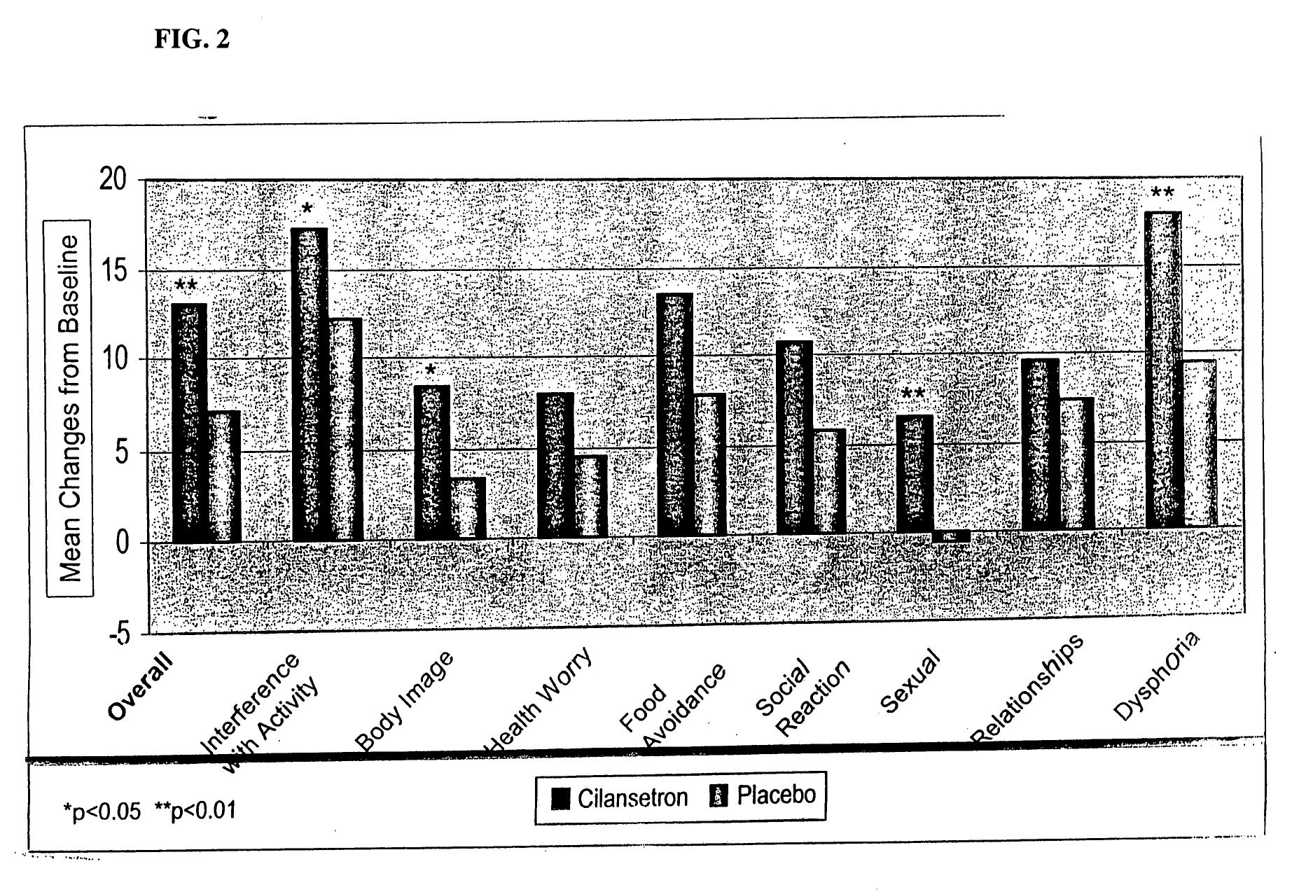

FIG. 2 illustrates the mean change from baseline to end point in each individual subscale score of IBS-QoL for the cilansetron group versus the placebo group.

3008

Of the 1457 subjects who received study medication, three subjects were excluded from the safety population due to lack of post-Baseline safety data. An overall summary of demographic data for the Safety population is displayed in Table 11.

TABLE 11
Subject Demographics (Safety Population) - 3008

| | | Cilansetron 2 mg TID |
|---|---|---|
| Parameter | Statistic | (N = 1454) |
| Age (years) | n | 1454 |
| | Mean (S.E) | 45.6 (0.4) |
| | Median | 46 |
| | Min-Max | 17-84 |
| Age category (years) | n | 1453¹ |
| 18-40 | n (%) | 554 (38) |
| 41-64 | n (%) | 757 (52) |
| ≧65 | n (%) | 142 (10) |
| Gender | n | 1454 |
| Male | n (%) | 650 (45) |
| Female | n (%) | 804 (55) |
| Race | n | 1454 |
| Caucasian | n (%) | 1218 (84) |
| Black | n (%) | 2 (<1) |
| Asian | n (%) | 156 (11) |
| Other | n (%) | 78 (5) |
| Current use of tobacco products | n | 1454 |
| Yes | n (%) | 243 (17) |
| No | n (%) | 1211 (83) |
| Prior laxative use | n | 1454 |
| Yes | n (%) | 53 (4) |
| No | n (%) | 1401 (96) |
| Laxative use | n | 1454 |
| Never | n (%) | 1401 (96) |
| Rarely (1 to 3 times) | n (%) | 41 (3) |
| Occasionally (4 to 6 times) | n (%) | 12 (<1) |
| Frequently (7 to 12 times) | n (%) | 0 |
| Regularly (>12 times) | n (%) | 0 |

Note:
Percentages are based on the number of subjects in the Safety Population with a response for each assessment.
¹One subject was less than 18 years old (17) and was not counted in any age category.
Data source: Table 10.1.1.6.1

The age of the subjects ranged from 17 years to 84 years and the mean age was 45.6 years. Most subjects were in the 41 to 64 years age category (52%) followed by the 18 to 40 years category (38%), while only 10% of subjects were 65 years or older. The majority of subjects were female (55%). Most of subjects were in the Caucasian category (84%) followed by the category of Asian (11%), .other. (5%), and Black (<1%). Seventeen percent of the subjects were using tobacco products at Baseline and 4% of subjects had a history of prior laxative use.

Other Baseline Characteristics

The mean height at baseline was 1.68 m, the mean weight was 72.5 kg, and the mean BMI was 25.55 kg/m2 with 16% of subjects in the ≧30 kg/m2 BMI category. Baseline mean systolic blood pressure was 125.7 mmHg, diastolic blood pressure was 78.7 mmHg, pulse rate was 72.8 bpm, and Baseline mean body temperature was 36.58° C. Baseline assessment of organic or parasitic diseases is presented in ˜xr54i. Abnormal or positive results were obtained in ≦2 subjects for each test (parasites, ova, and stool culture).

Of the 1453 subjects who had colonoscopy done at Baseline, minor abnormalities, which did not exclude the subjects from participation in the study, were revealed in 11% of subjects and were hemorrhoids, diverticulosis, or polyps in most cases.

Most subjects thought their IBS symptoms often (more than one-quarter of the time, 38%) or very often (more than one half of the time, 29%) significantly interfered with their ability to effectively perform their activities. Based on the responses to the interruption of activities assessment, most subjects had IBS of either moderate severity (responses of occasionally or often, 52%) or severe (responses of very often or almost always, 45%).

The duration of IBS ranged from 6 months to 720 months (60 years) and the mean duration of IBS was 92.2 months (7.7 years). Approximately half of the subjects had the following diarrhea related symptoms almost always or very often (more than half of the time) during the six months prior to enrollment: more than three bowel movements a day (56%), loose, mushy, or watery bowel movements (66%), and urgency (47%). Some subjects also experienced the following constipation-related symptoms occasionally or more frequently: fewer than three bowel movements per week (7%), lumpy or hard bowel movements (14%), and need to strain (21%).

Medical History

The most common (≧5%) medical history conditions included:
surgical and medical procedures of cholecystectomy (13%), appendectomy (13%), and hysterectomy (8%),
GI disorders of hemorrhoids (11%), gastritis NOS (7%), and GERD (5%); and
back pain (6%), hypertension NOS (15%), headache NOS (5%), depression (5%), and drug hypersensitivity (5%).
   Previous and Concomitant Medication

Most subjects (66%) took at least one concomitant medication. Categories of the most frequently used (≧5%) concomitant medications included other analgesics and antipyretics (20%), nonsteroidal antiinflammatory/antirheumatic products (13%), drugs for treatment of peptic ulcer (13%), beta blocking agents (11%), topical products for joint and muscular pain (10%), stomatological preparations (8%), antiinflammatory agents (8%), beta-lactam antibacterials and penicillins (8%); decongestants and other nasal preparations for topical use (8%), ACE-inhibitors (7%), antihistamines for systemic use (7%), hormonal contraceptives for systemic use (7%), antidepressants (7%), corticosteroids (7%), laxatives (7%), antiinfectives (7%), other antiasthmatics and inhalants (6%), anxiolytics (6%), antithrombotic agents (6%), opioids (6%), cholesterol and triglyceride reducers (6%), corticosteroids for systemic use (5%), synthetic antispasm and other anticholinergic agents (5%), all other therapeutic products (5%), hormones and related agents (5%), other gynecologicals (5%), and estrogens (5%).

The WHO generic terms with ≧5% of subjects in either group included paracetamol (12%), acetylsalicylic acid (5%), and omeprazole (5%). No impact on safety was noted with these medications.

Safety Evaluation

a. 3007

Extent of Exposure

The extent of exposure to cilansetron 2 mg TID during Studies 3006 and 3007 is summarized in Table 12.

TABLE 12
Exposure to Study Medication (Safety Population)

| | | C/C | P/C | Overall |
|---|---|---|---|---|
| Parameter | Statistic | (N = 257) | (N = 279) | (N = 536) |
| Exposure | n | 244 | 262 | 506 |
| to study | Mean (SE) | 351.2 (8.4) | 254.4 (8.4) | 301.1 (6.3) |
| medication | Median | 445 | 351 | 363 |
| (days) | Min-Max | 90-510 | 2-386 | 2-510 |
| Category of | n | 244 | 262 | 506 |
| exposure | | | | |
| >0-1 month | n (%) | 0 | 33 (13) | 33 (7) |
| >1-2 months | n (%) | 0 | 11 (4) | 11 (2) |
| >2-3 months | n (%) | 2 (<1) | 9 (3) | 11 (2) |
| >3-6 months | n (%) | 49 (20) | 30 (11) | 79 (16) |
| >6-9 months | n (%) | 25 (10) | 25 (10) | 50 (10) |
| >9-12 | n (%) | 22 (9) | 99 (38) | 121 (24) |
| months | | | | |
| >12 months | n (%) | 146 (60) | 55 (21) | 201 (40) |

C = cilansetron 2 mg TID;
P = placebo
Note:
Exposure to study medication is calculated as the date, of first cilansetron dose subtracted from the date of last dose plus 1, without accounting for gaps in study medication use.
Note:
Percentages are based on the total number of subjects in the Safety Population with an evaluation of exposure.

The overall mean duration of exposure to cilansetron 2 mg TID during Studies 3006 and 3007 was 301.1 days (median: 363 days). Subjects in the C/C group had approximately three months longer exposure to cilansetron 2 mg TID compared with P/C subjects (means of 351.2 days versus 254.4 days; medians of 445 days versus 351 days, respectively). Overall, 40% of subjects (60% of C/C subjects versus 21% of P/C subjects) took cilansetron 2 mg TID for more than 12 months. The difference between the C/C and P/C subjects in the duration of exposure to cilansetron 2 mg TID should be considered when interpreting the results of the analyses, e.g., incidences.

In Study 5050, the mean duration of exposure to study medication was 269 days in the cilansetron group compared with 210 days in the placebo group. Overall exposure to study medication exceeded 9 months for 64% of cilansetron-treated patients and 43% of placebo-treated patients.

Drug Holiday

Overall, 26% of subjects had at least one drug holiday with mean total drug holiday duration of 7.23 days, which accounted for 4.9% of the duration of exposure for these subjects. The proportion of subjects with a drug holiday and the total duration of drug holidays were slightly greater for C/C subjects compared with P/C subjects (30% versus 22% and 8.38 days versus 5.85 days, respectively), possibly due to the longer duration of exposure in the C/C group. However, drug holidays accounted for a smaller proportion of the total duration of cilansetron exposure in C/C subjects (3.1%) compared with P/C subjects (7.1%). The proportion of subjects with a drug holiday decreased over time but the decrease by three-month interval was less apparent in C/C subjects (10%, 10%, 11%, 5%, and 9%) compared with P/C subjects (15%, 6%, 5%, 2%, and 0%).

Adverse Events

A summary of TEAEs is presented in Table 13.

TABLE 13
Summary of Treatment-Emergent Adverse Events (Safety Population)

| | | C/C | P/C | Overall |
|---|---|---|---|---|
| Parameter | Statistic | (N = 257) | (N = 279) | (N = 536) |
| Number of subjects with at least one TEAE | n (%) | 220 (86) | 217 (78) | 437 (82) |
| Number of subjects with at least one related TEAE | n (%) | 151 (59) | 150 (54) | 301 (56) |
| Number of subjects with at least one severe TEAE | n (%) | 50 (19) | 44 (16) | 94 (18) |
| Number of deaths | n (%) | 0 | 0 | 0 |
| Number of subjects with at least one SAE | n (%) | 19 (7) | 10 (4) | 29 (5) |
| Number of subjects who permanently discontinued study | n (%) | 33 (13) | 50 (18) | 83 (15) |
| medication due to a TEAE (as reported on the AE CRF) | | | | |
| Number of subjects with at least one TEAE leading to | n (%) | 7 (3) | 4 (1) | 11 (2) |
| dose reduction | | | | |
| Number of subjects with at least one AE of special interest | n (%) | 186 (72) | 185 (66) | 371 (69) |

C = cilansetron 2 mg TID;
P = placebo
Note:
Percentages are based on the total number of subjects in the Safety Population.
Note:
Refer to Section 0 for a definition of TEAEs.
Note:
Related = probably, possibly, or unlikely related; not related = unrelated; unknown = unknown/not applicable.

A total of 82% of subjects experienced at least one TEAE on cilansetron treatment during Studies 3006 and 3007. The overall incidence of TEAEs that were reported as possibly, probably, or unlikely related to study medication was 56%. Severe TEAEs were reported for 18% of subjects. No deaths were reported. Five percent of subjects experienced at least one treatment-emergent SAE. Fifteen percent of subjects experienced TEAEs that led to permanent discontinuation of study medication and 2% of subjects had TEAEs that led to dose reduction. Special interest TEAEs were reported for 69% of subjects. The incidences were slightly higher in general in C/C subjects compared with P/C subjects, possibly due to the longer duration of exposure (e.g., TEAEs, 86% versus 78% and treatment-emergent SAEs, 7% versus 4%, respectively) with the exception of TEAEs that led to discontinuation, which had a lower incidence in C/C subjects (13%) compared with P/C subjects (18%). A possible explanation for this phenomenon is that subjects who started cilansetron treatment in Study 3006 and discontinued during the first 12 weeks of cilansetron treatment are not accounted for in this analysis.

Analysis of Adverse Events

Overall Incidence of Treatment-Emergent Adverse Events

The overall incidence of TEAEs in Study 3007 is presented by primary system organ class, higher level term, and preferred term in Table 14.

Of the 536 subjects in the Safety population, 82% experienced at least one TEAE on cilansetron treatment during Studies 3006 and 3007. The most frequently reported primary system organ classes with events (preferred terms) that occurred in ≧5% of subjects overall included:
GI disorders (50%): constipation (24%), abdominal pain NOS (7%), diarrhea NOS (6%), flatulence (5%), and nausea (5%)
Infections and infestations (32%): sinusitis NOS (7%), upper respiratory tract infection NOS (5%), urinary tract infection NOS (5%), and nasopharyngitis (5%)
Investigations (24%): fecal occult blood positive (6%) and blood CK increased (5%)
Musculoskeletal and connective tissue disorders (16%): incidence for each preferred term was <5% with back pain (3%) as the most common term
Nervous system disorders (12%): headache NOS (6%)

As previously noted, incidences were slightly higher in general in C/C subjects compared with P/C subjects, probably due to the longer duration of exposure in this group.

In Table 14, gender-specific adverse events are denoted by an “*” in the table, and percentages for gender-specific adverse events are based on the number of subjects in the appropriate gender. Percentages for all other adverse events are based on the total number of subjects in the Safety Population. Subjects are counted at most once within each primary system organ class, higher level term, and preferred term. Adverse events were coded to primary system organ class, higher level term, and preferred term using the MedDRA dictionary, version 5.0. All adverse events with onset date on or after the date of first dose of Cilansetron and up through seven days after the last dose of Cilansetron (if not serious) or up through 30 days after the last dose of cilansetron (if serious) will be considered as treatment-emergent. Adverse events which are already present during the pre-treatment period but increase in severity during the treatment period will also be considered as treatment-emergent. Primary system organ class, higher level term, and preferred term are sorted by decreasing frequency in the overall column.

TABLE 14
Overall Incidence of Treatment-Emergent Adverse Events by Primary System Organ Class,
Higher Level Term, and Preferred Term (Safety Population)

| Primary System Organ Class/ | | Treatment Group | | |
|---|---|---|---|---|
| Higher Level Term/ | | Cilansetron 2 mg/ | Placebo/ | |
| Preferred Term | Statistic | Cilansetron 2 mg | Cilansetron 2 mg | Overall |
| Total Number of Subjects in the Safety Population | N | 257 | 279 | 536 |
| Total Number of Male Subjects in the Safety Population | n | 89 | 88 | 177 |
| Total Number of Female Subjects in the Safety Population | n | 168 | 191 | 359 |
| Total Number of Subjects with at Least One TEAE | n (%) | 220 (86%) | 217 (78%) | 437 (82%) |
| Gastrointestinal Disorders | n (%) | 134 (52%) | 134 (48%) | 268 (50%) |
| Gastrointestinal Atonic and Hypomotility Disorders NEC | n (%) | 70 (27%) | 63 (23%) | 133 (25%) |
| Constipation | n (%) | 66 (26%) | 62 (22%) | 128 (24%) |
| Gastrooesophageal Reflux Disease | n (%) | 3 (1%) | 1 (<1%) | 4 (<1%) |
| Oesophageal Reflux Aggravated | n (%) | 3 (1%) | 0 | 3 (<1%) |
| Gastrointestinal and Abdominal Pains (Excl Oral and Throat) | n (%) | 36 (14%) | 33 (12%) | 69 (13%) |
| Abdominal Pain NOS | n (%) | 14 (5%) | 21 (8%) | 35 (7%) |
| Abdominal Tenderness | n (%) | 9 (4%) | 4 (1%) | 13 (2%) |
| Abdominal Pain Upper | n (%) | 7 (3%) | 4 (1%) | 11 (2%) |
| Abdominal Pain Lower | n (%) | 4 (2%) | 6 (2%) | 10 (2%) |
| Abdominal Pain Aggravated | n (%) | 4 (2%) | 0 | 4 (<1%) |
| Abdominal Rebound Tenderness | n (%) | 1 (<1%) | 0 | 1 (<1%) |
| Diarrhoea (Excl Infective) | n (%) | 24 (9%) | 15 (5%) | 39 (7%) |
| Diarrhoea NOS | n (%) | 22 (9%) | 9 (3%) | 31 (6%) |
| Diarrhoea Aggravated | n (%) | 3 (1%) | 2 (<1%) | 5 (<1%) |
| Faecal Incontinence | n (%) | 1 (<1%) | 3 (1%) | 4 (<1%) |
| Diarrhoea Haemorrhagic | n (%) | 0 | 2 (<1%) | 2 (<1%) |
| Flatulence, Bloating and Distension | n (%) | 23 (9%) | 13 (5%) | 36 (7%) |
| Flatulence | n (%) | 16 (6%) | 10 (4%) | 26 (5%) |
| Abdominal Distension | n (%) | 9 (4%) | 6 (2%) | 15 (3%) |
| Nausea and Vomiting Symptoms | n (%) | 19 (7%) | 14 (5%) | 33 (6%) |
| Nausea | n (%) | 15 (6%) | 10 (4%) | 25 (5%) |
| Vomiting NOS | n (%) | 9 (4%) | 9 (3%) | 18 (3%) |
| Dyspeptic Signs and Symptoms | n (%) | 10 (4%) | 7 (3%) | 17 (3%) |
| Dyspepsia | n (%) | 6 (2%) | 7 (3%) | 13 (2%) |
| Dyspepsia Aggravated | n (%) | 2 (<1%) | 0 | 2 (<1%) |
| Eructation | n (%) | 2 (<1%) | 0 | 2 (<1%) |
| Gastric Irritation | n (%) | 1 (<1%) | 0 | 1 (<1%) |
| Haemorrhoids and Gastrointestinal Varices (Excl Oesophageal) | n (%) | 6 (2%) | 9 (3%) | 15 (3%) |
| Haemorrhoids | n (%) | 4 (2%) | 7 (3%) | 11 (2%) |
| Haemorrhoidal Haemorrhage | n (%) | 2 (<1%) | 2 (<1%) | 4 (<1%) |
| Haemorrhoids Aggravated | n (%) | 0 | 1 (<1%) | 1 (<1%) |
| Faeces Abnormal | n (%) | 2 (<1%) | 7 (3%) | 9 (2%) |
| Faeces Hard | n (%) | 1 (<1%) | 5 (2%) | 6 (1%) |
| Faecal Abnormality NOS | n (%) | 1 (<1%) | 1 (<1%) | 2 (<1%) |
| Faecal Impaction | n (%) | 0 | 1 (<1%) | 1 (<1%) |
| Intestinal Haemorrhages | n (%) | 4 (2%) | 4 (1%) | 8 (1%) |
| Rectal Haemorrhage | n (%) | 4 (2%) | 4 (1%) | 8 (1%) |
| Gastrointestinal Spastic and Hypermotility Disorders | n (%) | 5 (2%) | 0 | 5 (<1%) |
| Irritable Bowel Syndrome Aggravated | n (%) | 3 (1%) | 0 | 3 (<1%) |
| Frequent Bowel Movements | n (%) | 1 (<1%) | 0 | 1 (<1%) |
| Irritable Bowel Syndrome | n (%) | 1 (<1%) | 0 | 1 (<1%) |
| Anal and Rectal Pains | n (%) | 0 | 4 (1%) | 4 (<1%) |
| Proctalgia | n (%) | 0 | 4 (1%) | 4 (<1%) |
| Anal and Rectal Signs and Symptoms | n (%) | 1 (<1%) | 3 (1%) | 4 (<1%) |
| Rectal Tenesmus | n (%) | 0 | 2 (<1%) | 2 (<1%) |
| Pruritus Ani | n (%) | 0 | 1 (<1%) | 1 (<1%) |
| Rectal Cramps | n (%) | 1 (<1%) | 0 | 1 (<1%) |
| Gastrointestinal Dyskinetic Disorders | n (%) | 2 (<1%) | 2 (<1%) | 4 (<1%) |
| Gastrointestinal Motility Disorder NOS | n (%) | 2 (<1%) | 1 (<1%) | 3 (<1%) |
| Change in Bowel Habit NOS | n (%) | 0 | 1 (<1%) | 1 (<1%) |
| Gastrointestinal Signs and Symptoms NEC | n (%) | 2 (<1%) | 2 (<1%) | 4 (<1%) |
| Abdominal Discomfort | n (%) | 1 (<1%) | 0 | 1 (<1%) |
| Epigastric Discomfort | n (%) | 0 | 1 (<1%) | 1 (<1%) |
| Halitosis | n (%) | 1 (<1%) | 0 | 1 (<1%) |
| Stomach Discomfort | n (%) | 0 | 1 (<1%) | 1 (<1%) |
| Dental Pain and Sensation Disorders | n (%) | 2 (<1%) | 1 (<1%) | 3 (<1%) |
| Toothache | n (%) | 2 (<1%) | 1 (<1%) | 3 (<1%) |
| Gastritis (Excl Infective) | n (%) | 3 (1%) | 0 | 3 (<1%) |
| Gastritis NOS | n (%) | 3 (1%) | 0 | 3 (<1%) |
| Gastrointestinal Disorders NEC | n (%) | 1 (<1%) | 2 (<1%) | 3 (<1%) |
| Food Poisoning NOS | n (%) | 1 (<1%) | 2 (<1%) | 3 (<1%) |
| Gingival Disorders NEC | n (%) | 1 (<1%) | 2 (<1%) | 3 (<1%) |
| Gingivitis | n (%) | 0 | 2 (<1%) | 2 (<1%) |
| Gingival Atrophy | n (%) | 1 (<1%) | 0 | 1 (<1%) |
| Diaphragmatic Hernias | n (%) | 2 (<1%) | 0 | 2 (<1%) |
| Hiatus Hernia | n (%) | 2 (<1%) | 0 | 2 (<1%) |
| Diverticulum Inflammations | n (%) | 1 (<1%) | 1 (<1%) | 2 (<1%) |
| Diverticulitis NOS | n (%) | 1 (<1%) | 1 (<1%) | 2 (<1%) |
| Gastrointestinal Inflammatory Disorders NEC | n (%) | 1 (<1%) | 1 (<1%) | 2 (<1%) |
| Appendicitis | n (%) | 1 (<1%) | 0 | 1 (<1%) |
| Enteritis | n (%) | 0 | 1 (<1%) | 1 (<1%) |
| Oral Dryness and Saliva Altered | n (%) | 1 (<1%) | 1 (<1%) | 2 (<1%) |
| Dry Mouth | n (%) | 1 (<1%) | 1 (<1%) | 2 (<1%) |
| Anal and Rectal Disorders NEC | n (%) | 0 | 1 (<1%) | 1 (<1%) |
| Rectal Disorder NOS | n (%) | 0 | 1 (<1%) | 1 (<1%) |
| Colitis (Excl Infective) | n (%) | 0 | 1 (<1%) | 1 (<1%) |
| Colitis Ischaemic | n (%) | 0 | 1 (<1%) | 1 (<1%) |
| Dental Disorders NEC | n (%) | 1 (<1%) | 0 | 1 (<1%) |
| Tooth Disorder NOS | n (%) | 1 (<1%) | 0 | 1 (<1%) |
| Tooth Impacted | n (%) | 1 (<1%) | 0 | 1 (<1%) |
| Gastrointestinal Mucosal Dystrophies and Secretion Disorders | n (%) | 1 (<1%) | 0 | 1 (<1%) |
| Colonic Polyp | n (%) | 1 (<1%) | 0 | 1 (<1%) |
| Gingival Pains | n (%) | 1 (<1%) | 0 | 1 (<1%) |
| Gingival Pain | n (%) | 1 (<1%) | 0 | 1 (<1%) |
| Inguinal Hernias | n (%) | 0 | 1 (<1%) | 1 (<1%) |
| Inguinal Hernia NOS | n (%) | 0 | 1 (<1%) | 1 (<1%) |
| Lip and Palate Disorders NEC | n (%) | 0 | 1 (<1%) | 1 (<1%) |
| Chapped Lips | n (%) | 0 | 1 (<1%) | 1 (<1%) |
| Tongue Signs and Symptoms | n (%) | 1 (<1%) | 0 | 1 (<1%) |
| Tongue Coated | n (%) | 1 (<1%) | 0 | 1 (<1%) |
| Umbilical Hernias | n (%) | 1 (<1%) | 0 | 1 (<1%) |
| Umbilical Hernia NOS | n (%) | 1 (<1%) | 0 | 1 (<1%) |
| Infections and Infestations | n (%) | 106 (41%) | 67 (24%) | 173 (32%) |
| Upper Respiratory Tract Infections - Pathogen Class Unspecified | n (%) | 42 (16%) | 19 (7%) | 61 (11%) |
| Sinusitis NOS | n (%) | 24 (9%) | 13 (5%) | 37 (7%) |
| Upper Respiratory Tract Infection NOS | n (%) | 18 (7%) | 8 (3%) | 26 (5%) |
| Sinusitis Acute NOS | n (%) | 0 | 1 (<1%) | 1 (<1%) |
| Urinary Tract Infections | n (%) | 17 (7%) | 12 (4%) | 29 (5%) |
| Urinary Tract Infection NOS | n (%) | 16 (6%) | 10 (4%) | 26 (5%) |
| Bladder Infection NOS | n (%) | 0 | 1 (<1%) | 1 (<1%) |
| Cystitis NOS | n (%) | 1 (<1%) | 0 | 1 (<1%) |
| Kidney Infection NOS | n (%) | 0 | 1 (<1%) | 1 (<1%) |
| Lower Respiratory Tract and Lung Infections | n (%) | 21 (8%) | 7 (3%) | 28 (5%) |
| Bronchitis NOS | n (%) | 18 (7%) | 5 (2%) | 23 (4%) |
| Bronchitis Acute NOS | n (%) | 2 (<1%) | 1 (<1%) | 3 (<1%) |
| Pneumonia NOS | n (%) | 1 (<1%) | 1 (<1%) | 2 (<1%) |
| Bronchopneumonia NOS | n (%) | 0 | 1 (<1%) | 1 (<1%) |
| Rhinoviral Infections | n (%) | 18 (7%) | 10 (4%) | 28 (5%) |
| Nasopharyngitis | n (%) | 18 (7%) | 10 (4%) | 28 (5%) |
| Influenza Viral Infections | n (%) | 11 (4%) | 7 (3%) | 18 (3%) |
| Influenza | n (%) | 11 (4%) | 7 (3%) | 18 (3%) |
| Viral Infections NEC | n (%) | 7 (3%) | 8 (3%) | 15 (3%) |
| Gastroenteritis Viral NOS | n (%) | 5 (2%) | 6 (2%) | 11 (2%) |
| Viral Infection NOS | n (%) | 2 (<1%) | 2 (<1%) | 4 (<1%) |
| Dental Infections - Pathogen Class Unspecified | n (%) | 9 (4%) | 2 (<1%) | 11 (2%) |
| Tooth Abscess | n (%) | 6 (2%) | 1 (<1%) | 7 (1%) |
| Gingival Abscess | n (%) | 2 (<1%) | 0 | 2 (<1%) |
| Tooth Infection | n (%) | 1 (<1%) | 1 (<1%) | 2 (<1%) |
| Gingivitis Infection NOS | n (%) | 1 (<1%) | 0 | 1 (<1%) |
| Gastrointestinal Infections - Pathogen Class Unspecified | n (%) | 4 (2%) | 5 (2%) | 9 (2%) |
| Gastroenteritis NOS | n (%) | 4 (2%) | 5 (2%) | 9 (2%) |
| Fungal Infections NEC | n (%) | 4 (2%) | 4 (1%) | 8 (1%) |
| Fungal Infection NOS | n (%) | 2 (<1%) | 1 (<1%) | 3 (<1%) |
| Nail Fungal Infection NOS | n (%) | 0 | 2 (<1%) | 2 (<1%) |
| Vaginosis Fungal NOS* | n (%) | 1 (<1%) | 1 (<1%) | 2 (<1%) |
| Skin Fungal Infection NOS | n (%) | 1 (<1%) | 0 | 1 (<1%) |
| Streptococcal Infections | n (%) | 5 (2%) | 3 (1%) | 8 (1%) |
| Pharyngitis Streptococcal | n (%) | 4 (2%) | 3 (1%) | 7 (1%) |
| Streptococcal Infection NOS | n (%) | 1 (<1%) | 0 | 1 (<1%) |
| Ear Infections | n (%) | 2 (<1%) | 3 (1%) | 5 (<1%) |
| Ear Infection NOS | n (%) | 2 (<1%) | 2 (<1%) | 4 (<1%) |
| Otitis Media NOS | n (%) | 0 | 1 (<1%) | 1 (<1%) |
| Herpes Viral Infections | n (%) | 3 (1%) | 2 (<1%) | 5 (<1%) |
| Herpes Simplex | n (%) | 2 (<1%) | 1 (<1%) | 3 (<1%) |
| Herpes Simplex Aggravated | n (%) | 1 (<1%) | 0 | 1 (<1%) |
| Herpes Zoster | n (%) | 0 | 1 (<1%) | 1 (<1%) |
| Candida Infections | n (%) | 2 (<1%) | 2 (<1%) | 4 (<1%) |
| Candidal Infection NOS | n (%) | 1 (<1%) | 1 (<1%) | 2 (<1%) |
| Vaginal Candidiasis* | n (%) | 1 (<1%) | 1 (<1%) | 2 (<1%) |
| Infections NEC | n (%) | 3 (1%) | 1 (<1%) | 4 (<1%) |
| Localised Infection | n (%) | 1 (<1%) | 1 (<1%) | 2 (<1%) |
| Infected Varicose Vein | n (%) | 1 (<1%) | 0 | 1 (<1%) |
| Respiratory Tract Infection NOS | n (%) | 1 (<1%) | 0 | 1 (<1%) |
| Skin Structures and Soft Tissue Infections | n (%) | 1 (<1%) | 2 (<1%) | 3 (<1%) |
| Cellulitis | n (%) | 0 | 1 (<1%) | 1 (<1%) |
| Nail Infection NOS | n (%) | 1 (<1%) | 0 | 1 (<1%) |
| Skin Infection NOS | n (%) | 0 | 1 (<1%) | 1 (<1%) |
| Female Reproductive Tract Infections* | n (%) | 2 (1%) | 0 | 2 (<1%) |
| Vaginitis* | n (%) | 1 (<1%) | 0 | 1 (<1%) |
| Vaginitis Bacterial NOS* | n (%) | 1 (<1%) | 0 | 1 (<1%) |
| Tinea Infections | n (%) | 1 (<1%) | 1 (<1%) | 2 (<1%) |
| Tinea Cruris | n (%) | 0 | 1 (<1%) | 1 (<1%) |
| Tinea Pedis | n (%) | 1 (<1%) | 0 | 1 (<1%) |
| Mycoplasma Infections | n (%) | 1 (<1%) | 0 | 1 (<1%) |
| Pneumonia Mycoplasmal | n (%) | 1 (<1%) | 0 | 1 (<1%) |
| Staphylococcal Infections | n (%) | 1 (<1%) | 0 | 1 (<1%) |
| Staphylococcal Infection NOS | n (%) | 1 (<1%) | 0 | 1 (<1%) |
| Investigations | n (%) | 77 (30%) | 49 (18%) | 126 (24%) |
| Gastrointestinal Histopathology Procedures | n (%) | 25 (10%) | 12 (4%) | 37 (7%) |
| Faecal Occult Blood Positive | n (%) | 23 (9%) | 8 (3%) | 31 (6%) |
| Blood in Stool | n (%) | 4 (2%) | 4 (1%) | 8 (1%) |
| Skeletal and Cardiac Muscle Analyses | n (%) | 19 (7%) | 8 (3%) | 27 (5%) |
| Blood Creatine Phosphokinase Increased | n (%) | 19 (7%) | 8 (3%) | 27 (5%) |
| Blood Creatine Phosphokinase Mb Increased | n (%) | 1 (<1%) | 0 | 1 (<1%) |
| Liver Function Analyses | n (%) | 12 (5%) | 11 (4%) | 23 (4%) |
| Alanine Aminotransferase Increased | n (%) | 8 (3%) | 6 (2%) | 14 (3%) |
| Gamma-Glutamyltransferase Increased | n (%) | 5 (2%) | 3 (1%) | 8 (1%) |
| Aspartate Aminotransferase Increased | n (%) | 4 (2%) | 3 (1%) | 7 (1%) |
| Blood Bilirubin Increased | n (%) | 2 (<1%) | 2 (<1%) | 4 (<1%) |
| Liver Function Tests NOS Abnormal | n (%) | 0 | 2 (<1%) | 2 (<1%) |
| Bilirubin Urine | n (%) | 1 (<1%) | 0 | 1 (<1%) |
| Urine Bilirubin Increased | n (%) | 0 | 1 (<1%) | 1 (<1%) |
| Urobilin Urine Present | n (%) | 1 (<1%) | 0 | 1 (<1%) |
| Cholesterol Analyses | n (%) | 11 (4%) | 8 (3%) | 19 (4%) |
| Blood Cholesterol Increased | n (%) | 11 (4%) | 8 (3%) | 19 (4%) |
| Urinalysis NEC | n (%) | 10 (4%) | 6 (2%) | 16 (3%) |
| Blood Urine Present | n (%) | 6 (2%) | 4 (1%) | 10 (2%) |
| Urine Leukocyte Esterase Positive | n (%) | 3 (1%) | 2 (<1%) | 5 (<1%) |
| Protein Urine Present | n (%) | 2 (<1%) | 1 (<1%) | 3 (<1%) |
| Nitrite Urine Present | n (%) | 1 (<1%) | 1 (<1%) | 2 (<1%) |
| Glucose Urine Present | n (%) | 1 (<1%) | 0 | 1 (<1%) |
| Red Blood Cells Urine | n (%) | 0 | 1 (<1%) | 1 (<1%) |
| Urine Analysis Abnormal NOS | n (%) | 1 (<1%) | 0 | 1 (<1%) |
| White Blood Cells Urine Positive | n (%) | 1 (<1%) | 0 | 1 (<1%) |
| Triglyceride Analyses | n (%) | 6 (2%) | 4 (1%) | 10 (2%) |
| Blood Triglycerides Increased | n (%) | 6 (2%) | 4 (1%) | 10 (2%) |
| Physical Examination Procedures | n (%) | 8 (3%) | 1 (<1%) | 9 (2%) |
| Weight Increased | n (%) | 6 (2%) | 0 | 6 (1%) |
| Weight Decreased | n (%) | 1 (<1%) | 1 (<1%) | 2 (<1%) |
| Prostate Examination Abnormal* | n (%) | 1 (1%) | 0 | 1 (<1%) |
| Metabolism Tests NEC | n (%) | 4 (2%) | 2 (<1%) | 6 (1%) |
| Urine Ketone Body Present | n (%) | 3 (1%) | 2 (<1%) | 5 (<1%) |
| Blood Uric Acid Increased | n (%) | 2 (<1%) | 0 | 2 (<1%) |
| Vascular Tests NEC (Incl Blood Pressure) | n (%) | 3 (1%) | 2 (<1%) | 5 (<1%) |
| Blood Pressure Increased | n (%) | 3 (1%) | 2 (<1%) | 5 (<1%) |
| White Blood Cell Analyses | n (%) | 4 (2%) | 1 (<1%) | 5 (<1%) |
| Neutrophil Count Decreased | n (%) | 3 (1%) | 0 | 3 (<1%) |
| Lymphocyte Count Increased | n (%) | 2 (<1%) | 0 | 2 (<1%) |
| Monocyte Count Decreased | n (%) | 1 (<1%) | 1 (<1%) | 2 (<1%) |
| Monocyte Count Increased | n (%) | 2 (<1%) | 0 | 2 (<1%) |
| White Blood Cell Count Decreased | n (%) | 1 (<1%) | 0 | 1 (<1%) |
| Renal Function Analyses | n (%) | 1 (<1%) | 3 (1%) | 4 (<1%) |
| Blood Creatinine Increased | n (%) | 1 (<1%) | 3 (1%) | 4 (<1%) |
| Tissue Enzyme Analyses NEC | n (%) | 2 (<1%) | 1 (<1%) | 3 (<1%) |
| Blood Alkaline Phosphatase NOS Increased | n (%) | 2 (<1%) | 1 (<1%) | 3 (<1%) |
| Carbohydrate Tolerance Analyses (Incl Diabetes) | n (%) | 2 (<1%) | 0 | 2 (<1%) |
| Blood Glucose Increased | n (%) | 2 (<1%) | 0 | 2 (<1%) |
| ECG Investigations | n (%) | 1 (<1%) | 1 (<1%) | 2 (<1%) |
| Electrocardiogram Qrs Complex Abnormal | n (%) | 1 (<1%) | 0 | 1 (<1%) |
| Electrocardiogram T Wave Inversion | n (%) | 0 | 1 (<1%) | 1 (<1%) |
| Heart Rate and Pulse Investigations | n (%) | 1 (<1%) | 1 (<1%) | 2 (<1%) |
| Heart Rate Increased | n (%) | 1 (<1%) | 1 (<1%) | 2 (<1%) |
| Mineral and Electrolyte Analyses | n (%) | 2 (<1%) | 0 | 2 (<1%) |
| Blood Potassium Increased | n (%) | 1 (<1%) | 0 | 1 (<1%) |
| Blood Sodium Increased | n (%) | 1 (<1%) | 0 | 1 (<1%) |
| Ophthalmic Function Diagnostic Procedures | n (%) | 1 (<1%) | 1 (<1%) | 2 (<1%) |
| Intraocular Pressure Increased | n (%) | 1 (<1%) | 1 (<1%) | 2 (<1%) |
| Vascular Auscultatory Investigations | n (%) | 0 | 2 (<1%) | 2 (<1%) |
| Carotid Bruit | n (%) | 0 | 1 (<1%) | 1 (<1%) |
| Renal Bruit | n (%) | 0 | 1 (<1%) | 1 (<1%) |
| Bacteria Identification and Serology (Excl Mycobacteria) | n (%) | 1 (<1%) | 0 | 1 (<1%) |
| Helicobacter Pylori Antibody Positive | n (%) | 1 (<1%) | 0 | 1 (<1%) |
| Cardiac Auscultatory Investigations | n (%) | 1 (<1%) | 0 | 1 (<1%) |
| Cardiac Murmur NOS | n (%) | 1 (<1%) | 0 | 1 (<1%) |
| Musculoskeletal and Soft Tissue Imaging Procedures | n (%) | 0 | 1 (<1%) | 1 (<1%) |
| Bone Density Decreased | n (%) | 0 | 1 (<1%) | 1 (<1%) |
| Red Blood Cell Analyses | n (%) | 1 (<1%) | 0 | 1 (<1%) |
| Haematocrit Decreased | n (%) | 1 (<1%) | 0 | 1 (<1%) |
| Haemoglobin Decreased | n (%) | 1 (<1%) | 0 | 1 (<1%) |
| Reproductive Organ and Breast Histopathology Procedures | n (%) | 1 (<1%) | 0 | 1 (<1%) |
| Smear Cervix Abnormal* | n (%) | 1 (<1%) | 0 | 1 (<1%) |
| Musculoskeletal and Connective Tissue Disorders | n (%) | 56 (22%) | 31 (11%) | 87 (16%) |
| Musculoskeletal and Connective Tissue Signs and Symptoms NEC | n (%) | 19 (7%) | 14 (5%) | 33 (6%) |
| Back Pain | n (%) | 12 (5%) | 6 (2%) | 18 (3%) |
| Pain in Limb | n (%) | 2 (<1%) | 4 (1%) | 6 (1%) |
| Back Pain Aggravated | n (%) | 2 (<1%) | 1 (<1%) | 3 (<1%) |
| Chest Wall Pain | n (%) | 0 | 2 (<1%) | 2 (<1%) |
| Limb Discomfort NOS | n (%) | 0 | 1 (<1%) | 1 (<1%) |
| Muscle Spasms | n (%) | 1 (<1%) | 0 | 1 (<1%) |
| Musculoskeletal Chest Pain | n (%) | 1 (<1%) | 0 | 1 (<1%) |
| Neck Pain | n (%) | 1 (<1%) | 0 | 1 (<1%) |
| Joint Related Signs and Symptoms | n (%) | 11 (4%) | 3 (1%) | 14 (3%) |
| Arthralgia | n (%) | 10 (4%) | 3 (1%) | 13 (2%) |
| Joint Swelling | n (%) | 1 (<1%) | 0 | 1 (<1%) |
| Muscle Related Signs and Symptoms NEC | n (%) | 9 (4%) | 2 (<1%) | 11 (2%) |
| Muscle Cramps | n (%) | 8 (3%) | 2 (<1%) | 10 (2%) |
| Night Cramps | n (%) | 1 (<1%) | 0 | 1 (<1%) |
| Arthropathies NEC | n (%) | 5 (2%) | 1 (<1%) | 6 (1%) |
| Arthritis NOS | n (%) | 3 (1%) | 0 | 3 (<1%) |
| Monoarthritis | n (%) | 1 (<1%) | 1 (<1%) | 2 (<1%) |
| Arthritis NOS Aggravated | n (%) | 1 (<1%) | 0 | 1 (<1%) |
| Bursal Disorders | n (%) | 3 (1%) | 3 (1%) | 6 (1%) |
| Bursitis | n (%) | 1 (<1%) | 2 (<1%) | 3 (<1%) |
| Bunion | n (%) | 2 (<1%) | 0 | 2 (<1%) |
| Popliteal Bursitis | n (%) | 0 | 1 (<1%) | 1 (<1%) |
| Intervertebral Disc Disorders NEC | n (%) | 5 (2%) | 0 | 5 (<1%) |
| Intervertebral Disc Herniation | n (%) | 4 (2%) | 0 | 4 (<1%) |
| Intervertebral Disc Degeneration NOS | n (%) | 2 (<1%) | 0 | 2 (<1%) |
| Intervertebral Disc Disorder NOS | n (%) | 1 (<1%) | 0 | 1 (<1%) |
| Connective Tissue Disorders (Excl Le) | n (%) | 2 (<1%) | 2 (<1%) | 4 (<1%) |
| Plantar Fasciitis | n (%) | 2 (<1%) | 2 (<1%) | 4 (<1%) |
| Osteoarthropathies | n (%) | 4 (2%) | 0 | 4 (<1%) |
| Osteoarthritis Aggravated | n (%) | 3 (1%) | 0 | 3 (<1%) |
| Localised Osteoarthritis | n (%) | 1 (<1%) | 0 | 1 (<1%) |
| Bone Disorders NEC | n (%) | 1 (<1%) | 2 (<1%) | 3 (<1%) |
| Bone Disorder NOS | n (%) | 1 (<1%) | 0 | 1 (<1%) |
| Bone Spur | n (%) | 0 | 1 (<1%) | 1 (<1%) |
| Osteopenia | n (%) | 0 | 1 (<1%) | 1 (<1%) |
| Metabolic Bone Disorders | n (%) | 2 (<1%) | 1 (<1%) | 3 (<1%) |
| Osteoporosis NOS | n (%) | 2 (<1%) | 1 (<1%) | 3 (<1%) |
| Muscle Pains | n (%) | 2 (<1%) | 1 (<1%) | 3 (<1%) |
| Myalgia | n (%) | 1 (<1%) | 1 (<1%) | 2 (<1%) |
| Fibromyalgia | n (%) | 1 (<1%) | 0 | 1 (<1%) |
| Tendon Disorders | n (%) | 0 | 3 (1%) | 3 (<1%) |
| Tendonitis | n (%) | 0 | 2 (<1%) | 2 (<1%) |
| Trigger Finger | n (%) | 0 | 1 (<1%) | 1 (<1%) |
| Joint Related Disorders NEC | n (%) | 1 (<1%) | 1 (<1%) | 2 (<1%) |
| Rotator Cuff Syndrome | n (%) | 0 | 1 (<1%) | 1 (<1%) |
| Temporomandibular Joint Disorder NOS | n (%) | 1 (<1%) | 0 | 1 (<1%) |
| Rheumatoid Arthropathies | n (%) | 0 | 2 (<1%) | 2 (<1%) |
| Rheumatoid Arthritis | n (%) | 0 | 1 (<1%) | 1 (<1%) |
| Rheumatoid Arthritis Aggravated | n (%) | 0 | 1 (<1%) | 1 (<1%) |
| Soft Tissue Disorders NEC | n (%) | 2 (<1%) | 0 | 2 (<1%) |
| Groin Pain | n (%) | 1 (<1%) | 0 | 1 (<1%) |
| Peripheral Swelling | n (%) | 1 (<1%) | 0 | 1 (<1%) |
| Cartilage Disorders | n (%) | 1 (<1%) | 0 | 1 (<1%) |
| Cartilage Disorder NOS | n (%) | 1 (<1%) | 0 | 1 (<1%) |
| Muscle Infections and Inflammations | n (%) | 0 | 1 (<1%) | 1 (<1%) |
| Myositis | n (%) | 0 | 1 (<1%) | 1 (<1%) |
| Muscle Tone Abnormalities | n (%) | 1 (<1%) | 0 | 1 (<1%) |
| Cervical Spasm | n (%) | 1 (<1%) | 0 | 1 (<1%) |
| Spondyloarthropathies | n (%) | 1 (<1%) | 0 | 1 (<1%) |
| Spondylosis | n (%) | 1 (<1%) | 0 | 1 (<1%) |
| Nervous System Disorders | n (%) | 41 (16%) | 23 (8%) | 64 (12%) |
| Headaches NEC | n (%) | 25 (10%) | 14 (5%) | 39 (7%) |
| Headache NOS | n (%) | 19 (7%) | 12 (4%) | 31 (6%) |
| Headache NOS Aggravated | n (%) | 3 (1%) | 2 (<1%) | 5 (<1%) |
| Sinus Headache | n (%) | 3 (1%) | 0 | 3 (<1%) |
| Tension Headaches | n (%) | 1 (<1%) | 0 | 1 (<1%) |
| Neurological Signs and Symptoms NEC | n (%) | 4 (2%) | 2 (<1%) | 6 (1%) |
| Dizziness | n (%) | 4 (2%) | 2 (<1%) | 6 (1%) |
| Migraine Headaches | n (%) | 5 (2%) | 0 | 5 (<1%) |
| Migraine NOS | n (%) | 5 (2%) | 0 | 5 (<1%) |
| Disturbances in Consciousness NEC | n (%) | 3 (1%) | 1 (<1%) | 4 (<1%) |
| Somnolence | n (%) | 1 (<1%) | 1 (<1%) | 2 (<1%) |
| Loss of Consciousness | n (%) | 1 (<1%) | 0 | 1 (<1%) |
| Vasovagal Attack | n (%) | 1 (<1%) | 0 | 1 (<1%) |
| Mononeuropathies | n (%) | 2 (<1%) | 1 (<1%) | 3 (<1%) |
| Carpal Tunnel Syndrome | n (%) | 2 (<1%) | 1 (<1%) | 3 (<1%) |
| Paraesthesias and Dysaesthesias | n (%) | 1 (<1%) | 1 (<1%) | 2 (<1%) |
| Hypoaesthesia | n (%) | 1 (<1%) | 1 (<1%) | 2 (<1%) |
| Sleep Apnoeas | n (%) | 0 | 2 (<1%) | 2 (<1%) |
| Sleep Apnoea Syndrome | n (%) | 0 | 2 (<1%) | 2 (<1%) |
| Tremor (Excl Congenital) | n (%) | 1 (<1%) | 1 (<1%) | 2 (<1%) |
| Tremor | n (%) | 1 (<1%) | 1 (<1%) | 2 (<1%) |
| Abnormal Reflexes | n (%) | 1 (<1%) | 0 | 1 (<1%) |
| Hyperreflexia | n (%) | 1 (<1%) | 0 | 1 (<1%) |
| Dyskinesias and Movement Disorders NEC | n (%) | 0 | 1 (<1%) | 1 (<1%) |
| Movement Disorder NOS | n (%) | 0 | 1 (<1%) | 1 (<1%) |
| Memory Loss (Excl Dementia) | n (%) | 1 (<1%) | 0 | 1 (<1%) |
| Transient Global Amnesia | n (%) | 1 (<1%) | 0 | 1 (<1%) |
| Neuromuscular Junction Dysfunction | n (%) | 0 | 1 (<1%) | 1 (<1%) |
| Myasthenia Gravis | n (%) | 0 | 1 (<1%) | 1 (<1%) |
| Parkinson's Disease and Parkinsonism | n (%) | 1 (<1%) | 0 | 1 (<1%) |
| Parkinson's Disease NOS | n (%) | 1 (<1%) | 0 | 1 (<1%) |
| Seizures and Seizure Disorders NEC | n (%) | 1 (<1%) | 0 | 1 (<1%) |
| Status Epilepticus | n (%) | 1 (<1%) | 0 | 1 (<1%) |
| Speech and Language Abnormalities | n (%) | 0 | 1 (<1%) | 1 (<1%) |
| Dysarthria | n (%) | 0 | 1 (<1%) | 1 (<1%) |
| Spinal Cord and Nerve Root Disorders NEC | n (%) | 0 | 1 (<1%) | 1 (<1%) |
| Myeloradiculopathy | n (%) | 0 | 1 (<1%) | 1 (<1%) |
| Spondylitic Myelopathy | n (%) | 0 | 1 (<1%) | 1 (<1%) |
| Injury, Poisoning and Procedural Complications | n (%) | 26 (10%) | 21 (8%) | 47 (9%) |
| Non-Site Specific Injuries NEC | n (%) | 5 (2%) | 6 (2%) | 11 (2%) |
| Laceration | n (%) | 3 (1%) | 3 (1%) | 6 (1%) |
| Arthropod Bite | n (%) | 1 (<1%) | 1 (<1%) | 2 (<1%) |
| Animal Bite | n (%) | 0 | 1 (<1%) | 1 (<1%) |
| Arthropod Sting | n (%) | 0 | 1 (<1%) | 1 (<1%) |
| Road Traffic Accident | n (%) | 1 (<1%) | 0 | 1 (<1%) |
| Muscle, Tendon and Ligament Injuries | n (%) | 6 (2%) | 4 (1%) | 10 (2%) |
| Muscle Strain | n (%) | 5 (2%) | 3 (1%) | 8 (1%) |
| Ligament Sprain | n (%) | 1 (<1%) | 1 (<1%) | 2 (<1%) |
| Non-Site Specific Procedural Complications | n (%) | 3 (1%) | 5 (2%) | 8 (1%) |
| Post Procedural Pain | n (%) | 2 (<1%) | 4 (1%) | 6 (1%) |
| Post Procedural Discomfort | n (%) | 1 (<1%) | 1 (<1%) | 2 (<1%) |
| Site Specific Injuries NEC | n (%) | 6 (2%) | 0 | 6 (1%) |
| Back Injury NOS | n (%) | 3 (1%) | 0 | 3 (<1%) |
| Limb Injury NOS | n (%) | 2 (<1%) | 0 | 2 (<1%) |
| Bone Injury | n (%) | 1 (<1%) | 0 | 1 (<1%) |
| Limb Injuries NEC (Incl Traumatic Amputation) | n (%) | 1 (<1%) | 3 (1%) | 4 (<1%) |
| Joint Sprain | n (%) | 1 (<1%) | 3 (1%) | 4 (<1%) |
| Lower Limb Fractures and Dislocations | n (%) | 1 (<1%) | 2 (<1%) | 3 (<1%) |
| Foot Fracture | n (%) | 1 (<1%) | 1 (<1%) | 2 (<1%) |
| Lower Limb Fracture NOS | n (%) | 0 | 1 (<1%) | 1 (<1%) |
| Eye Injuries NEC | n (%) | 1 (<1%) | 0 | 1 (<1%) |
| Periorbital Haematoma | n (%) | 1 (<1%) | 0 | 1 (<1%) |
| Fractures and Dislocations NEC | n (%) | 1 (<1%) | 0 | 1 (<1%) |
| Stress Fracture | n (%) | 1 (<1%) | 0 | 1 (<1%) |
| Heat Injuries (Excl Thermal Burns) | n (%) | 1 (<1%) | 0 | 1 (<1%) |
| Heat Exhaustion | n (%) | 1 (<1%) | 0 | 1 (<1%) |
| Overdoses | n (%) | 1 (<1%) | 0 | 1 (<1%) |
| Non-Accidental Overdose | n (%) | 1 (<1%) | 0 | 1 (<1%) |
| Thoracic Cage Fractures and Dislocations | n (%) | 1 (<1%) | 0 | 1 (<1%) |
| Rib Fracture | n (%) | 1 (<1%) | 0 | 1 (<1%) |
| Upper Limb Fractures and Dislocations | n (%) | 0 | 1 (<1%) | 1 (<1%) |
| Hand Fracture | n (%) | 0 | 1 (<1%) | 1 (<1%) |
| Respiratory, Thoracic and Mediastinal Disorders | n (%) | 28 (11%) | 18 (6%) | 46 (9%) |
| Coughing and Associated Symptoms | n (%) | 6 (2%) | 6 (2%) | 12 (2%) |
| Cough | n (%) | 5 (2%) | 6 (2%) | 11 (2%) |
| Productive Cough | n (%) | 1 (<1%) | 0 | 1 (<1%) |
| Paranasal Sinus Disorders (Excl Infections and Neoplasms) | n (%) | 6 (2%) | 3 (1%) | 9 (2%) |
| Sinus Congestion | n (%) | 5 (2%) | 3 (1%) | 8 (1%) |
| Paranasal Sinus Hypersecretion | n (%) | 1 (<1%) | 0 | 1 (<1%) |
| Nasal Congestion and Inflammations | n (%) | 7 (3%) | 1 (<1%) | 8 (1%) |
| Rhinitis Allergic NOS | n (%) | 3 (1%) | 1 (<1%) | 4 (<1%) |
| Rhinitis NOS | n (%) | 3 (1%) | 0 | 3 (<1%) |
| Rhinitis Seasonal | n (%) | 1 (<1%) | 0 | 1 (<1%) |
| Upper Respiratory Tract Signs and Symptoms | n (%) | 3 (1%) | 4 (1%) | 7 (1%) |
| Pharyngitis | n (%) | 3 (1%) | 3 (1%) | 6 (1%) |
| Postnasal Drip | n (%) | 0 | 1 (<1%) | 1 (<1%) |
| Bronchospasm and Obstruction | n (%) | 3 (1%) | 1 (<1%) | 4 (<1%) |
| Asthma NOS | n (%) | 1 (<1%) | 1 (<1%) | 2 (<1%) |
| Asthma Aggravated | n (%) | 1 (<1%) | 0 | 1 (<1%) |
| Wheezing | n (%) | 1 (<1%) | 0 | 1 (<1%) |
| Breathing Abnormalities | n (%) | 2 (<1%) | 1 (<1%) | 3 (<1%) |
| Dyspnoea NOS | n (%) | 2 (<1%) | 1 (<1%) | 3 (<1%) |
| Laryngeal and Adjacent Sites Disorders NEC (Excl Infections and | n (%) | 2 (<1%) | 1 (<1%) | 3 (<1%) |
| Neoplasms) | | | | |
| Laryngitis NOS | n (%) | 2 (<1%) | 1 (<1%) | 3 (<1%) |
| Lower Respiratory Tract Signs and Symptoms | n (%) | 1 (<1%) | 1 (<1%) | 2 (<1%) |
| Abnormal Chest Sounds NOS | n (%) | 0 | 1 (<1%) | 1 (<1%) |
| Breath Sounds Decreased | n (%) | 1 (<1%) | 0 | 1 (<1%) |
| Respiratory Tract Disorders NEC | n (%) | 1 (<1%) | 1 (<1%) | 2 (<1%) |
| Respiratory Tract Congestion | n (%) | 1 (<1%) | 1 (<1%) | 2 (<1%) |
| General Disorders and Administration Site Conditions | n (%) | 23 (9%) | 19 (7%) | 42 (8%) |
| Asthenic Conditions | n (%) | 8 (3%) | 7 (3%) | 15 (3%) |
| Fatigue | n (%) | 7 (3%) | 6 (2%) | 13 (2%) |
| Fatigue Aggravated | n (%) | 1 (<1%) | 0 | 1 (<1%) |
| Lethargy | n (%) | 0 | 1 (<1%) | 1 (<1%) |
| Pain and Discomfort NEC | n (%) | 5 (2%) | 7 (3%) | 12 (2%) |
| Chest Pain | n (%) | 3 (1%) | 4 (1%) | 7 (1%) |
| Pain NOS | n (%) | 2 (<1%) | 1 (<1%) | 3 (<1%) |
| Chest Discomfort | n (%) | 0 | 1 (<1%) | 1 (<1%) |
| Tenderness NOS | n (%) | 0 | 1 (<1%) | 1 (<1%) |
| General Signs and Symptoms NEC | n (%) | 6 (2%) | 3 (1%) | 9 (2%) |
| Fall | n (%) | 2 (<1%) | 2 (<1%) | 4 (<1%) |
| Influenza Like Illness | n (%) | 3 (1%) | 1 (<1%) | 4 (1%) |
| Chest Tightness | n (%) | 1 (<1%) | 0 | 1 (<1%) |
| Oedema NEC | n (%) | 4 (2%) | 3 (1%) | 7 (1%) |
| Oedema Peripheral | n (%) | 4 (2%) | 2 (<1%) | 6 (1%) |
| Oedema NOS | n (%) | 0 | 1 (<1%) | 1 (<1%) |
| Febrile Disorders | n (%) | 2 (<1%) | 1 (<1%) | 3 (<1%) |
| Pyrexia | n (%) | 2 (<1%) | 1 (<1%) | 3 (<1%) |
| Hernias NEC | n (%) | 0 | 1 (<1%) | 1 (<1%) |
| Hernia NOS | n (%) | 0 | 1 (<1%) | 1 (<1%) |
| Skin and Subcutaneous Tissue Disorders | n (%) | 14 (5%) | 25 (9%) | 39 (7%) |
| Rashes, Eruptions and Exanthems NEC | n (%) | 4 (2%) | 4 (1%) | 8 (1%) |
| Rash NOS | n (%) | 4 (2%) | 2 (<1%) | 6 (1%) |
| Rash Macular | n (%) | 0 | 2 (<1%) | 2 (<1%) |
| Dermatitis and Eczema | n (%) | 2 (<1%) | 4 (1%) | 6 (1%) |
| Dermatitis Contact | n (%) | 1 (<1%) | 1 (<1%) | 2 (<1%) |
| Eczema | n (%) | 1 (<1%) | 1 (<1%) | 2 (<1%) |
| Dermatitis Allergic | n (%) | 0 | 1 (<1%) | 1 (<1%) |
| Dermatitis NOS | n (%) | 0 | 1 (<1%) | 1 (<1%) |
| Dermal and Epidermal Conditions NEC | n (%) | 1 (<1%) | 4 (1%) | 5 (<1%) |
| Skin Lesion NOS | n (%) | 1 (<1%) | 2 (<1%) | 3 (<1%) |
| Skin Disorder NOS | n (%) | 0 | 1 (<1%) | 1 (<1%) |
| Swelling Face | n (%) | 0 | 1 (<1%) | 1 (<1%) |
| Pruritus NEC | n (%) | 3 (1%) | 1 (<1%) | 4 (<1%) |
| Pruritus NOS | n (%) | 3 (1%) | 1 (<1%) | 4 (<1%) |
| Apocrine and Eccrine Gland Disorders | n (%) | 0 | 3 (1%) | 3 (<1%) |
| Sweating Increased | n (%) | 0 | 2 (<1%) | 2 (<1%) |
| Dyshidrosis | n (%) | 0 | 1 (<1%) | 1 (<1%) |
| Skin Injuries and Mechanical Dermatoses | n (%) | 1 (<1%) | 2 (<1%) | 3 (<1%) |
| Contusion | n (%) | 1 (<1%) | 2 (<1%) | 3 (<1%) |
| Urticarias | n (%) | 1 (<1%) | 2 (<1%) | 3 (<1%) |
| Urticaria NOS | n (%) | 1 (<1%) | 2 (<1%) | 3 (<1%) |
| Dermographism | n (%) | 0 | 1 (<1%) | 1 (<1%) |
| Acnes | n (%) | 1 (<1%) | 0 | 1 (<1%) |
| Acne NOS | n (%) | 1 (<1%) | 0 | 1 (<1%) |
| Alopecias | n (%) | 0 | 1 (<1%) | 1 (<1%) |
| Alopecia | n (%) | 0 | 1 (<1%) | 1 (<1%) |
| Dermatitis Ascribed to Specific Agent | n (%) | 0 | 1 (<1%) | 1 (<1%) |
| Dermatitis Medicamentosa | n (%) | 0 | 1 (<1%) | 1 (<1%) |
| Erythemas | n (%) | 1 (<1%) | 0 | 1 (<1%) |
| Erythema | n (%) | 1 (<1%) | 0 | 1 (<1%) |
| Hyperkeratoses | n (%) | 0 | 1 (<1%) | 1 (<1%) |
| Hyperkeratosis | n (%) | 0 | 1 (<1%) | 1 (<1%) |
| Nail and Nail Bed Conditions (Excl Infections and Infestations) | n (%) | 0 | 1 (<1%) | 1 (<1%) |
| Ingrowing Nail | n (%) | 0 | 1 (<1%) | 1 (<1%) |
| Papulosquamous Conditions | n (%) | 0 | 1 (<1%) | 1 (<1%) |
| Psoriasis | n (%) | 0 | 1 (<1%) | 1 (<1%) |
| Rosaceas | n (%) | 1 (<1%) | 0 | 1 (<1%) |
| Rosacea | n (%) | 1 (<1%) | 0 | 1 (<1%) |
| Skin Neoplasms Benign | n (%) | 0 | 1 (<1%) | 1 (<1%) |
| Acrochordons | n (%) | 0 | 1 (<1%) | 1 (<1%) |
| Skin and Subcutaneous Tissue Ulcerations | n (%) | 0 | 1 (<1%) | 1 (<1%) |
| Skin Ulcer | n (%) | 0 | 1 (<1%) | 1 (<1%) |
| Psychiatric Disorders | n (%) | 15 (6%) | 17 (6%) | 32 (6%) |
| Depressive Disorders | n (%) | 7 (3%) | 6 (2%) | 13 (2%) |
| Depression | n (%) | 4 (2%) | 5 (2%) | 9 (2%) |
| Depression Aggravated | n (%) | 3 (1%) | 1 (<1%) | 4 (<1%) |
| Anxiety Symptoms | n (%) | 5 (2%) | 7 (3%) | 12 (2%) |
| Anxiety | n (%) | 3 (1%) | 5 (2%) | 8 (1%) |
| Anxiety Aggravated | n (%) | 2 (<1%) | 1 (<1%) | 3 (<1%) |
| Stress Symptoms | n (%) | 0 | 1 (<1%) | 1 (<1%) |
| Disturbances in Initiating and Maintaining Sleep | n (%) | 6 (2%) | 0 | 6 (1%) |
| Insomnia | n (%) | 5 (2%) | 0 | 5 (<1%) |
| Insomnia Exacerbated | n (%) | 1 (<1%) | 0 | 1 (<1%) |
| Behaviour and Socialisation Disturbances | n (%) | 0 | 1 (<1%) | 1 (<1%) |
| Irritability | n (%) | 0 | 1 (<1%) | 1 (<1%) |
| Mental Disorders NEC | n (%) | 0 | 1 (<1%) | 1 (<1%) |
| Mental Status Changes | n (%) | 0 | 1 (<1%) | 1 (<1%) |
| Panic Symptoms | n (%) | 0 | 1 (<1%) | 1 (<1%) |
| Panic Attack | n (%) | 0 | 1 (<1%) | 1 (<1%) |
| Perception Disturbances | n (%) | 0 | 1 (<1%) | 1 (<1%) |
| Hallucination NOS | n (%) | 0 | 1 (<1%) | 1 (<1%) |
| Hallucination, Auditory | n (%) | 0 | 1 (<1%) | 1 (<1%) |
| Psychotic Disorder NEC | n (%) | 0 | 1 (<1%) | 1 (<1%) |
| Psychotic Disorder NOS | n (%) | 0 | 1 (<1%) | 1 (<1%) |
| Sexual Desire Disorders | n (%) | 0 | 1 (<1%) | 1 (<1%) |
| Libido Decreased | n (%) | 0 | 1 (<1%) | 1 (<1%) |
| Suicidal and Self-Injurious Behaviour | n (%) | 0 | 1 (<1%) | 1 (<1%) |
| Suicidal Ideation | n (%) | 0 | 1 (<1%) | 1 (<1%) |
| Reproductive System and Breast Disorders | n (%) | 12 (5%) | 12 (4%) | 24 (4%) |
| Breast Disorders NEC* | n (%) | 1 (<1%) | 4 (2%) | 5 (1%) |
| Breast Mass NOS* | n (%) | 1 (<1%) | 2 (1%) | 3 (<1%) |
| Breast Calcifications* | n (%) | 0 | 1 (<1%) | 1 (<1%) |
| Breast Disorder NOS* | n (%) | 0 | 1 (<1%) | 1 (<1%) |
| Menstruation with Increased Bleeding* | n (%) | 2 (1%) | 1 (<1%) | 3 (<1%) |
| Menorrhagia* | n (%) | 1 (<1%) | 1 (<1%) | 2 (<1%) |
| Intermenstrual Bleeding* | n (%) | 1 (<1%) | 0 | 1 (<1%) |
| Prostate and Seminal Vesicles Infections and Inflammations* | n (%) | 2 (2%) | 1 (1%) | 3 (2%) |
| Prostatitis* | n (%) | 2 (2%) | 1 (1%) | 3 (2%) |
| Reproductive Tract Signs and Symptoms NEC | n (%) | 1 (<1%) | 2 (<1%) | 3 (<1%) |
| Pelvic Pain NOS | n (%) | 0 | 2 (<1%) | 2 (<1%) |
| Premenstrual Syndrome* | n (%) | 1 (<1%) | 0 | 1 (<1%) |
| Vulvovaginal Signs and Symptoms* | n (%) | 1 (<1%) | 2 (1%) | 3 (<1%) |
| Vulvovaginal Dryness* | n (%) | 0 | 2 (1%) | 2 (<1%) |
| Vaginal Lesion* | n (%) | 1 (<1%) | 0 | 1 (<1%) |
| Menstruation and Uterine Bleeding NEC* | n (%) | 1 (<1%) | 1 (<1%) | 2 (<1%) |
| Dysfunctional Uterine Bleeding* | n (%) | 1 (<1%) | 0 | 1 (<1%) |
| Menstruation Irregular* | n (%) | 0 | 1 (<1%) | 1 (<1%) |
| Uterine Disorders NEC* | n (%) | 2 (1%) | 0 | 2 (<1%) |
| Endometriosis* | n (%) | 2 (1%) | 0 | 2 (<1%) |
| Adenomyosis* | n (%) | 1 (<1%) | 0 | 1 (<1%) |
| Uterine Neoplasms* | n (%) | 2 (1%) | 0 | 2 (<1%) |
| Uterine Polyp NOS* | n (%) | 2 (1%) | 0 | 2 (<1%) |
| Menstruation with Decreased Bleeding* | n (%) | 1 (<1%) | 0 | 1 (<1%) |
| Amenorrhoea NOS* | n (%) | 1 (<1%) | 0 | 1 (<1%) |
| Penile Disorders NEC (Excl Erection and Ejaculation)* | n (%) | 0 | 1 (1%) | 1 (<1%) |
| Penile Haemorrhage* | n (%) | 0 | 1 (1%) | 1 (<1%) |
| Vulvovaginal Cysts and Neoplasms* | n (%) | 1 (<1%) | 0 | 1 (<1%) |
| Vaginal Cyst* | n (%) | 1 (<1%) | 0 | 1 (<1%) |
| Metabolism and Nutrition Disorders | n (%) | 11 (4%) | 10 (4%) | 21 (4%) |
| Elevated Cholesterol | n (%) | 3 (1%) | 3 (1%) | 6 (1%) |
| Hypercholesterolaemia | n (%) | 3 (1%) | 3 (1%) | 6 (1%) |
| Hyperglycaemic Conditions NEC | n (%) | 0 | 4 (1%) | 4 (<1%) |
| Hyperglycaemia NOS | n (%) | 0 | 3 (1%) | 3 (<1%) |
| Glucose Tolerance Impaired | n (%) | 0 | 1 (<1%) | 1 (<1%) |
| Diabetes Mellitus (Incl Subtypes) | n (%) | 2 (<1%) | 1 (<1%) | 3 (<1%) |
| Diabetes Mellitus Aggravated | n (%) | 1 (<1%) | 1 (<1%) | 2 (<1%) |
| Diabetes Mellitus Non-Insulin-Dependent | n (%) | 1 (<1%) | 0 | 1 (<1%) |
| Appetite Disorders | n (%) | 1 (<1%) | 1 (<1%) | 2 (<1%) |
| Anorexia | n (%) | 1 (<1%) | 1 (<1%) | 2 (<1%) |
| Purine Metabolism Disorders NEC | n (%) | 2 (<1%) | 0 | 2 (<1%) |
| Gout | n (%) | 2 (<1%) | 0 | 2 (<1%) |
| Total Fluid Volume Decreased | n (%) | 1 (<1%) | 1 (<1%) | 2 (<1%) |
| Dehydration | n (%) | 1 (<1%) | 1 (<1%) | 2 (<1%) |
| Fluid Intake Increased | n (%) | 0 | 1 (<1%) | 1 (<1%) |
| Polydipsia | n (%) | 0 | 1 (<1%) | 1 (<1%) |
| Hyperlipidaemias NEC | n (%) | 1 (<1%) | 0 | 1 (<1%) |
| Hyperlipidaemia NOS | n (%) | 1 (<1%) | 0 | 1 (<1%) |
| Lipid Metabolism and Deposit Disorders NEC | n (%) | 1 (<1%) | 0 | 1 (<1%) |
| Lipid Metabolism Disorder NOS | n (%) | 1 (<1%) | 0 | 1 (<1%) |
| Sugar Intolerance (Excl Glucose Intolerance) | n (%) | 0 | 1 (<1%) | 1 (<1%) |
| Lactose Intolerance | n (%) | 0 | 1 (<1%) | 1 (<1%) |
| Vascular Disorders | n (%) | 13 (5%) | 8 (3%) | 21 (4%) |
| Vascular Hypertensive Disorders NEC | n (%) | 8 (3%) | 7 (3%) | 15 (3%) |
| Hypertension NOS | n (%) | 7 (3%) | 6 (2%) | 13 (2%) |
| Hypertension Aggravated | n (%) | 1 (<1%) | 1 (<1%) | 2 (<1%) |
| Peripheral Vascular Disorders NEC | n (%) | 2 (<1%) | 0 | 2 (<1%) |
| Flushing | n (%) | 1 (<1%) | 0 | 1 (<1%) |
| Hot Flushes NOS | n (%) | 1 (<1%) | 0 | 1 (<1%) |
| Varicose Veins Non-Site Specific | n (%) | 2 (<1%) | 0 | 2 (<1%) |
| Varicose Vein Ruptured | n (%) | 2 (<1%) | 0 | 2 (<1%) |
| Bruising, Ecchymosis and Purpura | n (%) | 1 (<1%) | 0 | 1 (<1%) |
| Haematoma NOS | n (%) | 1 (<1%) | 0 | 1 (<1%) |
| Peripheral Embolism and Thrombosis | n (%) | 0 | 1 (<1%) | 1 (<1%) |
| Peripheral Embolism NOS | n (%) | 0 | 1 (<1%) | 1 (<1%) |
| Thrombophlebitis Superficial | n (%) | 0 | 1 (<1%) | 1 (<1%) |
| Vascular Hypotensive Disorders | n (%) | 0 | 1 (<1%) | 1 (<1%) |
| Hypotension NOS | n (%) | 0 | 1 (<1%) | 1 (<1%) |
| Renal and Urinary Disorders | n (%) | 13 (5%) | 4 (1%) | 17 (3%) |
| Bladder and Urethral Symptoms | n (%) | 6 (2%) | 1 (<1%) | 7 (1%) |
| Urinary Frequency | n (%) | 2 (<1%) | 1 (<1%) | 3 (<1%) |
| Dysuria | n (%) | 1 (<1%) | 0 | 1 (<1%) |
| Stress Incontinence | n (%) | 1 (<1%) | 0 | 1 (<1%) |
| Urinary Incontinence | n (%) | 1 (<1%) | 0 | 1 (<1%) |
| Urinary Retention | n (%) | 1 (<1%) | 0 | 1 (<1%) |
| Urinary Abnormalities | n (%) | 5 (2%) | 1 (<1%) | 6 (1%) |
| Haematuria | n (%) | 4 (2%) | 0 | 4 (<1%) |
| Glycosuria | n (%) | 0 | 1 (<1%) | 1 (<1%) |
| Proteinuria | n (%) | 1 (<1%) | 0 | 1 (<1%) |
| Urine Abnormal NOS | n (%) | 1 (<1%) | 0 | 1 (<1%) |
| Renal Lithiasis | n (%) | 3 (1%) | 0 | 3 (<1%) |
| Calculus Renal NOS | n (%) | 3 (1%) | 0 | 3 (<1%) |
| Bladder Disorders NEC | n (%) | 0 | 1 (<1%) | 1 (<1%) |
| Bladder Prolapse | n (%) | 0 | 1 (<1%) | 1 (<1%) |
| Bladder Infections and Inflammations | n (%) | 0 | 1 (<1%) | 1 (<1%) |
| Cystitis Interstitial | n (%) | 0 | 1 (<1%) | 1 (<1%) |
| Urinary Tract Lithiasis (Excl Renal) | n (%) | 1 (<1%) | 0 | 1 (<1%) |
| Calculus Ureteric | n (%) | 1 (<1%) | 0 | 1 (<1%) |
| Urinary Tract Signs and Symptoms NEC | n (%) | 0 | 1 (<1%) | 1 (<1%) |
| Polyuria | n (%) | 0 | 1 (<1%) | 1 (<1%) |
| Cardiac Disorders | n (%) | 10 (4%) | 6 (2%) | 16 (3%) |
| Cardiac Signs and Symptoms NEC | n (%) | 2 (<1%) | 2 (<1%) | 4 (<1%) |
| Palpitations | n (%) | 2 (<1%) | 2 (<1%) | 4 (<1%) |
| Ischaemic Coronary Artery Disorders | n (%) | 4 (2%) | 0 | 4 (<1%) |
| Angina Pectoris | n (%) | 3 (1%) | 0 | 3 (<1%) |
| Myocardial Infarction | n (%) | 1 (<1%) | 0 | 1 (<1%) |
| Rate and Rhythm Disorders NEC | n (%) | 2 (<1%) | 2 (<1%) | 4 (<1%) |
| Arrhythmia NOS | n (%) | 1 (<1%) | 0 | 1 (<1%) |
| Bradyarrhythmia | n (%) | 0 | 1 (<1%) | 1 (<1%) |
| Cardiac Flutter | n (%) | 0 | 1 (<1%) | 1 (<1%) |
| Tachycardia NOS | n (%) | 1 (<1%) | 0 | 1 (<1%) |
| Supraventricular Arrhythmias | n (%) | 1 (<1%) | 3 (1%) | 4 (<1%) |
| Atrial Fibrillation | n (%) | 1 (<1%) | 0 | 1 (<1%) |
| Sick Sinus Syndrome | n (%) | 0 | 1 (<1%) | 1 (<1%) |
| Sinus Arrhythmia | n (%) | 0 | 1 (<1%) | 1 (<1%) |
| Supraventricular Tachycardia | n (%) | 0 | 1 (<1%) | 1 (<1%) |
| Cardiac Conduction Disorders | n (%) | 2 (<1%) | 0 | 2 (<1%) |
| Atrioventricular Block First Degree | n (%) | 1 (<1%) | 0 | 1 (<1%) |
| Bundle Branch Block Right | n (%) | 1 (<1%) | 0 | 1 (<1%) |
| Ventricular Arrhythmias and Cardiac Arrest | n (%) | 1 (<1%) | 1 (<1%) | 2 (<1%) |
| Cardiac Fibrillation NOS | n (%) | 0 | 1 (<1%) | 1 (<1%) |
| Ventricular Extrasystoles | n (%) | 1 (<1%) | 0 | 1 (<1%) |
| Neoplasms Benign, Malignant and Unspecified (Incl Cysts and Polyps) | n (%) | 9 (4%) | 6 (2%) | 15 (3%) |
| Skin Neoplasms Malignant and Unspecified (Excl Melanoma) | n (%) | 2 (<1%) | 4 (1%) | 6 (1%) |
| Basal Cell Carcinoma | n (%) | 2 (<1%) | 4 (1%) | 6 (1%) |
| Soft Tissue Neoplasms Benign NEC | n (%) | 3 (1%) | 0 | 3 (<1%) |
| Lipoma NOS | n (%) | 2 (<1%) | 0 | 2 (<1%) |
| Leiomyoma NOS | n (%) | 1 (<1%) | 0 | 1 (<1%) |
| Breast and Nipple Neoplasms Malignant* | n (%) | 0 | 2 (1%) | 2 (<1%) |
| Breast Cancer NOS* | n (%) | 0 | 2 (1%) | 2 (<1%) |
| Neoplasms Benign Site Unspecified NEC | n (%) | 1 (<1%) | 1 (<1%) | 2 (<1%) |
| Cyst NOS | n (%) | 1 (<1%) | 0 | 1 (<1%) |
| Fibroma NOS | n (%) | 0 | 1 (<1%) | 1 (<1%) |
| Bone Neoplasms Malignant (Excl Sarcomas) | n (%) | 1 (<1%) | 0 | 1 (<1%) |
| Bone Neoplasm Malignant | n (%) | 1 (<1%) | 0 | 1 (<1%) |
| Ovarian Neoplasms Benign* | n (%) | 0 | 1 (<1%) | 1 (<1%) |
| Benign Ovarian Tumour* | n (%) | 0 | 1 (<1%) | 1 (<1%) |
| Ovarian Cystadenofibroma* | n (%) | 0 | 1 (<1%) | 1 (<1%) |
| Ovarian Neoplasms Malignant (Excl Germ Cell)* | n (%) | 1 (<1%) | 0 | 1 (<1%) |
| Ovarian Cancer NOS* | n (%) | 1 (<1%) | 0 | 1 (<1%) |
| Uterine Neoplasms Benign* | n (%) | 1 (<1%) | 0 | 1 (<1%) |
| Uterine Fibroids* | n (%) | 1 (<1%) | 0 | 1 (<1%) |
| Eye Disorders | n (%) | 8 (3%) | 5 (2%) | 13 (2%) |
| Cataracts (Excl Congenital) | n (%) | 1 (<1%) | 1 (<1%) | 2 (<1%) |
| Cataract Unilateral | n (%) | 1 (<1%) | 0 | 1 (<1%) |
| Posterior Capsule Opacification | n (%) | 0 | 1 (<1%) | 1 (<1%) |
| Conjunctival Infections, Irritations and Inflammations | n (%) | 2 (<1%) | 0 | 2 (<1%) |
| Conjunctivitis | n (%) | 1 (<1%) | 0 | 1 (<1%) |
| Conjunctivitis Allergic | n (%) | 1 (<1%) | 0 | 1 (<1%) |
| Lacrimal Disorders | n (%) | 2 (<1%) | 0 | 2 (<1%) |
| Dry Eye NOS | n (%) | 2 (<1%) | 0 | 2 (<1%) |
| Ocular Nerve and Muscle Disorders | n (%) | 0 | 2 (<1%) | 2 (<1%) |
| Binocular Eye Movement Disorder NOS | n (%) | 0 | 1 (<1%) | 1 (<1%) |
| Strabismus | n (%) | 0 | 1 (<1%) | 1 (<1%) |
| Lid, Lash and Lacrimal Structural Disorders (Excl Congenital) | n (%) | 1 (<1%) | 0 | 1 (<1%) |
| Dacryostenosis Acquired | n (%) | 1 (<1%) | 0 | 1 (<1%) |
| Ocular Disorders NEC | n (%) | 0 | 1 (<1%) | 1 (<1%) |
| Vitreous Disorder NOS | n (%) | 0 | 1 (<1%) | 1 (<1%) |
| Partial Vision Loss | n (%) | 0 | 1 (<1%) | 1 (<1%) |
| Visual Acuity Reduced | n (%) | 0 | 1 (<1%) | 1 (<1%) |
| Refractive and Accommodative Disorders | n (%) | 1 (<1%) | 0 | 1 (<1%) |
| Refractive Errors NOS | n (%) | 1 (<1%) | 0 | 1 (<1%) |
| Retinal Structural Change, Deposit and Degeneration | n (%) | 1 (<1%) | 0 | 1 (<1%) |
| Retinal Detachment | n (%) | 1 (<1%) | 0 | 1 (<1%) |
| Ear and Labyrinth Disorders | n (%) | 6 (2%) | 4 (1%) | 10 (2%) |
| Inner Ear Infections and Inflammations | n (%) | 2 (<1%) | 1 (<1%) | 3 (<1%) |
| Labyrinthitis NOS | n (%) | 2 (<1%) | 1 (<1%) | 3 (<1%) |
| Inner Ear Signs and Symptoms | n (%) | 1 (<1%) | 2 (<1%) | 3 (<1%) |
| Vertigo | n (%) | 1 (<1%) | 1 (<1%) | 2 (<1%) |
| Motion Sickness | n (%) | 0 | 1 (<1%) | 1 (<1%) |
| Tinnitus | n (%) | 1 (<1%) | 0 | 1 (<1%) |
| Ear Disorders NEC | n (%) | 1 (<1%) | 1 (<1%) | 2 (<1%) |
| Ear Pain | n (%) | 1 (<1%) | 1 (<1%) | 2 (<1%) |
| External Ear Disorders NEC | n (%) | 2 (<1%) | 0 | 2 (<1%) |
| Cerumen Impaction | n (%) | 2 (<1%) | 0 | 2 (<1%) |
| Hearing Losses | n (%) | 1 (<1%) | 0 | 1 (<1%) |
| Deafness NOS | n (%) | 1 (<1%) | 0 | 1 (<1%) |
| Inner Ear Disorders NEC | n (%) | 1 (<1%) | 0 | 1 (<1%) |
| Meniere's Disease | n (%) | 1 (<1%) | 0 | 1 (<1%) |
| Middle Ear Disorders NEC | n (%) | 1 (<1%) | 0 | 1 (<1%) |
| Fluid in Middle Ear | n (%) | 1 (<1%) | 0 | 1 (<1%) |
| Tympanic Membrane Disorders (Excl Infections) | n (%) | 0 | 1 (<1%) | 1 (<1%) |
| Tympanic Membrane Disorder NOS | n (%) | 0 | 1 (<1%) | 1 (<1%) |
| Immune System Disorders | n (%) | 5 (2%) | 4 (1%) | 9 (2%) |
| Atopic Disorders | n (%) | 4 (2%) | 1 (<1%) | 5 (<1%) |
| Seasonal Allergy | n (%) | 4 (2%) | 1 (<1%) | 5 (<1%) |
| Allergic Conditions NEC | n (%) | 1 (<1%) | 2 (<1%) | 3 (<1%) |
| Hypersensitivity NOS | n (%) | 1 (<1%) | 1 (<1%) | 2 (<1%) |
| Allergy to Insect Sting | n (%) | 0 | 1 (<1%) | 1 (<1%) |
| Allergies to Foods, Food Additives, Drugs and Other Chemicals | n (%) | 1 (<1%) | 1 (<1%) | 2 (<1%) |
| Drug Hypersensitivity | n (%) | 1 (<1%) | 0 | 1 (<1%) |
| Food Allergy | n (%) | 0 | 1 (<1%) | 1 (<1%) |
| Blood and Lymphatic System Disorders | n (%) | 3 (1%) | 2 (<1%) | 5 (<1%) |
| Leukopenias NEC | n (%) | 2 (<1%) | 0 | 2 (<1%) |
| Leukopenia NOS | n (%) | 2 (<1%) | 0 | 2 (<1%) |
| Lymphangitis and Lymphatic Disorders NEC | n (%) | 1 (<1%) | 1 (<1%) | 2 (<1%) |
| Lymphadenopathy | n (%) | 1 (<1%) | 1 (<1%) | 2 (<1%) |
| Anaemias NEC | n (%) | 0 | 1 (<1%) | 1 (<1%) |
| Anaemia NOS | n (%) | 0 | 1 (<1%) | 1 (<1%) |
| Thrombocytopenias | n (%) | 0 | 1 (<1%) | 1 (<1%) |
| Thrombocytopenia | n (%) | 0 | 1 (<1%) | 1 (<1%) |
| Hepatobiliary Disorders | n (%) | 4 (2%) | 1 (<1%) | 5 (<1%) |
| Cholecystitis and Cholelithiasis | n (%) | 2 (<1%) | 0 | 2 (<1%) |
| Cholelithiasis | n (%) | 2 (<1%) | 0 | 2 (<1%) |
| Hepatobiliary Signs and Symptoms | n (%) | 1 (<1%) | 0 | 1 (<1%) |
| Hepatosplenomegaly NOS | n (%) | 1 (<1%) | 0 | 1 (<1%) |
| Hepatocellular Damage and Hepatitis NEC | n (%) | 1 (<1%) | 0 | 1 (<1%) |
| Hepatitis NOS | n (%) | 1 (<1%) | 0 | 1 (<1%) |
| Structural and Other Bile Duct Disorders | n (%) | 0 | 1 (<1%) | 1 (<1%) |
| Biliary Tract Disorder NOS | n (%) | 0 | 1 (<1%) | 1 (<1%) |
| Endocrine Disorders | n (%) | 2 (<1%) | 2 (<1%) | 4 (<1%) |
| Thyroid Hypofunction Disorders | n (%) | 1 (<1%) | 1 (<1%) | 2 (<1%) |
| Acquired Hypothyroidism | n (%) | 1 (<1%) | 1 (<1%) | 2 (<1%) |
| Thyroid Disorders NEC | n (%) | 1 (<1%) | 0 | 1 (<1%) |
| Goitre | n (%) | 1 (<1%) | 0 | 1 (<1%) |
| Thyroid Neoplasms | n (%) | 0 | 1 (<1%) | 1 (<1%) |
| Thyroid Nodule | n (%) | 0 | 1 (<1%) | 1 (<1%) |
| Surgical and Medical Procedures | n (%) | 1 (<1%) | 1 (<1%) | 2 (<1%) |
| Abdominal Therapeutic Procedures NEC | n (%) | 0 | 1 (<1%) | 1 (<1%) |
| Abdominoplasty | n (%) | 0 | 1 (<1%) | 1 (<1%) |
| Hernia Repairs | n (%) | 1 (<1%) | 0 | 1 (<1%) |
| Hernia Repair NOS | n (%) | 1 (<1%) | 0 | 1 (<1%) |
| Congenital, Familial and Genetic Disorders | n (%) | 0 | 1 (<1%) | 1 (<1%) |
| Inborn Errors of Bilirubin Metabolism | n (%) | 0 | 1 (<1%) | 1 (<1%) |
| Gilbert's Syndrome | n (%) | 0 | 1 (<1%) | 1 (<1%) |
| Pregnancy, Puerperium and Perinatal Conditions | n (%) | 1 (<1%) | 0 | 1 (<1%) |
| Normal Pregnancy, Labour and Delivery* | n (%) | 1 (<1%) | 0 | 1 (<1%) |
| Pregnancy NOS* | n (%) | 1 (<1%) | 0 | 1 (<1%) |
| Social Circumstances | n (%) | 0 | 1 (<1%) | 1 (<1%) |
| Age Related Issues | n (%) | 0 | 1 (<1%) | 1 (<1%) |
| Premenopause* | n (%) | 0 | 1 (<1%) | 1 (<1%) |

Incidence of Treatment-Emergent Adverse Events by Relationship to Study Medication and by Severity

In Study 3007, the overall incidence of TEAEs that were reported as possibly, probably, or unlikely related to study medication was 56%. The most common (≧3% overall) related TEAEs (preferred terms) were constipation (23%), abdominal pain NOS (6%), headache NOS (4%), diarrhea NOS (3%), flatulence (4%), nausea (3%), fecal occult blood positive (3%), and blood CK increased (3%).

Severe TEAEs were reported for 18% of subjects. Most TEAEs were mild or moderate in severity. The only severe TEAEs reported for ≧1% of subjects overall were constipation (17 subjects, 3%), abdominal pain NOS (nine subjects, 2%), and headache NOS (eight subjects, 1%).

Prevalence of Treatment-Emergent Adverse Events by Time Interval

Preferred terms with a prevalence of ≧5% during any time interval in either C/C or P/C subjects (excluding the fifth interval for P/C subjects) are displayed in Table 15.

TABLE 15
Summary of Treatment-Emergent Adverse Events with a Prevalence of ≧5%
During any Three-Month Interval (Safety Population)

| Parameter | Statistic | C/C | P/C | Overall |
|---|---|---|---|---|
| Subjects with any TEAEs | | | | |
| >0-3 months | n of N (%) | 166 of 257 (65) | 164 of 279 (59) | 330 of 536 (62) |
| >3-6 months | n of N (%) | 147 of 255 (58) | 115 of 221 (52) | 262 of 476 (55) |
| >6-9 months | n of N (%) | 118 of 201 (59) | 93 of 187 (50) | 211 of 388 (54) |
| >9-12 months | n of N (%) | 97 of 173 (56) | 81 of 157 (52) | 178 of 330 (54) |
| >12 months | n of N (%) | 91 of 147 (62) | 51 of 55 (93) | 142 of 202 (70) |
| Constipation | | | | |
| >0-3 months | n of N (%) | 41 of 257 (16) | 48 of 279 (17) | 89 of 536 (17) |
| >3-6 months | n of N (%) | 28 of 255 (11) | 16 of 221 (7) | 44 of 476 (9) |
| >6-9 months | n of N (%) | 12 of 201 (6) | 9 of 187 (5) | 21 of 388 (5) |
| >9-12 months | n of N (%) | 3 of 173 (2) | 5 of 157 (3) | 8 of 330 (2) |
| >12 months | n of N (%) | 6 of 147 (4) | 0 of 55 (0) | 6 of 202 (3) |
| Abdominal pain NOS | | | | |
| >0-3 months | n of N (%) | 10 of 257 (4) | 19 of 279 (7) | 29 of 536 (5) |
| >3-6 months | n of N (%) | 7 of 255 (3) | 7 of 221 (3) | 14 of 476 (3) |
| >6-9 months | n of N (%) | 1 of 201 (<1) | 2 of 187 (1) | 3 of 388 (<1) |
| >9-12 months | n of N (%) | 1 of 173 (<1) | 1 of 157 (<1) | 2 of 330 (<1) |
| >12 months | n of N (%) | 2 of 147 (1) | 0 of 55 (0) | 2 of 202 (<1) |
| Fecal occult blood positive | | | | |
| >0-3 months | n of N (%) | 14 of 257 (5) | 5 of 279 (2) | 19 of 536 (4) |
| >3-6 months | n of N (%) | 6 of 255 (2) | 1 of 221 (<1) | 7 of 476 (1) |
| >6-9 months | n of N (%) | 3 of 201 (1) | 3 of 187 (2) | 6 of 388 (2) |
| >9-12 months | n of N (%) | 2 of 173 (1) | 2 of 157 (1) | 4 of 330 (1) |
| >12 months | n of N (%) | 2 of 147 (1) | 0 of 55 (0) | 2 of 202 (<1) |
| Headache NOS | | | | |
| >0-3 months | n of N (%) | 17 of 257 (7) | 9 of 279 (3) | 26 of 536 (5) |
| >3-6 months | n of N (%) | 6 of 255 (2) | 3 of 221 (1) | 9 of 476 (2) |
| >6-9 months | n of N (%) | 0 of 201 (0) | 3 of 187 (2) | 3 of 388 (<1) |
| >9-12 months | n of N (%) | 1 of 173 (<1) | 3 of 157 (2) | 4 of 330 (1) |
| >12 months | n of N (%) | 0 of 147 (0) | 1 of 55 (2) | 1 of 202 (<1) |

C = cilansetron 2 mg TID;
P = placebo
Note:
Percentages are based on the total number of subjects in the Safety Population within each time interval.
Note:
Refer to Section 0 for a definition of TEAEs.
Note:
Only MedDRA preferred terms are presented with a prevalence of ≧5% in any time interval in either C/C or P/C subjects, excluding the fifth interval for P/C subjects due to the limited sample size.

A decrease in the prevalence of TEAEs overall was observed after the first three-months of cilansetron treatment; the overall prevalence then remained stable over the following intervals, except for the last (>12 months) interval (62%, 55%, 54%, 54%, and 70%, respectively). Note that the overall prevalence for the fifth (>12 months) interval is of limited value because of the drop in the number of subjects, which was due to the small number of P/C subjects (55) (C/C subjects: 147) in the last interval. This difference in number of subjects between the two groups limits the value of comparisons of safety data between C/C subjects and P/C subjects in the >12 months interval. The prevalence of constipation overall decreased from 17% during the first three-month interval to 9%, 5%, 2%, and 3% during subsequent intervals, respectively. Decreasing trends were observed for each of the most common individual TEAEs. There was no clinically meaningful increase in the prevalence for any TEAE overall. Of note, 7%, 6%, 2%, and 2% of subjects discontinued during the first, second, third, and fourth three-month interval, respectively, due to AEs (combined categories of “AE and lack of efficacy” and “Baseline complaint/AE”).

Incidence of Special Interest Adverse Events

A total of 371 subjects (69%) experienced an AE of special interest. The most frequently reported (≧5% at the preferred term level) special interest AEs were constipation (24%), abdominal pain NOS (7%), headache (6%), diarrhea NOS (6%), fecal occult blood positive (6%), flatulence (5%), nausea (5%), blood CK positive (5%), upper respiratory tract infection NOS (5%), and urinary tract infection NOS (5%).

Constipation was the most frequently reported TEAE during the study (128 of 536 subjects, 24%). Female subjects had a higher incidence of constipation (28%) compared with male subjects (15%). Of the 128 subjects with constipation, 124 subjects experienced at least one episode reported as related to study medication and 17 subjects (3%) had constipation that was judged as severe. None of the constipation episodes were reported as SAEs. Twenty-eight subjects (5%) discontinued study medication due to constipation and seven subjects experienced constipation that led to dose reduction. Most subjects with a TEAE of constipation reported only one constipation episode (83 subjects, 15%) and were typically mildly (8%) or moderately (7%) bothered by the constipation. The mean longest duration of a constipation episode was 10.4 days, however, the median longest duration was only four days. Thirteen subjects experienced a constipation episode (including the terms constipation and feces hard) that lasted longer than 30 days. The longest duration of a constipation episode was 189 days.

Potential complications of constipation (ischemic colitis, fecal impaction, obstruction, perforation, mega rectum, or colectomy) were identified for two subjects during Studies 3006 and 3007. Eight subjects experienced treatment-emergent rectal hemorrhage.

Incidence of Treatment-Emergent Adverse Events by Gender and by Age

The Safety population included 177 (33%) male and 359 (67%) female subjects. Of the most common TEAEs at the higher level term or preferred term level, higher (≧5% difference) incidence was noted among female subjects compared with male subjects for constipation (28% versus 15%), diarrhea excluding infective (9% versus 3%), nausea and vomiting symptoms (8% versus 3%), sinusitis NOS (9% versus 3%), and urinary tract infections (8% versus 1%). In contrast, blood CK increased was more common in male subjects (12%) compared with female subjects (1%). No other relevant difference in incidences by gender was noted.

The Safety population included 139 subjects (26%) in the 18 to 40 years category, 330 subjects (62%) in the 41 to 64 years category, while only 67 subjects (13%) were 65 years or older. The incidence increased with age for constipation (19%, 23%, and 37% in the three age categories, respectively) and a similar but less apparent trend was seen for abdominal pain NOS (8%, 5%, and 12%) and flatulence, bloating and distension (8%, 5%, and 12%). In contrast, the incidence decreased with age for nausea and vomiting symptoms (9%, 6%, and 1%), sinusitis NOS (9%, 7%, and 3%), and a similar trend was seen for lower respiratory tract and lung infections (4%, 6%, and 1%) and headache NOS (7%, 6%, and 1%). No other relevant difference in incidences by age category was noted.

Other Serious Adverse Events

The overall incidence of treatment-emergent SAEs is presented in Table 16. Twenty-nine subjects (5%) experienced treatment-emergent SAEs. Treatment-emergent SAEs reported for two subjects each included breast cancer NOS, angina pectoris, and chest pain. All other SAEs were experienced by no more than one subject. Eleven of 29 subjects discontinued due to the SAEs. One subject had an SAE reported as probably related to study medication (colitis ischemic) and five subjects had treatment-emergent SAEs reported as unlikely related to study medication (status epilepticus, loss of consciousness, angina pectoris and abdominal pain upper, gastritis NOS, and breast cancer NOS). All other treatment-emergent SAEs were reported as unrelated to study medication. In addition to the 29 subjects who experienced treatment-emergent SAEs, one subject had an SAE that was non-treatment emergent.

In Table 16, gender-specific adverse events are denoted by an “*” in the table, and percentages for gender-specific adverse events are based on the number of subjects in the appropriate gender. Percentages for all other adverse events are based on the total number of subjects in the Safety Population. Subjects are counted at most once within each primary system organ class and preferred term. Adverse events were coded to primary system organ class and preferred term using the MedDRA dictionary, version 5.0. All adverse events with onset date on or after the date of first dose of Cilansetron and up through seven days after the last dose of Cilansetron (if not serious) or up through 30 days after the last dose of Cilansetron (if serious) will be considered as treatment-emergent. Adverse events which are already present during the pre-treatment period but increase in severity during the treatment period will also be considered as treatment-emergent. Primary system organ class and preferred term are sorted by decreasing frequency in the overall column.

TABLE 16
Overall Incidence of Treatment-Emergent Serious Adverse Events (Safety Population)

| | | Treatment Group | | |
|---|---|---|---|---|
| Primary System Organ Class | | Cilansetron 2 mg/ | Placebo/ | |
| Preferred Term | Statistic | Cilansetron 2 mg | Cilansetron 2 mg | Overall |
| Total Number of Subjects in the Safety Population | N | 257 | 279 | 536 |
| Total Number of Male Subjects in the Safety Population | n | 89 (35%) | 88 (32%) | 177 (33%) |
| Total Number of Female Subjects in the Safety Population | n | 168 (65%) | 191 (68%) | 359 (67%) |
| Total Number of Subjects with at Least One Serious Adverse Event | n (%) | 19 (7%) | 10 (4%) | 29 (5%) |
| Neoplasms Benign, Malignant and Unspecified (Incl Cysts and Polyps) | n (%) | 3 (1%) | 3 (1%) | 6 (1%) |
| Breast Cancer NOS* | n (%) | 0 | 2 (1%) | 2 (<1%) |
| Benign Ovarian Tumour* | n (%) | 0 | 1 (<1%) | 1 (<1%) |
| Bone Neoplasm Malignant | n (%) | 1 (<1%) | 0 | 1 (<1%) |
| Fibroma NOS | n (%) | 0 | 1 (<1%) | 1 (<1%) |
| Leiomyoma NOS | n (%) | 1 (<1%) | 0 | 1 (<1%) |
| Ovarian Cancer NOS* | n (%) | 1 (<1%) | 0 | 1 (<1%) |
| Ovarian Cystadenofibroma* | n (%) | 0 | 1 (<1%) | 1 (<1%) |
| Cardiac Disorders | n (%) | 3 (1%) | 1 (<1%) | 4 (<1%) |
| Angina Pectoris | n (%) | 2 (<1%) | 0 | 2 (<1%) |
| Myocardial Infarction | n (%) | 1 (<1%) | 0 | 1 (<1%) |
| Supraventricular Tachycardia | n (%) | 0 | 1 (<1%) | 1 (<1%) |
| Gastrointestinal Disorders | n (%) | 3 (1%) | 1 (<1%) | 4 (<1%) |
| Abdominal Pain Upper | n (%) | 1 (<1%) | 0 | 1 (<1%) |
| Appendicitis | n (%) | 1 (<1%) | 0 | 1 (<1%) |
| Colitis Ischaemic | n (%) | 0 | 1 (<1%) | 1 (<1%) |
| Gastritis NOS | n (%) | 1 (<1%) | 0 | 1 (<1%) |
| Irritable Bowel Syndrome Aggravated | n (%) | 1 (<1%) | 0 | 1 (<1%) |
| Infections and Infestations | n (%) | 1 (<1%) | 2 (<1%) | 3 (<1%) |
| Bronchopneumonia NOS | n (%) | 0 | 1 (<1%) | 1 (<1%) |
| Gastroenteritis NOS | n (%) | 1 (<1%) | 0 | 1 (<1%) |
| Sinusitis Acute NOS | n (%) | 0 | 1 (<1%) | 1 (<1%) |
| Urinary Tract Infection NOS | n (%) | 0 | 1 (<1%) | 1 (<1%) |
| General Disorders and Administration Site Conditions | n (%) | 1 (<1%) | 1 (<1%) | 2 (<1%) |
| Chest Pain | n (%) | 1 (<1%) | 1 (<1%) | 2 (<1%) |
| Hepatobiliary Disorders | n (%) | 2 (<1%) | 0 | 2 (<1%) |
| Cholelithiasis | n (%) | 1 (<1%) | 0 | 1 (<1%) |
| Hepatitis NOS | n (%) | 1 (<1%) | 0 | 1 (<1%) |
| Injury, Poisoning and Procedural Complications | n (%) | 2 (<1%) | 0 | 2 (<1%) |
| Heat Exhaustion | n (%) | 1 (<1%) | 0 | 1 (<1%) |
| Non-Accidental Overdose | n (%) | 1 (<1%) | 0 | 1 (<1%) |
| Nervous System Disorders | n (%) | 2 (<1%) | 0 | 2 (<1%) |
| Loss of Consciousness | n (%) | 1 (<1%) | 0 | 2 (<1%) |
| Status Epilepticus | n (%) | 1 (<1%) | 0 | 1 (<1%) |
| Renal and Urinary Disorders | n (%) | 1 (<1%) | 1 (<1%) | 2 (<1%) |
| Bladder Prolapse | n (%) | 0 | 1 (<1%) | 1 (<1%) |
| Calculus Ureteric | n (%) | 1 (<1%) | 0 | 1 (<1%) |
| Reproductive System and Breast Disorders | n (%) | 2 (<1%) | 0 | 2 (<1%) |
| Adenomyosis* | n (%) | 1 (<1%) | 0 | 1 (<1%) |
| Endometriosis* | n (%) | 1 (<1%) | 0 | 1 (<1%) |
| Uterine Polyp NOS* | n (%) | 1 (<1%) | 0 | 1 (<1%) |
| Vaginal Cyst* | n (%) | 1 (<1%) | 0 | 1 (<1%) |
| Immune System Disorders | n (%) | 0 | 1 (<1%) | 1 (<1%) |
| Food Allergy | n (%) | 0 | 1 (<1%) | 1 (<1%) |
| Pregnancy, Puerperium and Perinatal Conditions | n (%) | 1 (<1%) | 0 | 1 (<1%) |
| Pregnancy NOS* | n (%) | 1 (<1%) | 0 | 1 (<1%) |
| Psychiatric Disorders | n (%) | 0 | 1 (<1%) | 1 (<1%) |
| Depression | n (%) | 0 | 1 (<1%) | 1 (<1%) |
| Hallucination NOS | n (%) | 0 | 1 (<1%) | 1 (<1%) |
| Hallucination, Auditory | n (%) | 0 | 1 (<1%) | 1 (<1%) |
| Psychotic Disorder NOS | n (%) | 0 | 1 (<1%) | 1 (<1%) |
| Suicidal Ideation | n (%) | 0 | 1 (<1%) | 1 (<1%) |
| Respiratory, Thoracic and Mediastinal Disorders | n (%) | 0 | 1 (<1%) | 1 (<1%) |
| Asthma NOS | n (%) | 0 | 1 (<1%) | 1 (<1%) |

Note:
Refer to the Analysis Plan for a list of pre-specified gender-specific adverse events. Gender-specific adverse events are denoted by an “*” in the table.
Note:
Percentages for gender-specific adverse events are based on the number of subjects in the appropriate gender. Percentages for all other adverse events are based on the total number of subjects in the Safety Population.
Note:
Subjects are counted at most once within each primary system organ class and preferred term. Adverse events were coded to primary system organ class and preferred term using the MedDRA dictionary, version 5.0.
Note:
All adverse events with onset date on or after the date of first dose of Cilansetron and up through seven days after the last dose of Cilansetron (if not serious) or up through 30 days after the last dose of Cilansetron (if serious) will be considered as treatment-emergent. Adverse events which are already present during the pre-treatment period but increase in severity during the treatment period will also be considered as treatment-emergent.
Note:
Primary system organ class and preferred term are sorted by decreasing frequency in the overall column.
PROGRAM NAME: S:\SAS\SOLVAY\IBS\CRK00053100\ANALYSIS\TABLELIB\AE_SER.SAS Run 01OCT04 16:13

Other Significant Adverse Events

The overall incidence of TEAEs leading to permanent discontinuation of study medication is presented in Table 17. Overall, 83 subjects (15%) experienced TEAEs that led to permanent discontinuation of study medication with slightly lower incidence in C/C subjects (13%) compared with P/C subjects (18%). A possible explanation for this phenomenon is that subjects who started cilansetron treatment in Study 3006 and discontinued during the first 12 weeks of cilansetron treatment are not accounted for in this analysis. The most frequently reported TEAEs that led to permanent discontinuation of study medication were GI disorders (12%) including constipation (5%), abdominal pain NOS (2%), flatulence (1%), and diarrhea NOS (1%) as the most common events. All other TEAEs that led to discontinuation were experienced by <1% of subjects. Eleven of 83 subjects discontinued due to TEAEs that were reported as serious. In addition to the 83 subjects who discontinued due to TEAEs, two subjects discontinued due to AEs that were non-treatment-emergent: Subject 702020 (constipation) and Subject 703020 (metastases to bone); both events reported with an onset date >30 days after the last dose of cilansetron.

A total of 11 subjects (2%) experienced a TEAEs that led to dose reduction. Of these 11 subjects, seven subjects (1%) had a dose reduction due to constipation.

In Table 17, gender-specific adverse events are denoted by an “*” in the table, and percentages for gender-specific adverse events are based on the number of subjects in the appropriate gender. Percentages for all other adverse events are based on the total number of subjects in the Safety Population. Subjects are counted at most once within each primary system organ class and preferred term. Adverse events were coded to primary system organ class and preferred term using the MedDRA dictionary, version 5.0. All adverse events with onset date on or after the date of first dose of Cilansetron and up through seven days after the last dose of Cilansetron (if not serious) or up through 30 days after the last dose of Cilansetron (if serious) will be considered as treatment-emergent. Adverse events which are already present during the pre-treatment period but increase in severity during the treatment period will also be considered as treatment-emergent. Adverse events leading to permanent discontinuation of Cilansetron were identified from the “Action Study Medication” field of the adverse event case report form. Primary system organ class and preferred term are sorted by decreasing frequency in the overall column.

TABLE 17
Overall Incidence of Treatment-Emergent Adverse Events Leading to Permanent Discontinuation of Study Medication
(Safety Population)

| | | Treatment Group | | |
|---|---|---|---|---|
| Primary System Organ Class | | Cilansetron 2 mg/ | Placebo/ | |
| Preferred Term | Statistic | Cilansetron 2 mg | Cilansetron 2 mg | Overall |
| Total Number of Subjects in the Safety Population | N | 257 | 279 | 536 |
| Total Number of Male Subjects in the Safety Population | n | 89 (35%) | 88 (32%) | 177 (33%) |
| Total Number of Female Subjects in the Safety Population | n | 168 (65%) | 191 (68%) | 359 (67%) |
| Total Number of Subjects with at Least One TEAE Leading to | n (%) | 33 (13%) | 50 (18%) | 83 (15%) |
| Discontinuation of Study Medication | | | | |
| Gastrointestinal Disorders | n (%) | 24 (9%) | 40 (14%) | 64 (12%) |
| Constipation | n (%) | 7 (3%) | 21 (8%) | 28 (5%) |
| Abdominal Pain NOS | n (%) | 5 (2%) | 6 (2%) | 11 (2%) |
| Flatulence | n (%) | 2 (<1%) | 5 (2%) | 7 (1%) |
| Diarrhoea NOS | n (%) | 4 (2%) | 2 (<1%) | 6 (1%) |
| Abdominal Distension | n (%) | 2 (<1%) | 2 (<1%) | 4 (<1%) |
| Abdominal Pain Upper | n (%) | 1 (<1%) | 3 (1%) | 4 (<1%) |
| Rectal Haemorrhage | n (%) | 2 (<1%) | 2 (<1%) | 4 (<1%) |
| Nausea | n (%) | 3 (1%) | 0 | 3 (<1%) |
| Abdominal Pain Lower | n (%) | 0 | 2 (<1%) | 2 (<1%) |
| Diarrhoea Aggravated | n (%) | 1 (<1%) | 1 (<1%) | 2 (<1%) |
| Dyspepsia | n (%) | 1 (<1%) | 1 (<1%) | 2 (<1%) |
| Abdominal Pain Aggravated | n (%) | 1 (<1%) | 0 | 1 (<1%) |
| Appendicitis | n (%) | 1 (<1%) | 0 | 1 (<1%) |
| Colitis Ischaemic | n (%) | 0 | 1 (<1%) | 1 (<1%) |
| Diarrhoea Haemorrhagic | n (%) | 0 | 1 (<1%) | 1 (<1%) |
| Epigastric Discomfort | n (%) | 0 | 1 (<1%) | 1 (<1%) |
| Eructation | n (%) | 1 (<1%) | 0 | 1 (<1%) |
| Faecal Abnormality NOS | n (%) | 1 (<1%) | 0 | 1 (<1%) |
| Faecal Impaction | n (%) | 0 | 1 (<1%) | 1 (<1%) |
| Faecal Incontinence | n (%) | 0 | 1 (<1%) | 1 (<1%) |
| Faeces Hard | n (%) | 1 (<1%) | 0 | 1 (<1%) |
| Gastric Irritation | n (%) | 1 (<1%) | 0 | 1 (<1%) |
| Gastrointestinal Motility Disorder NOS | n (%) | 0 | 1 (<1%) | 1 (<1%) |
| Haemorrhoidal Haemorrhage | n (%) | 0 | 1 (<1%) | 1 (<1%) |
| Hiatus Hernia | n (%) | 1 (<1%) | 0 | 1 (<1%) |
| Irritable Bowel Syndrome Aggravated | n (%) | 1 (<1%) | 0 | 1 (<1%) |
| Oesophageal Reflux Aggravated | n (%) | 1 (<1%) | 0 | 1 (<1%) |
| Rectal Disorder NOS | n (%) | 0 | 1 (<1%) | 1 (<1%) |
| Rectal Tenesmus | n (%) | 0 | 1 (<1%) | 1 (<1%) |
| Stomach Discomfort | n (%) | 0 | 1 (<1%) | 1 (<1%) |
| Vomiting NOS | n (%) | 1 (<1%) | 0 | 1 (<1%) |
| Nervous System Disorders | n (%) | 4 (2%) | 4 (1%) | 8 (1%) |
| Headache NOS | n (%) | 1 (<1%) | 3 (1%) | 4 (<1%) |
| Dizziness | n (%) | 0 | 2 (<1%) | 2 (<1%) |
| Headache NOS Aggravated | n (%) | 1 (<1%) | 0 | 1 (<1%) |
| Migraine NOS | n (%) | 1 (<1%) | 0 | 1 (<1%) |
| Somnolence | n (%) | 1 (<1%) | 0 | 1 (<1%) |
| General Disorders and Administration Site Conditions | n (%) | 3 (1%) | 4 (1%) | 7 (1%) |
| Fatigue | n (%) | 3 (1%) | 1 (<1%) | 4 (<1%) |
| Chest Discomfort | n (%) | 0 | 1 (<1%) | 1 (<1%) |
| Chest Pain | n (%) | 0 | 1 (<1%) | 1 (<1%) |
| Lethargy | n (%) | 0 | 1 (<1%) | 1 (<1%) |
| Musculoskeletal and Connective Tissue Disorders | n (%) | 5 (2%) | 2 (<1%) | 7 (1%) |
| Back Pain | n (%) | 3 (1%) | 1 (<1%) | 4 (<1%) |
| Muscle Cramps | n (%) | 2 (<1%) | 0 | 2 (<1%) |
| Chest Wall Pain | n (%) | 0 | 1 (<1%) | 1 (<1%) |
| Investigations | n (%) | 5 (2%) | 0 | 5 (<1%) |
| Alanine Aminotransferase Increased | n (%) | 1 (<1%) | 0 | 1 (<1%) |
| Blood Bilirubin Increased | n (%) | 1 (<1%) | 0 | 1 (<1%) |
| Blood Pressure Increased | n (%) | 1 (<1%) | 0 | 1 (<1%) |
| Blood Urine Present | n (%) | 1 (<1%) | 0 | 1 (<1%) |
| Heart Rate Increased | n (%) | 1 (<1%) | 0 | 1 (<1%) |
| White Blood Cells Urine Positive | n (%) | 1 (<1%) | 0 | 1 (<1%) |
| Neoplasms Benign, Malignant and Unspecified (Incl Cysts and Polyps) | n (%) | 2 (<1%) | 3 (1%) | 5 (<1%) |
| Breast Cancer NOS* | n (%) | 0 | 2 (1%) | 2 (<1%) |
| Benign Ovarian Tumour* | n (%) | 0 | 1 (<1%) | 1 (<1%) |
| Bone Neoplasm Malignant | n (%) | 1 (<1%) | 0 | 1 (<1%) |
| Fibroma NOS | n (%) | 0 | 1 (<1%) | 1 (<1%) |
| Ovarian Cancer NOS* | n (%) | 1 (<1%) | 0 | 1 (<1%) |
| Ovarian Cystadenofibroma* | n (%) | 0 | 1 (<1%) | 1 (<1%) |
| Skin and Subcutaneous Tissue Disorders | n (%) | 1 (<1%) | 3 (1%) | 4 (<1%) |
| Pruritus NOS | n (%) | 1 (<1%) | 0 | 1 (<1%) |
| Rash Macular | n (%) | 0 | 1 (<1%) | 1 (<1%) |
| Rash NOS | n (%) | 0 | 1 (<1%) | 1 (<1%) |
| Urticaria NOS | n (%) | 0 | 1 (<1%) | 1 (<1%) |
| Respiratory, Thoracic and Mediastinal Disorders | n (%) | 2 (<1%) | 1 (<1%) | 3 (<1%) |
| Asthma Aggravated | n (%) | 1 (<1%) | 0 | 1 (<1%) |
| Asthma NOS | n (%) | 1 (<1%) | 0 | 1 (<1%) |
| Cough | n (%) | 0 | 1 (<1%) | 1 (<1%) |
| Cardiac Disorders | n (%) | 1 (<1%) | 0 | 1 (<1%) |
| Angina Pectoris | n (%) | 1 (<1%) | 0 | 1 (<1%) |
| Ear and Labyrinth Disorders | n (%) | 1 (<1%) | 0 | 1 (<1%) |
| Vertigo | n (%) | 1 (<1%) | 0 | 1 (<1%) |
| Eye Disorders | n (%) | 0 | 1 (<1%) | 1 (<1%) |
| Visual Acuity Reduced | n (%) | 0 | 1 (<1%) | 1 (<1%) |
| Hepatobiliary Disorders | n (%) | 1 (<1%) | 0 | 1 (<1%) |
| Hepatosplenomegaly NOS | n (%) | 1 (<1%) | 0 | 1 (<1%) |
| Metabolism and Nutrition Disorders | n (%) | 1 (<1%) | 0 | 1 (<1%) |
| Dehydration | n (%) | 1 (<1%) | 0 | 1 (<1%) |
| Pregnancy, Puerperium and Perinatal Conditions | n (%) | 1 (<1%) | 0 | 1 (<1%) |
| Pregnancy NOS* | n (%) | 1 (<1%) | 0 | 1 (<1%) |
| Psychiatric Disorders | n (%) | 0 | 1 (<1%) | 1 (<1%) |
| Depression | n (%) | 0 | 1 (<1%) | 1 (<1%) |
| Hallucination NOS | n (%) | 0 | 1 (<1%) | 1 (<1%) |
| Hallucination, Auditory | n (%) | 0 | 1 (<1%) | 1 (<1%) |
| Psychotic Disorder NOS | n (%) | 0 | 1 (<1%) | 1 (<1%) |
| Suicidal Ideation | n (%) | 0 | 1 (<1%) | 1 (<1%) |
| Renal and Urinary Disorders | n (%) | 0 | 1 (<1%) | 1 (<1%) |
| Bladder Prolapse | n (%) | 0 | 1 (<1%) | 1 (<1%) |

Note:
Refer to the Analysis Plan for a list of pre-specified gender-specific adverse events. Gender-specific adverse events are denoted by an “*” in the table.
Note:
Percentages for gender-specific adverse events are based on the number of subjects in the appropriate gender. Percentages for all other adverse events are based on the total number of subjects in the Safety Population.
Note:
Subjects are counted at most once within each primary system organ class and preferred term. Adverse events were coded to primary system organ class and preferred term using the MedDRA dictionary, version 5.0.
Note:
All adverse events with onset date on or after the date of first dose of Cilansetron and up through seven days after the last dose of Cilansetron (if not serious) or up through 30 days after the last dose of Cilansetron (if serious) will be considered as treatment-emergent. Adverse events which are already present during the pre-treatment period but increase in severity during the treatment period will also be considered as treatment-emergent.
Note:
Adverse events leading to permanent discontinuation of Cilansetron were identified from the “Action Study Medication” field of the adverse event case report form.
Note:
Primary system organ class and preferred term are sorted by decreasing frequency in the overall column.
PROGRAM NAME: S:\SAS\SOLVAY\IBS\CRK00053100\ANALYSIS\TABLELIB\AE_DISC.SAS Run 01OCT04 16:13

Treatment-Emergent ECG Abnormalities

A total of 329 subjects (62%) had a treatment-emergent ECG abnormality. Treatment-emergent ECG abnormalities with an incidence ≧5% included sinus arrhythmia (21%), poor R-wave progression (11%), sinus bradycardia (10%), early repolarization-normal variant (8%), non-specific T-wave changes (7%), borderline LA enlargement (6%), and early R-wave transition (6%). Of note, the incidence of prolonged QT interval was 4% and subjects were included in the prolonged QT interval category if they met the following criteria:
Male subjects: QT_{c}(B)>0.450 sec and an increase of 0.06 sec OR QT or QT_{c}(B)>0.500 sec
Female subjects: QT_{c}(B)>0.470 sec and an increase of 0.06 sec OR QT or QT_{c}(B)>0.500 sec

The Safety population included 177 (33%) male and 359 (67%) female subjects. Higher (≧5% difference) incidence was noted among male subjects compared with female subjects for sinus arrhythmia (25% versus 19%) and early repolarization-normal variant (19% versus 2%). In contrast, poor R-wave progression was more common in female subjects (13%) compared with male subjects (6%). No other relevant difference in incidences by gender was noted.

One subject had an ECG abnormality (supraventricular tachycardia) that was considered as a treatment-emergent SAE.

One subject discontinued study medication due to an ECG abnormality (heart rate increased) that was reported as a non-serious TEAE:

Marked Electrocardiogram Abnormalities

The definition of marked abnormalities included PR ≧0.210 sec, QRS ≧0.120 sec, QT_{c} >0.440 sec (regardless of the Baseline value), and heart rate ≧120 bpm or ≦50 bpm or change of ≧15 bpm. A total of 267 subjects (50%) had a post-Baseline marked ECG abnormality. The most frequently reported abnormalities were prolonged QT_{c}(B) (17%), heart rate increase (15%), and heart rate decrease (14%).

The Safety population included 177 (33%) male and 359 (67%) female subjects. Higher (≧5% difference) incidence was noted among female subjects compared with male subjects for prolonged QT_{c}(B) (23% versus 6%) and prolonged QT_{c}(F) (10% versus 3%). In contrast, decreased heart rate was more common in male subjects (13%) compared with female subjects (6%). No other relevant difference in incidences by gender was noted.

3008

Safety Evaluation

The extent of exposure to study medication is presented in Table 18.

TABLE 18
Exposure to Study Medication (Safety Population) - 3008

| | | Cilansetron 2 mg TID |
|---|---|---|
| Parameter | Statistic | (N = 1454) |
| Exposure to study medication (days) | n | 1454 |
| | Mean (SE) | 299.2 (3.4) |
| | Median | 364 |
| | Min-Max | 1-434 |
| Category of exposure | n | 1415 |
| >0-1 month | n (%) | 125 (9) |
| >1-2 months | n (%) | 41 (3) |
| >2-3 months | n (%) | 50 (4) |
| >3-6 months | n (%) | 76 (5) |
| >6-9 months | n (%) | 32 (2) |
| >9 months | n (%) | 1091 (77) |

Note:
Percentages are based on the total number of subjects in the Safety Population with an evaluation of exposure.
Note:
Exposure to study medication is calculated as the date of first dose subtracted from the date of last dose plus 1.
Note:
Exposure to study medication is calculated without accounting for temporary discontinuation of study medication.
Data source: Table 10.1.1.9.2

The mean duration of exposure to study medication was 299.2 days and the median duration was 364 days with 77% of subjects who had more than nine months of exposure to study medication.

Drug Holiday

Eleven percent of subjects had a drug holiday with mean drug holiday duration of 6.19 days. The proportion of subjects who had more than one drug holiday was 1%. The total days on drug holiday accounted for 1.9% of the total duration of exposure for subjects with a drug holiday. The proportion of subjects with a drug holiday increased over the first three one-month intervals (<1%, 2%, and 3%) but decreased for each three-month interval over the course of the study (5%, 4%, 3%, and <1%).

Adverse Events

A summary of TEAEs is presented in Table 19.

| | | Cilansetron |
|---|---|---|
| | Sta- | 2 mg TID |
| Parameter | tistic | (N = 1454) |
| Number of subjects with at least one TEAE | n (%) | 931 (64) |
| Number of subjects with at least one related TEAE | n (%) | 604 (42) |
| Number of subjects with at least one severe TEAE | n (%) | 138 (9) |
| Number of deaths | n (%) | 1 (<1) |
| Number of subjects with at least one SAE | n (%) | 85 (6) |
| Number of subjects who permanently discontinued | n (%) | 192 (13) |
| study medication due to a TEAE | | |
| Number of subjects with at least one TEAE leading | n (%) | 34 (2) |
| to dose reduction | | |
| Number of subjects with at least one AE of | n (%) | 789 (54) |
| special interest | | |

Note:
Percentages are based on the total number of subjects in the Safety Population.
Note:
Refer to Section 5.7.1.10.1 for a definition of TEAEs.
Note:
Related = probably, possibly, or unlikely related; not related = unrelated; unknown = unknown/not applicable.
Note:
Two subjects (115017 and 160003) withdrew from the study due to AEs that were not treatment-emergent; therefore, these subjects and events are not included in the number of subjects who permanently discontinued study medication row.
Data soure: Table 10.1.3.1.1

Sixty-four percent of subjects experienced at least one TEAE during the study. The overall incidence of TEAEs that were reported as possibly, probably, or unlikely related to study medication was 42%. Severe TEAEs were reported for 9%. Six percent of subjects experienced a treatment emergent SAE. Thirteen percent of subjects experienced TEAEs that led to permanent discontinuation of study medication and 2% of subjects experienced TEAEs that led to dose reduction. Special interest TEAEs were reported for 54%.

Analysis of Adverse Events

The overall incidence of TEAEs is presented by primary system organ class, higher level term, and preferred term in Table 20. The incidence of TEAEs occurring in ≧5% of the subjects based on the MedDRA higher level term.

TABLE 20
Overall Incidence of Treatment-Emergent Adverse Events By Primary Organ System,
Higher Level Term, and Preferred Term (Safety Population) - 3008

| Primary System Organ Class/ | | |
|---|---|---|
| Higher Level Term/ | | Treatment Group |
| Preferred Term | Statistic | Cilansetron 2 mg |
| Total Number of Subjects in the Safety Population | N | 1454 |
| Total Number of Male Subjects in the Safety Population | n | 650 |
| Total Number of Female Subjects in the Safety Population | n | 804 |
| Total Number of Subjects with at Least One TEAE | n (%) | 931 (64%) |
| Gastrointestinal Disorders | n (%) | 587 (40%) |
| Gastrointestinal Atonic and Hypomotility Disorders NEC | n (%) | 228 (16%) |
| Constipation | n (%) | 222 (15%) |
| Gastrooesophageal Reflux Disease | n (%) | 7 (<1%) |
| Oesophageal Reflux Aggravated | n (%) | 1 (<1%) |
| Gastrointestinal and Abdominal Pains (Excl Oral and Throat) | n (%) | 223 (15%) |
| Abdominal Pain NOS | n (%) | 156 (11%) |
| Abdominal Pain Upper | n (%) | 50 (3%) |
| Abdominal Pain Lower | n (%) | 21 (1%) |
| Abdominal Pain Aggravated | n (%) | 8 (<1%) |
| Gastrointestinal Pain NOS | n (%) | 2 (<1%) |
| Nausea and Vomiting Symptoms | n (%) | 92 (6%) |
| Nausea | n (%) | 78 (5%) |
| Vomiting NOS | n (%) | 25 (2%) |
| Retching | n (%) | 3 (<1%) |
| Nausea Aggravated | n (%) | 1 (<1%) |
| Regurgitation of Food | n (%) | 1 (<1%) |
| Flatulence, Bloating and Distension | n (%) | 91 (6%) |
| Abdominal Distension | n (%) | 57 (4%) |
| Flatulence | n (%) | 38 (3%) |
| Aerophagia | n (%) | 1 (<1%) |
| Diarrhoea (Excl Infective) | n (%) | 81 (6%) |
| Diarrhoea NOS | n (%) | 73 (5%) |
| Diarrhoea Aggravated | n (%) | 8 (<1%) |
| Diarrhoea Haemorrhagic | n (%) | 1 (<1%) |
| Dyspeptic Signs and Symptoms | n (%) | 59 (4%) |
| Dyspepsia | n (%) | 50 (3%) |
| Eructation | n (%) | 5 (<1%) |
| Dyspepsia Aggravated | n (%) | 4 (<1%) |
| Hyperacidity | n (%) | 4 (<1%) |
| Faeces Abnormal | n (%) | 51 (4%) |
| Faeces Hard | n (%) | 32 (2%) |
| Loose Stools | n (%) | 15 (1%) |
| Faecal Abnormality NOS | n (%) | 3 (<1%) |
| Faeces Discoloured | n (%) | 2 (<1%) |
| Stools Watery | n (%) | 2 (<1%) |
| Haemorrhoids and Gastrointestinal Varices (Excl Oesophageal) | n (%) | 38 (3%) |
| Haemorrhoids | n (%) | 30 (2%) |
| Haemorrhoidal Haemorrhage | n (%) | 9 (<1%) |
| Haemorrhoids Aggravated | n (%) | 1 (<1%) |
| Intestinal Haemorrhages | n (%) | 22 (2%) |
| Rectal Haemorrhage | n (%) | 20 (1%) |
| Anal Haemorrhage | n (%) | 2 (<1%) |
| Gastrointestinal Spastic and Hypermotility Disorders | n (%) | 21 (1%) |
| Irritable Bowel Syndrome Aggravated | n (%) | 13 (<1%) |
| Frequent Bowel Movements | n (%) | 5 (<1%) |
| Defaecation Urgency | n (%) | 1 (<1%) |
| Intestinal Spasm | n (%) | 1 (<1%) |
| Oesophageal Spasm | n (%) | 1 (<1%) |
| Gastritis (Excl Infective) | n (%) | 17 (1%) |
| Gastritis NOS | n (%) | 13 (<1%) |
| Gastritis Aggravated | n (%) | 4 (<1%) |
| Dental Pain and Sensation Disorders | n (%) | 13 (<1%) |
| Toothache | n (%) | 11 (<1%) |
| Dental Discomfort | n (%) | 1 (<1%) |
| Sensitivity of Teeth | n (%) | 1 (<1%) |
| Gastrointestinal Signs and Symptoms NEC | n (%) | 12 (<1%) |
| Abdominal Discomfort | n (%) | 6 (<1%) |
| Epigastric Discomfort | n (%) | 2 (<1%) |
| Halitosis | n (%) | 2 (<1%) |
| Dysphagia | n (%) | 1 (<1%) |
| Hiccups | n (%) | 1 (<1%) |
| Anal and Rectal Disorders NEC | n (%) | 8 (<1%) |
| Anal Fissure | n (%) | 5 (<1%) |
| Anal Discomfort | n (%) | 3 (<1%) |
| Rectal Prolapse | n (%) | 1 (<1%) |
| Anal and Rectal Pains | n (%) | 7 (<1%) |
| Proctalgia | n (%) | 6 (<1%) |
| Painful Defaecation | n (%) | 1 (<1%) |
| Anal and Rectal Signs and Symptoms | n (%) | 7 (<1%) |
| Rectal Tenesmus | n (%) | 5 (<1%) |
| Pruritus Ani | n (%) | 3 (<1%) |
| Oesophagitis (Excl Infective) | n (%) | 6 (<1%) |
| Oesophagitis NOS | n (%) | 4 (<1%) |
| Reflux Oesophagitis | n (%) | 2 (<1%) |
| Gastric Ulcers and Perforation | n (%) | 4 (<1%) |
| Gastric Erosions | n (%) | 2 (<1%) |
| Gastric Ulcer | n (%) | 2 (<1%) |
| Colitis (Excl Infective) | n (%) | 3 (<1%) |
| Colitis Ischaemic | n (%) | 2 (<1%) |
| Colitis NOS | n (%) | 1 (<1%) |
| Gastrointestinal Inflammatory Disorders NEC | n (%) | 3 (<1%) |
| Duodenitis | n (%) | 2 (<1%) |
| Appendicitis | n (%) | 1 (<1%) |
| Oral Dryness and Saliva Altered | n (%) | 3 (<1%) |
| Dry Mouth | n (%) | 3 (<1%) |
| Abdominal Findings Abnormal | n (%) | 2 (<1%) |
| Abdominal Rigidity | n (%) | 1 (<1%) |
| Bowel Sounds Abnormal | n (%) | 1 (<1%) |
| Anal and Rectal Ulcers and Perforation | n (%) | 2 (<1%) |
| Anal Ulcer | n (%) | 1 (<1%) |
| Rectal Ulcer | n (%) | 1 (<1%) |
| Diaphragmatic Hernias | n (%) | 2 (<1%) |
| Hiatus Hernia | n (%) | 2 (<1%) |
| Gastrointestinal Disorders NEC | n (%) | 2 (<1%) |
| Food Poisoning NOS | n (%) | 1 (<1%) |
| Intestinal Mucosal Hypertrophy | n (%) | 1 (<1%) |
| Gastrointestinal Mucosal Dystrophies and Secretion Disorders | n (%) | 2 (<1%) |
| Colonic Polyp | n (%) | 2 (<1%) |
| Gastrointestinal Neoplasms NEC | n (%) | 2 (<1%) |
| Duodenal Polyp | n (%) | 1 (<1%) |
| Gastric Polyps | n (%) | 1 (<1%) |
| Inguinal Hernias | n (%) | 2 (<1%) |
| Inguinal Hernia NOS | n (%) | 2 (<1%) |
| Stomatitis and Ulceration | n (%) | 2 (<1%) |
| Aphthous Stomatitis | n (%) | 1 (<1%) |
| Stomatitis | n (%) | 1 (<1%) |
| Abdominal Hernias, Site Unspecified | n (%) | 1 (<1%) |
| Epigastric Hernia | n (%) | 1 (<1%) |
| Acute and Chronic Pancreatitis | n (%) | 1 (<1%) |
| Pancreatitis Acute | n (%) | 1 (<1%) |
| Anal and Colorectal Neoplasms NEC | n (%) | 1 (<1%) |
| Rectal Polyp | n (%) | 1 (<1%) |
| Benign Oral Cavity Neoplasms | n (%) | 1 (<1%) |
| Mouth Cyst | n (%) | 1 (<1%) |
| Duodenal Ulcers and Perforation | n (%) | 1 (<1%) |
| Duodenal Ulcer | n (%) | 1 (<1%) |
| Gastrointestinal Dyskinetic Disorders | n (%) | 1 (<1%) |
| Gastrointestinal Motility Disorder NOS | n (%) | 1 (<1%) |
| Gingival Disorders NEC | n (%) | 1 (<1%) |
| Gingivitis | n (%) | 1 (<1%) |
| Intestinal Infections | n (%) | 1 (<1%) |
| Antibiotic Associated Colitis | n (%) | 1 (<1%) |
| Non-Mechanical Ileus | n (%) | 1 (<1%) |
| Ileus Paralytic | n (%) | 1 (<1%) |
| Non-Site Specific Gastrointestinal Haemorrhages | n (%) | 1 (<1%) |
| Haematemesis | n (%) | 1 (<1%) |
| Oral Soft Tissue Signs and Symptoms | n (%) | 1 (<1%) |
| Hypoaesthesia Oral | n (%) | 1 (<1%) |
| Tongue Disorders NEC | n (%) | 1 (<1%) |
| Glossitis | n (%) | 1 (<1%) |
| Infections and Infestations | n (%) | 348 (24%) |
| Upper Respiratory Tract Infections - Pathogen Class Unspecified | n (%) | 98 (7%) |
| Upper Respiratory Tract Infection NOS | n (%) | 56 (4%) |
| Sinusitis NOS | n (%) | 25 (2%) |
| Tonsillitis NOS | n (%) | 11 (<1%) |
| Tracheitis NOS | n (%) | 5 (<1%) |
| Sinusitis Acute NOS | n (%) | 2 (<1%) |
| Tonsillitis Acute NOS | n (%) | 2 (<1%) |
| Peritonsillar Abscess | n (%) | 1 (<1%) |
| Pharyngotonsillitis | n (%) | 1 (<1%) |
| Sinusitis Chronic NOS | n (%) | 1 (<1%) |
| Tracheobronchitis | n (%) | 1 (<1%) |
| Lower Respiratory Tract and Lung Infections | n (%) | 66 (5%) |
| Bronchitis NOS | n (%) | 27 (2%) |
| Bronchitis Acute NOS | n (%) | 24 (2%) |
| Lower Respiratory Tract Infection NOS | n (%) | 10 (<1%) |
| Pneumonia NOS | n (%) | 6 (<1%) |
| Bronchitis Chronic NOS | n (%) | 2 (<1%) |
| Bronchopneumonia NOS | n (%) | 1 (<1%) |
| Influenza Viral Infections | n (%) | 51 (4%) |
| Influenza | n (%) | 51 (4%) |
| Rhinoviral Infections | n (%) | 44 (3%) |
| Nasopharyngitis | n (%) | 44 (3%) |
| Urinary Tract Infections | n (%) | 40 (3%) |
| Urinary Tract Infection NOS | n (%) | 27 (2%) |
| Cystitis Acute NOS | n (%) | 5 (<1%) |
| Cystitis NOS | n (%) | 4 (<1%) |
| Pyelonephritis NOS | n (%) | 3 (<1%) |
| Pyelonephritis Acute NOS | n (%) | 1 (<1%) |
| Viral Infections NEC | n (%) | 37 (3%) |
| Viral Infection NOS | n (%) | 26 (2%) |
| Gastroenteritis Viral NOS | n (%) | 5 (<1%) |
| Upper Respiratory Tract Infection Viral NOS | n (%) | 5 (<1%) |
| Pharyngitis Viral NOS | n (%) | 2 (<1%) |
| Viral Diarrhoea | n (%) | 1 (<1%) |
| Gastrointestinal Infections - Pathogen Class Unspecified | n (%) | 30 (2%) |
| Gastroenteritis NOS | n (%) | 24 (2%) |
| Diarrhoea Infectious | n (%) | 4 (<1%) |
| Anal Abscess | n (%) | 1 (<1%) |
| Appendiceal Abscess | n (%) | 1 (<1%) |
| Infections NEC | n (%) | 19 (1%) |
| Respiratory Tract Infection NOS | n (%) | 13 (<1%) |
| Localised Infection | n (%) | 2 (<1%) |
| Abscess Limb | n (%) | 1 (<1%) |
| Abscess NOS | n (%) | 1 (<1%) |
| Infection NOS | n (%) | 1 (<1%) |
| Post Procedural Site Wound Infection | n (%) | 1 (<1%) |
| Ear Infections | n (%) | 13 (<1%) |
| Ear Infection NOS | n (%) | 8 (<1%) |
| Otitis Media NOS | n (%) | 3 (<1%) |
| Otitis Externa NOS | n (%) | 2 (<1%) |
| Ear Lobe Infection NOS | n (%) | 1 (<1%) |
| Candida Infections | n (%) | 10 (<1%) |
| Vaginal Candidiasis* | n (%) | 5 (<1%) |
| Candidal Infection NOS | n (%) | 2 (<1%) |
| Oral Candidiasis | n (%) | 2 (<1%) |
| Intertrigo Candida | n (%) | 1 (<1%) |
| Herpes Viral Infections | n (%) | 9 (<1%) |
| Herpes Zoster | n (%) | 4 (<1%) |
| Herpes Simplex | n (%) | 3 (<1%) |
| Herpes Viral Infection NOS | n (%) | 2 (<1%) |
| Dental Infections - Pathogen Class Unspecified | n (%) | 7 (<1%) |
| Tooth Caries NOS | n (%) | 3 (<1%) |
| Tooth Abscess | n (%) | 2 (<1%) |
| Gingivitis Infection NOS | n (%) | 1 (<1%) |
| Tooth Infection | n (%) | 1 (<1%) |
| Skin Structures and Soft Tissue Infections | n (%) | 7 (<1%) |
| Skin Infection NOS | n (%) | 2 (<1%) |
| Burn Infection | n (%) | 1 (<1%) |
| Cellulitis | n (%) | 1 (<1%) |
| Nail Bed Infection NOS | n (%) | 1 (<1%) |
| Pseudofolliculitis Barbae | n (%) | 1 (<1%) |
| Skin and Subcutaneous Tissue Abscess NOS | n (%) | 1 (<1%) |
| Fungal Infections NEC | n (%) | 5 (<1%) |
| Skin Fungal Infection NOS | n (%) | 4 (<1%) |
| Nail Fungal Infection NOS | n (%) | 1 (<1%) |
| Female Reproductive Tract Infections* | n (%) | 4 (<1%) |
| Vaginitis* | n (%) | 2 (<1%) |
| Oophoritis* | n (%) | 1 (<1%) |
| Pelvic Inflammatory Disease NOS* | n (%) | 1 (<1%) |
| Inflammatory Disorders Following Infection | n (%) | 4 (<1%) |
| Periodontitis | n (%) | 3 (<1%) |
| Post Herpetic Neuralgia | n (%) | 1 (<1%) |
| Sepsis, Bacteraemia and Viraemia | n (%) | 3 (<1%) |
| Viraemia NOS Present | n (%) | 3 (<1%) |
| Chlamydial Infections | n (%) | 2 (<1%) |
| Chlamydial Infection NOS | n (%) | 1 (<1%) |
| Urethritis Non-Specific | n (%) | 1 (<1%) |
| Helminthic Infections NEC | n (%) | 2 (<1%) |
| Helminthic Infection NOS | n (%) | 2 (<1%) |
| Tinea Infections | n (%) | 2 (<1%) |
| Body Tinea | n (%) | 2 (<1%) |
| Tinea Pedis | n (%) | 1 (<1%) |
| Campylobacter Infections | n (%) | 1 (<1%) |
| Campylobacter Gastroenteritis | n (%) | 1 (<1%) |
| Campylobacter Infection | n (%) | 1 (<1%) |
| Cestode Infections | n (%) | 1 (<1%) |
| Taeniasis | n (%) | 1 (<1%) |
| Eye and Eyelid Infections | n (%) | 1 (<1%) |
| Eye Infection NOS | n (%) | 1 (<1%) |
| Helicobacter Infections | n (%) | 1 (<1%) |
| Helicobacter Infection | n (%) | 1 (<1%) |
| Plasmodia Infections | n (%) | 1 (<1%) |
| Malaria NOS | n (%) | 1 (<1%) |
| Streptococcal Infections | n (%) | 1 (<1%) |
| Pharyngitis Streptococcal | n (%) | 1 (<1%) |
| Trichomonas Infections | n (%) | 1 (<1%) |
| Vulvovaginitis Trichomonal* | n (%) | 1 (<1%) |
| Investigations | n (%) | 176 (12%) |
| Liver Function Analyses | n (%) | 36 (2%) |
| Alanine Aminotransferase Increased | n (%) | 19 (1%) |
| Gamma-Glutamyltransferase Increased | n (%) | 19 (1%) |
| Aspartate Aminotransferase Increased | n (%) | 16 (1%) |
| Blood Bilirubin Increased | n (%) | 5 (<1%) |
| Urine Bilirubin Increased | n (%) | 2 (<1%) |
| Bilirubin Urine | n (%) | 1 (<1%) |
| Blood Bilirubin Unconjugated Increased | n (%) | 1 (<1%) |
| Gamma-Glutamyltransferase Decreased | n (%) | 1 (<1%) |
| Liver Function Tests NOS Abnormal | n (%) | 1 (<1%) |
| Skeletal and Cardiac Muscle Analyses | n (%) | 32 (2%) |
| Blood Creatine Phosphokinase Increased | n (%) | 31 (2%) |
| Cardiac Enzymes Increased | n (%) | 1 (<1%) |
| Cholesterol Analyses | n (%) | 25 (2%) |
| Blood Cholesterol Increased | n (%) | 25 (2%) |
| ECG Investigations | n (%) | 25 (2%) |
| Electrocardiogram T Wave Inversion | n (%) | 7 (<1%) |
| Electrocardiogram T Wave Abnormal | n (%) | 4 (<1%) |
| Electrocardiogram St Segment Depression | n (%) | 3 (<1%) |
| Electrocardiogram St Segment Elevation | n (%) | 3 (<1%) |
| Electrocardiogram Abnormal NOS | n (%) | 2 (<1%) |
| Electrocardiogram Change NOS | n (%) | 1 (<1%) |
| Electrocardiogram Pr Interval Prolonged | n (%) | 1 (<1%) |
| Electrocardiogram Pr Shortened | n (%) | 1 (<1%) |
| Electrocardiogram Q Waves Normal | n (%) | 1 (<1%) |
| Electrocardiogram Qt Shortened | n (%) | 1 (<1%) |
| Electrocardiogram T Wave Amplitude Decreased | n (%) | 1 (<1%) |
| Qrs Axis Abnormal | n (%) | 1 (<1%) |
| White Blood Cell Analyses | n (%) | 25 (2%) |
| Eosinophil Count Increased | n (%) | 18 (1%) |
| White Blood Cell Count Decreased | n (%) | 4 (<1%) |
| Eosinophil Percentage Increased | n (%) | 2 (<1%) |
| Neutrophil Count Decreased | n (%) | 2 (<1%) |
| Neutrophil Count Increased | n (%) | 2 (<1%) |
| Neutrophil Percentage Decreased | n (%) | 2 (<1%) |
| Lymphocyte Count Decreased | n (%) | 1 (<1%) |
| Lymphocyte Count Increased | n (%) | 1 (<1%) |
| Lymphocyte Percentage Decreased | n (%) | 1 (<1%) |
| Monocyte Count Increased | n (%) | 1 (<1%) |
| White Blood Cell Count Increased | n (%) | 1 (<1%) |
| Physical Examination Procedures | n (%) | 23 (2%) |
| Weight Increased | n (%) | 13 (<1%) |
| Body Temperature Increased | n (%) | 5 (<1%) |
| Weight Decreased | n (%) | 3 (<1%) |
| Body Temperature Decreased | n (%) | 2 (<1%) |
| Gastrointestinal Histopathology Procedures | n (%) | 22 (2%) |
| Blood in Stool | n (%) | 13 (<1%) |
| Faecal Occult Blood Positive | n (%) | 9 (<1%) |
| Urinalysis NEC | n (%) | 20 (1%) |
| Blood Urine Present | n (%) | 11 (<1%) |
| Protein Urine Present | n (%) | 7 (<1%) |
| Nitrite Urine Present | n (%) | 4 (<1%) |
| White Blood Cells Urine | n (%) | 3 (<1%) |
| Glucose Urine Present | n (%) | 2 (<1%) |
| Red Blood Cells Urine Positive | n (%) | 2 (<1%) |
| White Blood Cells Urine Positive | n (%) | 2 (<1%) |
| Triglyceride Analyses | n (%) | 11 (<1%) |
| Blood Triglycerides Increased | n (%) | 11 (<1%) |
| Metabolism Tests NEC | n (%) | 9 (<1%) |
| Blood Uric Acid Increased | n (%) | 7 (<1%) |
| Urine Ketone Body Present | n (%) | 2 (<1%) |
| Tissue Enzyme Analyses NEC | n (%) | 7 (<1%) |
| Blood Alkaline Phosphatase NOS Increased | n (%) | 7 (<1%) |
| Vascular Tests NEC (Incl Blood Pressure) | n (%) | 7 (<1%) |
| Blood Pressure Increased | n (%) | 5 (<1%) |
| Arterial Pressure NOS Increased | n (%) | 1 (<1%) |
| Blood Pressure | n (%) | 1 (<1%) |
| Mineral and Electrolyte Analyses | n (%) | 4 (<1%) |
| Blood Potassium Increased | n (%) | 3 (<1%) |
| Serum Ferritin Decreased | n (%) | 1 (<1%) |
| Platelet Analyses | n (%) | 3 (<1%) |
| Platelet Count Decreased | n (%) | 2 (<1%) |
| Platelet Count Increased | n (%) | 1 (<1%) |
| Carbohydrate Tolerance Analyses (Incl Diabetes) | n (%) | 2 (<1%) |
| Blood Glucose Increased | n (%) | 2 (<1%) |
| Pituitary Analyses Anterior | n (%) | 2 (<1%) |
| Blood Thyroid Stimulating Hormone Increased | n (%) | 2 (<1%) |
| Red Blood Cell Analyses | n (%) | 2 (<1%) |
| Haemoglobin Decreased | n (%) | 1 (<1%) |
| Red Blood Cell Count Decreased | n (%) | 1 (<1%) |
| Renal Function Analyses | n (%) | 2 (<1%) |
| Blood Urea Increased | n (%) | 2 (<1%) |
| Blood Creatinine Increased | n (%) | 1 (<1%) |
| Haematological Analyses NEC | n (%) | 1 (<1%) |
| Red Blood Cell Sedimentation Rate Increased | n (%) | 1 (<1%) |
| Vitamin Analyses | n (%) | 1 (<1%) |
| Vitamin B12 Decreased | n (%) | 1 (<1%) |
| Nervous System Disorders | n (%) | 155 (11%) |
| Headaches NEC | n (%) | 104 (7%) |
| Headache NOS | n (%) | 100 (7%) |
| Headache NOS Aggravated | n (%) | 2 (<1%) |
| Tension Headaches | n (%) | 2 (<1%) |
| Sinus Headache | n (%) | 1 (<1%) |
| Neurological Signs and Symptoms NEC | n (%) | 30 (2%) |
| Dizziness | n (%) | 28 (2%) |
| Dysgeusia | n (%) | 2 (<1%) |
| Migraine Headaches | n (%) | 7 (<1%) |
| Migraine NOS | n (%) | 6 (<1%) |
| Migraine Aggravated | n (%) | 1 (<1%) |
| Mononeuropathies | n (%) | 6 (<1%) |
| Sciatica | n (%) | 3 (<1%) |
| Carpal Tunnel Syndrome | n (%) | 2 (<1%) |
| Meralgia Paraesthetica | n (%) | 1 (<1%) |
| Sensory Abnormalities NEC | n (%) | 6 (<1%) |
| Neuralgia NOS | n (%) | 4 (<1%) |
| Intercostal Neuralgia | n (%) | 1 (<1%) |
| Restless Leg Syndrome | n (%) | 1 (<1%) |
| Disturbances in Consciousness NEC | n (%) | 5 (<1%) |
| Syncope | n (%) | 4 (<1%) |
| Vasovagal Attack | n (%) | 1 (<1%) |
| Paraesthesias and Dysaesthesias | n (%) | 5 (<1%) |
| Paraesthesia | n (%) | 3 (<1%) |
| Hypoaesthesia | n (%) | 2 (<1%) |
| Burning Sensation NOS | n (%) | 1 (<1%) |
| Central Nervous system Vascular Disorders NEC | n (%) | 2 (<1%) |
| Carotid Artery Atheroma | n (%) | 1 (<1%) |
| Carotid Artery Stenosis | n (%) | 1 (<1%) |
| Lacunar Infarction | n (%) | 1 (<1%) |
| Memory Loss (Excl Dementia) | n (%) | 2 (<1%) |
| Memory Impairment | n (%) | 1 (<1%) |
| Transient Global Amnesia | n (%) | 1 (<1%) |
| Tremor (Excl Congenital) | n (%) | 2 (<1%) |
| Tremor | n (%) | 2 (<1%) |
| Abnormal Sleep-Related Events | n (%) | 1 (<1%) |
| Sleep Talking | n (%) | 1 (<1%) |
| Absence Seizures | n (%) | 1 (<1%) |
| Petit Mal Epilepsy | n (%) | 1 (<1%) |
| Central Nervous System Haemorrhages and Cerebrovascular Accidents | n (%) | 1 (<1%) |
| Cerebral Infarction | n (%) | 1 (<1%) |
| Cervical Spinal Cord and Nerve Root Disorders | n (%) | 1 (<1%) |
| Cervical Root Pain | n (%) | 1 (<1%) |
| Dystonias | n (%) | 1 (<1%) |
| Torticollis | n (%) | 1 (<1%) |
| Facial Cranial Nerve Disorders | n (%) | 1 (<1%) |
| Facial Palsy | n (%) | 1 (<1%) |
| Lumbar Spinal Cord and Nerve Root Disorders | n (%) | 1 (<1%) |
| Radiculitis Lumbosacral | n (%) | 1 (<1%) |
| Narcolepsy and Hypersomnia | n (%) | 1 (<1%) |
| Hypersomnia | n (%) | 1 (<1%) |
| Peripheral Neuropathies NEC | n (%) | 1 (<1%) |
| Peripheral Neuropathy NOS | n (%) | 1 (<1%) |
| Sleep Apnoeas | n (%) | 1 (<1%) |
| Sleep Apnoea Syndrome | n (%) | 1 (<1%) |
| Transient Cerebrovascular Events | n (%) | 1 (<1%) |
| Transient Ischaemic Attack | n (%) | 1 (<1%) |
| Musculoskeletal and Connective Tissue Disorders | n (%) | 129 (9%) |
| Musculoskeletal and Connective Tissue Signs and Symptoms NEC | n (%) | 66 (5%) |
| Back Pain | n (%) | 40 (3%) |
| Pain in Limb | n (%) | 11 (<1%) |
| Neck Pain | n (%) | 8 (<1%) |
| Back Pain Aggravated | n (%) | 2 (<1%) |
| Muscle Spasms | n (%) | 2 (<1%) |
| Musculoskeletal Chest Pain | n (%) | 2 (<1%) |
| Musculoskeletal Discomfort | n (%) | 1 (<1%) |
| Musculoskeletal Pain | n (%) | 1 (<1%) |
| Neck Stiffness | n (%) | 1 (<1%) |
| Joint Related Signs and Symptoms | n (%) | 31 (2%) |
| Arthralgia | n (%) | 29 (2%) |
| Joint Swelling | n (%) | 2 (<1%) |
| Arthralgia Aggravated | n (%) | 1 (<1%) |
| Muscle Pains | n (%) | 12 (<1%) |
| Myalgia | n (%) | 11 (<1%) |
| Polymyalgia | n (%) | 1 (<1%) |
| Muscle Related Signs and Symptoms NEC | n (%) | 8 (<1%) |
| Muscle Cramps | n (%) | 6 (<1%) |
| Muscle Tightness | n (%) | 1 (<1%) |
| Muscle Twitching | n (%) | 1 (<1%) |
| Arthropathies NEC | n (%) | 6 (<1%) |
| Joint Stiffness | n (%) | 2 (<1%) |
| Monoarthritis | n (%) | 2 (<1%) |
| Arthritis NOS | n (%) | 1 (<1%) |
| Polyarthritis | n (%) | 1 (<1%) |
| Soft Tissue Disorders NEC | n (%) | 6 (<1%) |
| Peripheral Swelling | n (%) | 3 (<1%) |
| Groin Pain | n (%) | 2 (<1%) |
| Inguinal Mass | n (%) | 1 (<1%) |
| Metabolic Bone Disorders | n (%) | 4 (<1%) |
| Osteoporosis NOS | n (%) | 4 (<1%) |
| Osteoarthropathies | n (%) | 4 (<1%) |
| Localised Osteoarthritis | n (%) | 2 (<1%) |
| Osteoarthritis Aggravated | n (%) | 1 (<1%) |
| Spinal Osteoarthritis | n (%) | 1 (<1%) |
| Intervertebral Disc Disorders NEC | n (%) | 3 (<1%) |
| Intervertebral Disc Herniation | n (%) | 2 (<1%) |
| Cervical Disc Lesion | n (%) | 1 (<1%) |
| Tendon Disorders | n (%) | 3 (<1%) |
| Tendonitis | n (%) | 3 (<1%) |
| Rheumatoid Arthropathies | n (%) | 2 (<1%) |
| Rheumatoid Arthritis | n (%) | 1 (<1%) |
| Rheumatoid Arthritis Aggravated | n (%) | 1 (<1%) |
| Spondyloarthropathies | n (%) | 2 (<1%) |
| Spondylosis | n (%) | 2 (<1%) |
| Bursal Disorders | n (%) | 1 (<1%) |
| Bursitis | n (%) | 1 (<1%) |
| Cartilage Disorders | n (%) | 1 (<1%) |
| Osteochondrosis | n (%) | 1 (<1%) |
| Connective Tissue Disorders (Excl Le) | n (%) | 1 (<1%) |
| Scleroderma | n (%) | 1 (<1%) |
| Extremity Deformities | n (%) | 1 (<1%) |
| Toe Deformities NOS | n (%) | 1 (<1%) |
| Respiratory, Thoracic and Mediastinal Disorders | n (%) | 91 (6%) |
| Upper Respiratory Tract Signs and Symptoms | n (%) | 36 (2%) |
| Pharyngitis | n (%) | 27 (2%) |
| Catarrh | n (%) | 2 (<1%) |
| Pharyngolaryngeal Pain | n (%) | 2 (<1%) |
| Sneezing | n (%) | 2 (<1%) |
| Hoarseness | n (%) | 1 (<1%) |
| Snoring | n (%) | 1 (<1%) |
| Upper Respiratory Tract Inflammation | n (%) | 1 (<1%) |
| Coughing and Associated Symptoms | n (%) | 28 (2%) |
| Cough | n (%) | 26 (2%) |
| Productive Cough | n (%) | 1 (<1%) |
| Sputum Discoloured | n (%) | 1 (<1%) |
| Bronchospasm and Obstruction | n (%) | 10 (<1%) |
| Asthma NOS | n (%) | 6 (<1%) |
| Asthma Aggravated | n (%) | 2 (<1%) |
| Chronic Obstructive Airways Disease | n (%) | 1 (<1%) |
| Exacerbated | | |
| Wheezing | n (%) | 1 (<1%) |
| Nasal Congestion and Inflammations | n (%) | 10 (<1%) |
| Rhinitis Allergic NOS | n (%) | 4 (<1%) |
| Rhinitis NOS | n (%) | 4 (<1%) |
| Nasal Congestion | n (%) | 2 (<1%) |
| Breathing Abnormalities | n (%) | 7 (<1%) |
| Dyspnoea NOS | n (%) | 5 (<1%) |
| Dyspnoea Exacerbated | n (%) | 2 (<1%) |
| Paranasal Sinus Disorders (Excl Infections and Neoplasms) | n (%) | 6 (<1%) |
| Maxillary Sinusitis | n (%) | 5 (<1%) |
| Sinus Congestion | n (%) | 1 (<1%) |
| Nasal Disorders NEC | n (%) | 5 (<1%) |
| Epistaxis | n (%) | 2 (<1%) |
| Rhinorrhoea | n (%) | 2 (<1%) |
| Nasal Ulcer | n (%) | 1 (<1%) |
| Laryngeal and Adjacent Sites Disorders NEC (Excl Infections and | n (%) | 3 (<1%) |
| Neoplasms) | | |
| Laryngitis NOS | n (%) | 3 (<1%) |
| Pharyngeal Disorders (Excl Infections and Neoplasms) | n (%) | 1 (<1%) |
| Tonsillar Hypertrophy | n (%) | 1 (<1%) |
| Respiratory Tract Disorders NEC | n (%) | 1 (<1%) |
| Respiratory Disorder NOS | n (%) | 1 (<1%) |
| General Disorders and Administration Site Conditions | n (%) | 85 (6%) |
| Asthenic Conditions | n (%) | 38 (3%) |
| Fatigue | n (%) | 20 (1%) |
| Weakness | n (%) | 14 (<1%) |
| Asthenia | n (%) | 2 (<1%) |
| Malaise | n (%) | 2 (<1%) |
| Lethargy | n (%) | 1 (<1%) |
| Febrile Disorders | n (%) | 20 (1%) |
| Pyrexia | n (%) | 20 (1%) |
| Pain and Discomfort NEC | n (%) | 20 (1%) |
| Chest Pain | n (%) | 12 (<1%) |
| Pain NOS | n (%) | 5 (<1%) |
| Distension NOS | n (%) | 3 (<1%) |
| Axillary Pain | n (%) | 1 (<1%) |
| Chest Discomfort | n (%) | 1 (<1%) |
| General Signs and Symptoms NEC | n (%) | 14 (<1%) |
| Influenza Like Illness | n (%) | 8 (<1%) |
| Fall | n (%) | 2 (<1%) |
| Bloody Discharge | n (%) | 1 (<1%) |
| Hangover | n (%) | 1 (<1%) |
| Sensation of Foreign Body NOS | n (%) | 1 (<1%) |
| Thirst | n (%) | 1 (<1%) |
| Oedema NEC | n (%) | 1 (<1%) |
| Oedema Peripheral | n (%) | 1 (<1%) |
| Psychiatric Disorders | n (%) | 74 (5%) |
| Depressive Disorders | n (%) | 28 (2%) |
| Depression | n (%) | 21 (1%) |
| Depression Aggravated | n (%) | 6 (<1%) |
| Major Depressive Disorder NOS | n (%) | 1 (<1%) |
| Anxiety Symptoms | n (%) | 26 (2%) |
| Anxiety | n (%) | 12 (<1%) |
| Stress Symptoms | n (%) | 8 (<1%) |
| Neurosis NOS | n (%) | 5 (<1%) |
| Anxiety Aggravated | n (%) | 1 (<1%) |
| Disturbances in Initiating and Maintaining Sleep | n (%) | 20 (1%) |
| Insomnia | n (%) | 20 (1%) |
| Panic Disorders | n (%) | 4 (<1%) |
| Panic Disorder NOS | n (%) | 4 (<1%) |
| Affect Alterations NEC | n (%) | 2 (<1%) |
| Affect Lability | n (%) | 1 (<1%) |
| Mood Alteration NOS | n (%) | 1 (<1%) |
| Fluctuating Mood Symptoms | n (%) | 2 (<1%) |
| Mood Swings | n (%) | 2 (<1%) |
| Parasomnias | n (%) | 2 (<1%) |
| Nightmare | n (%) | 2 (<1%) |
| Sexual Desire Disorders | n (%) | 2 (<1%) |
| Libido Decreased | n (%) | 1 (<1%) |
| Loss of Libido | n (%) | 1 (<1%) |
| Sleep Disorders NEC | n (%) | 2 (<1%) |
| Sleep Disorder NOS | n (%) | 2 (<1%) |
| Anxiety Disorders NEC (Incl Obsessive Compulsive Disorder) | n (%) | 1 (<1%) |
| Anxiety Disorder | n (%) | 1 (<1%) |
| Dissociative States | n (%) | 1 (<1%) |
| Dissociation | n (%) | 1 (<1%) |
| Fear Symptoms | n (%) | 1 (<1%) |
| Fear of Falling | n (%) | 1 (<1%) |
| Increased Physical Activity Levels | n (%) | 1 (<1%) |
| Restlessness | n (%) | 1 (<1%) |
| Mental Disorders NEC | n (%) | 1 (<1%) |
| Mental Disorder NOS | n (%) | 1 (<1%) |
| Mood Alterations with Depressive Symptoms | n (%) | 1 (<1%) |
| Depressed Mood | n (%) | 1 (<1%) |
| Skin and Subcutaneous Tissue Disorders | n (%) | 70 (5%) |
| Dermatitis and Eczema | n (%) | 16 (1%) |
| Dermatitis Allergic | n (%) | 11 (<1%) |
| Eczema | n (%) | 3 (<1%) |
| Dermatitis NOS | n (%) | 2 (<1%) |
| Dermatitis Contact | n (%) | 1 (<1%) |
| Skin Inflammation NOS | n (%) | 1 (<1%) |
| Pruritus NEC | n (%) | 14 (<1%) |
| Pruritus NOS | n (%) | 11 (<1%) |
| Pruritus Generalised | n (%) | 2 (<1%) |
| Rash Pruritic | n (%) | 1 (<1%) |
| Rashes, Eruptions and Exanthems NEC | n (%) | 13 (<1%) |
| Rash NOS | n (%) | 10 (<1%) |
| Rash Generalised | n (%) | 3 (<1%) |
| Skin Eruption | n (%) | 1 (<1%) |
| Apocrine and Eccrine Gland Disorders | n (%) | 8 (<1%) |
| Sweating Increased | n (%) | 7 (<1%) |
| Heat Rash | n (%) | 1 (<1%) |
| Alopecias | n (%) | 6 (<1%) |
| Hypotrichosis | n (%) | 4 (<1%) |
| Alopecia | n (%) | 1 (<1%) |
| Alopecia Areata | n (%) | 1 (<1%) |
| Papulosquamous Conditions | n (%) | 6 (<1%) |
| Psoriasis | n (%) | 5 (<1%) |
| Rash Papular | n (%) | 1 (<1%) |
| Dermal and Epidermal Conditions NEC | n (%) | 4 (<1%) |
| Dry Skin | n (%) | 1 (<1%) |
| Skin Disorder NOS | n (%) | 1 (<1%) |
| Skin Necrosis | n (%) | 1 (<1%) |
| Skin Odour Abnormal | n (%) | 1 (<1%) |
| Urticarias | n (%) | 4 (<1%) |
| Urticaria NOS | n (%) | 4 (<1%) |
| Skin Injuries and Mechanical Dermatoses | n (%) | 3 (<1%) |
| Contusion | n (%) | 2 (<1%) |
| Scar | n (%) | 1 (<1%) |
| Acnes | n (%) | 2 (<1%) |
| Acne Aggravated | n (%) | 1 (<1%) |
| Acne NOS | n (%) | 1 (<1%) |
| Nail and Nail Bed Conditions (Excl Infections and Infestations) | n (%) | 2 (<1%) |
| Nail Discolouration | n (%) | 1 (<1%) |
| Nail Disorder NOS | n (%) | 1 (<1%) |
| Onychoschizia | n (%) | 1 (<1%) |
| Connective Tissue Disorders | n (%) | 1 (<1%) |
| Discoid Lupus Erythematosus | n (%) | 1 (<1%) |
| Erythemas | n (%) | 1 (<1%) |
| Erythema | n (%) | 1 (<1%) |
| Photosensitivity Conditions | n (%) | 1 (<1%) |
| Photodermatosis | n (%) | 1 (<1%) |
| Rosaceas | n (%) | 1 (<1%) |
| Rosacea | n (%) | 1 (<1%) |
| Scalar Conditions | n (%) | 1 (<1%) |
| Dandruff | n (%) | 1 (<1%) |
| Sebaceous Gland Disorders | n (%) | 1 (<1%) |
| Seborrhoea | n (%) | 1 (<1%) |
| Skin Neoplasms Benign | n (%) | 1 (<1%) |
| Epidermal Cyst | n (%) | 1 (<1%) |
| Skin Preneoplastic Conditions NEC | n (%) | 1 (<1%) |
| Solar Keratosis | n (%) | 1 (<1%) |
| Skin and Subcutaneous Tissue Bacterial Infections | n (%) | 1 (<1%) |
| Pyoderma | n (%) | 1 (<1%) |
| Skin and Subcutaneous Tissue Ulcerations | n (%) | 1 (<1%) |
| Skin Ulcer | n (%) | 1 (<1%) |
| Injury, Poisoning and Procedural Complications | n (%) | 59 (4%) |
| Site Specific Injuries NEC | n (%) | 17 (1%) |
| Limb Injury NOS | n (%) | 8 (<1%) |
| Back Injury NOS | n (%) | 3 (<1%) |
| Head Injury | n (%) | 3 (<1%) |
| Face Injury | n (%) | 2 (<1%) |
| Anal Injury | n (%) | 1 (<1%) |
| Neck Injury NOS | n (%) | 1 (<1%) |
| Muscle, Tendon and Ligament Injuries | n (%) | 14 (<1%) |
| Muscle Strain | n (%) | 7 (<1%) |
| Ligament Injury NOS | n (%) | 4 (<1%) |
| Muscle Injury NOS | n (%) | 2 (<1%) |
| Tendon Injury | n (%) | 1 (<1%) |
| Non-Site Specific Injuries NEC | n (%) | 10 (<1%) |
| Animal Bite | n (%) | 2 (<1%) |
| Arthropod Sting | n (%) | 2 (<1%) |
| Laceration | n (%) | 2 (<1%) |
| Abrasion NOS | n (%) | 1 (<1%) |
| Injury NOS | n (%) | 1 (<1%) |
| Post-Traumatic Wound Infection | n (%) | 1 (<1%) |
| Road Traffic Accident | n (%) | 1 (<1%) |
| Soft Tissue Injury NOS | n (%) | 1 (<1%) |
| Upper Limb Fractures and Dislocations | n (%) | 7 (<1%) |
| Radius Fracture | n (%) | 2 (<1%) |
| Wrist Fracture | n (%) | 2 (<1%) |
| Clavicle Fracture | n (%) | 1 (<1%) |
| Forearm Fracture | n (%) | 1 (<1%) |
| Hand Fracture | n (%) | 1 (<1%) |
| Non-Site Specific Procedural Complications | n (%) | 5 (<1%) |
| Post Procedural Pain | n (%) | 4 (<1%) |
| Post Procedural Haemorrhage | n (%) | 1 (<1%) |
| Limb Injuries NEC (Incl Traumatic Amputation) | n (%) | 4 (<1%) |
| Joint Sprain | n (%) | 3 (<1%) |
| Joint Dislocation | n (%) | 1 (<1%) |
| Lower Limb Fractures and Dislocations | n (%) | 4 (<1%) |
| Foot Fracture | n (%) | 2 (<1%) |
| Ankle Fracture | n (%) | 1 (<1%) |
| Fibula Fracture | n (%) | 1 (<1%) |
| Thoracic Cage Fractures and Dislocations | n (%) | 2 (<1%) |
| Rib Fracture | n (%) | 2 (<1%) |
| Chest and Lung Injuries NEC | n (%) | 1 (<1%) |
| Traumatic Chest Injury NOS | n (%) | 1 (<1%) |
| Eye Injuries NEC | n (%) | 1 (<1%) |
| Foreign Body in Eye | n (%) | 1 (<1%) |
| Thermal Burns | n (%) | 1 (<1%) |
| Thermal Burn | n (%) | 1 (<1%) |
| Metabolism and Nutrition Disorders | n (%) | 54 (4%) |
| Appetite Disorders | n (%) | 15 (1%) |
| Anorexia | n (%) | 11 (<1%) |
| Appetite Decreased NOS | n (%) | 2 (<1%) |
| Appetite Increased NOS | n (%) | 2 (<1%) |
| Elevated Cholesterol | n (%) | 9 (<1%) |
| Hypercholesterolaemia | n (%) | 8 (<1%) |
| Hypercholesterolaemia Aggravated | n (%) | 1 (<1%) |
| Elevated Triglycerides | n (%) | 8 (<1%) |
| Hypertriglyceridaemia | n (%) | 8 (<1%) |
| Diabetes Mellitus (Incl Subtypes) | n (%) | 4 (<1%) |
| Diabetes Mellitus NOS | n (%) | 3 (<1%) |
| Diabetes Mellitus Non-Insulin-Dependent | n (%) | 1 (<1%) |
| Hyperglycaemic Conditions NEC | n (%) | 4 (<1%) |
| Hyperglycaemia NOS | n (%) | 4 (<1%) |
| Potassium Imbalance | n (%) | 4 (<1%) |
| Hypokalaemia | n (%) | 3 (<1%) |
| Hyperkalaemia | n (%) | 1 (<1%) |
| General Nutritional Disorders NEC | n (%) | 3 (<1%) |
| Hunger | n (%) | 1 (<1%) |
| Overweight | n (%) | 1 (<1%) |
| Weight Fluctuation | n (%) | 1 (<1%) |
| Hyperlipidaemias NEC | n (%) | 3 (<1%) |
| Hyperlipidaemia NOS | n (%) | 3 (<1%) |
| Water Soluble Vitamin Deficiencies | n (%) | 2 (<1%) |
| Vitamin B12 Deficiency | n (%) | 2 (<1%) |
| Food Malabsorption and Intolerance Syndromes (Excl Sugar | n (%) | 1 (<1%) |
| Intolerance) | | |
| Food Intolerance NOS | n (%) | 1 (<1%) |
| Purine Metabolism Disorders NEC | n (%) | 1 (<1%) |
| Gout | n (%) | 1 (<1%) |
| Sodium Imbalance | n (%) | 1 (<1%) |
| Hyponatraemia Aggravated | n (%) | 1 (<1%) |
| Total Fluid Volume Increased | n (%) | 1 (<1%) |
| Fluid Retention | n (%) | 1 (<1%) |
| Cardiac Disorders | n (%) | 46 (3%) |
| Supraventricular Arrhythmias | n (%) | 18 (1%) |
| Sinus Tachycardia | n (%) | 9 (<1%) |
| Sinus Bradycardia | n (%) | 3 (<1%) |
| Atrial Fibrillation | n (%) | 2 (<1%) |
| Sinus Arrhythmia | n (%) | 2 (<1%) |
| Atrial Fibrillation Aggravated | n (%) | 1 (<1%) |
| Atrial Flutter | n (%) | 1 (<1%) |
| Supraventricular Extrasystoles | n (%) | 1 (<1%) |
| Rate and Rhythm Disorders NEC | n (%) | 8 (<1%) |
| Tachycardia NOS | n (%) | 3 (<1%) |
| Bradycardia NOS | n (%) | 2 (<1%) |
| Extrasystoles NOS | n (%) | 2 (<1%) |
| Tachycardia Paroxysmal NOS | n (%) | 1 (<1%) |
| Ischaemic Coronary Artery Disorders | n (%) | 7 (<1%) |
| Myocardial Ischaemia | n (%) | 4 (<1%) |
| Acute Myocardial Infarction | n (%) | 2 (<1%) |
| Angina Pectoris | n (%) | 2 (<1%) |
| Cardiac Signs and Symptoms NEC | n (%) | 5 (<1%) |
| Palpitations | n (%) | 5 (<1%) |
| Cardiac Conduction Disorders | n (%) | 4 (<1%) |
| Bundle Branch Block Right | n (%) | 3 (<1%) |
| Atrioventricular Block NOS | n (%) | 1 (<1%) |
| Ventricular Arrhythmias and Cardiac Arrest | n (%) | 3 (<1%) |
| Ventricular Extrasystoles | n (%) | 3 (<1%) |
| Coronary Artery Disorders NEC | n (%) | 2 (<1%) |
| Coronary Artery Disease NOS | n (%) | 2 (<1%) |
| Myocardial Disorders NEC | n (%) | 2 (<1%) |
| Atrial Hypertrophy | n (%) | 1 (<1%) |
| Ventricular Hypertrophy | n (%) | 1 (<1%) |
| Heart Failure Signs and Symptoms | n (%) | 1 (<1%) |
| Hepatojugular Reflux | n (%) | 1 (<1%) |
| Renal and Urinary Disorders | n (%) | 40 (3%) |
| Bladder and Urethral Symptoms | n (%) | 18 (1%) |
| Dysuria | n (%) | 5 (<1%) |
| Urinary Frequency | n (%) | 5 (<1%) |
| Urinary Incontinence | n (%) | 4 (<1%) |
| Difficulty in Micturition | n (%) | 2 (<1%) |
| Stress Incontinence | n (%) | 2 (<1%) |
| Urinary Incontinence Aggravated | n (%) | 1 (<1%) |
| Urinary Abnormalities | n (%) | 8 (<1%) |
| Haematuria | n (%) | 2 (<1%) |
| Leukocyturia | n (%) | 2 (<1%) |
| Proteinuria | n (%) | 2 (<1%) |
| Chromaturia | n (%) | 1 (<1%) |
| Glycosuria | n (%) | 1 (<1%) |
| Urine Odour Abnormal | n (%) | 1 (<1%) |
| Urinary Tract Signs and Symptoms NEC | n (%) | 8 (<1%) |
| Renal Colic | n (%) | 4 (<1%) |
| Polyuria | n (%) | 2 (<1%) |
| Loin Pain | n (%) | 1 (<1%) |
| Nocturia | n (%) | 1 (<1%) |
| Renal Lithiasis | n (%) | 2 (<1%) |
| Calculus Renal NOS | n (%) | 2 (<1%) |
| Urinary Tract Lithiasis (Excl Renal) | n (%) | 2 (<1%) |
| Calculus Ureteric | n (%) | 2 (<1%) |
| Bladder Disorders NEC | n (%) | 1 (<1%) |
| Cystocele* | n (%) | 1 (<1%) |
| Renal Neoplasms | n (%) | 1 (<1%) |
| Renal Cyst NOS | n (%) | 1 (<1%) |
| Structural and Obstructive Urethral Disorders (Excl Congenital) | n (%) | 1 (<1%) |
| Urethral Stricture | n (%) | 1 (<1%) |
| Reproductive System and Breast Disorders | n (%) | 38 (3%) |
| Breast Signs and Symptoms* | n (%) | 4 (<1%) |
| Breast Pain* | n (%) | 2 (<1%) |
| Breast Engorgement* | n (%) | 1 (<1%) |
| Breast Tenderness* | n (%) | 1 (<1%) |
| Menstruation with Decreased Bleeding* | n (%) | 4 (<1%) |
| Amenorrhoea NOS* | n (%) | 2 (<1%) |
| Oligomenorrhoea NOS* | n (%) | 2 (<1%) |
| Prostatic signs, Symptoms and Disorders NEC* | n (%) | 4 (<1%) |
| Benign Prostatic Hyperplasia* | n (%) | 3 (<1%) |
| Prostatic Pain* | n (%) | 1 (<1%) |
| Menstruation and Uterine Bleeding NEC* | n (%) | 3 (<1%) |
| Dysmenorrhoea* | n (%) | 2 (<1%) |
| Menses Delayed* | n (%) | 1 (<1%) |
| Prostate and Seminal Vesicles Infections and Inflammations* | n (%) | 3 (<1%) |
| Prostatitis* | n (%) | 3 (<1%) |
| Benign and Malignant Breast Neoplasms* | n (%) | 2 (<1%) |
| Breast Cyst* | n (%) | 2 (<1%) |
| Erection and Ejaculation Conditions and Disorders* | n (%) | 2 (<1%) |
| Erectile Dysfunction NOS* | n (%) | 2 (<1%) |
| Menopausal Effects NEC* | n (%) | 2 (<1%) |
| Menopausal Symptoms* | n (%) | 1 (<1%) |
| Postmenopausal Symptoms* | n (%) | 1 (<1%) |
| Menstruation with Increased Bleeding* | n (%) | 2 (<1%) |
| Menorrhagia* | n (%) | 1 (<1%) |
| Polymenorrhoea* | n (%) | 1 (<1%) |
| Ovarian and Fallopian Tube Cysts and Neoplasms* | n (%) | 2 (<1%) |
| Ovarian Cyst* | n (%) | 2 (<1%) |
| Pelvic Prolapse* | n (%) | 2 (<1%) |
| Uterine Prolapse* | n (%) | 1 (<1%) |
| Vaginal Prolapse* | n (%) | 1 (<1%) |
| Reproductive Tract Signs and Symptoms NEC | n (%) | 2 (<1%) |
| Genital Discharge | n (%) | 1 (<1%) |
| Genital Pain NOS | n (%) | 1 (<1%) |
| Breast Disorders NEC* | n (%) | 1 (<1%) |
| Gynaecomastia* | n (%) | 1 (<1%) |
| Cervix Disorders NEC* | n (%) | 1 (<1%) |
| Uterine Cervical Erosion* | n (%) | 1 (<1%) |
| Menopausal Effects on the Genitourinary Tract* | n (%) | 1 (<1%) |
| Postmenopausal Haemorrhage* | n (%) | 1 (<1%) |
| Reproductive Tract Disorders NEC (Excl Neoplasms) | n (%) | 1 (<1%) |
| Genital Haemorrhage NOS | n (%) | 1 (<1%) |
| Scrotal Disorders NEC* | n (%) | 1 (<1%) |
| Scrotal Swelling* | n (%) | 1 (<1%) |
| Vulvovaginal Disorders NEC* | n (%) | 1 (<1%) |
| Vaginal Haemorrhage* | n (%) | 1 (<1%) |
| Vascular Disorders | n (%) | 32 (2%) |
| Vascular Hypertensive Disorders NEC | n (%) | 20 (1%) |
| Hypertension NOS | n (%) | 15 (1%) |
| Hypertension Aggravated | n (%) | 4 (<1%) |
| Essential Hypertension | n (%) | 1 (<1%) |
| Phlebitis NEC | n (%) | 4 (<1%) |
| Phlebitis NOS | n (%) | 2 (<1%) |
| Periphlebitis | n (%) | 1 (<1%) |
| Phlebitis Superficial | n (%) | 1 (<1%) |
| Vascular Hypotensive Disorders | n (%) | 3 (<1%) |
| Orthostatic Hypotension | n (%) | 3 (<1%) |
| Bruising, Ecchymosis and Purpura | n (%) | 1 (<1%) |
| Petechiae | n (%) | 1 (<1%) |
| Circulatory Collapse and Shock | n (%) | 1 (<1%) |
| Shock | n (%) | 1 (<1%) |
| Non-Site Specific Vascular Disorders NEC | n (%) | 1 (<1%) |
| Vascular Disorder NOS | n (%) | 1 (<1%) |
| Peripheral Embolism and Thrombosis | n (%) | 1 (<1%) |
| Venous Thrombosis Deep Limb | n (%) | 1 (<1%) |
| Varicose Veins Non-Site Specific | n (%) | 1 (<1%) |
| Varicose Veins NOS | n (%) | 1 (<1%) |
| Immune System Disorders | n (%) | 23 (2%) |
| Atopic Disorders | n (%) | 12 (<1%) |
| Seasonal Allergy | n (%) | 12 (<1%) |
| Allergic Conditions NEC | n (%) | 8 (<1%) |
| Hypersensitivity NOS | n (%) | 7 (<1%) |
| Allergy to Insect Sting | n (%) | 1 (<1%) |
| Allergies to Foods, Food Additives, Drugs and Other Chemicals | n (%) | 3 (<1%) |
| Drug Hypersensitivity | n (%) | 3 (<1%) |
| Ear and Labyrinth Disorders | n (%) | 22 (2%) |
| Inner Ear Signs and Symptoms | n (%) | 18 (1%) |
| Vertigo | n (%) | 10 (<1%) |
| Tinnitus | n (%) | 8 (<1%) |
| Vertigo Positional | n (%) | 1 (<1%) |
| Ear Disorders NEC | n (%) | 3 (<1%) |
| Ear Pain | n (%) | 3 (<1%) |
| Hyperacusia | n (%) | 1 (<1%) |
| Hyperacusis | n (%) | 1 (<1%) |
| Inner Ear Disorders NEC | n (%) | 1 (<1%) |
| Inner Ear Disorder NOS | n (%) | 1 (<1%) |
| Eye Disorders | n (%) | 18 (1%) |
| Visual Disorders NEC | n (%) | 3 (<1%) |
| Visual Disturbance NOS | n (%) | 3 (<1%) |
| Lid, Lash and Lacrimal Infections, Irritations and Inflammations | n (%) | 2 (<1%) |
| Eyelid Oedema | n (%) | 2 (<1%) |
| Ocular Disorders NEC | n (%) | 2 (<1%) |
| Eye Pain | n (%) | 2 (<1%) |
| Partial Vision Loss | n (%) | 2 (<1%) |
| Vision Abnormal NOS Exacerbated | n (%) | 1 (<1%) |
| Vision Blurred | n (%) | 1 (<1%) |
| Blindness (Excl Colour Blindness) | n (%) | 1 (<1%) |
| Amaurosis Fugax | n (%) | 1 (<1%) |
| Cataracts (Excl Congenital) | n (%) | 1 (<1%) |
| Cataract Unilateral | n (%) | 1 (<1%) |
| Conjunctival Infections, Irritations and Inflammations | n (%) | 1 (<1%) |
| Conjunctivitis | n (%) | 1 (<1%) |
| Conjunctival and Corneal Bleeding and Vascular Disorders | n (%) | 1 (<1%) |
| Conjunctival Haemorrhage | n (%) | 1 (<1%) |
| Glaucomas (Excl Congenital) | n (%) | 1 (<1%) |
| Glaucoma NOS | n (%) | 1 (<1%) |
| Lacrimal Disorders | n (%) | 1 (<1%) |
| Dry Eye NOS | n (%) | 1 (<1%) |
| Ocular Infections, Inflammations and Associated Manifestations | n (%) | 1 (<1%) |
| Eye Irritation | n (%) | 1 (<1%) |
| Ocular Sensation Disorders | n (%) | 1 (<1%) |
| Photophobia | n (%) | 1 (<1%) |
| Retinal Bleeding and Vascular Disorders (Excl Retinopathy) | n (%) | 1 (<1%) |
| Retinal Vascular Disorder NOS | n (%) | 1 (<1%) |
| Retinal Structural Change, Deposit and Degeneration | n (%) | 1 (<1%) |
| Retinal Detachment | n (%) | 1 (<1%) |
| Retinal, Choroid and Vitreous Infections and Inflammations | n (%) | 1 (<1%) |
| Retinitis NOS | n (%) | 1 (<1%) |
| Neoplasms Benign, Malignant and Unspecified (Incl Cysts and Polyps) | n (%) | 17 (1%) |
| Uterine Neoplasms Benign* | n (%) | 3 (<1%) |
| Uterine Fibroids* | n (%) | 3 (<1%) |
| Hepatobiliary Neoplasms Benign | n (%) | 2 (<1%) |
| Haemangioma of Liver | n (%) | 2 (<1%) |
| Non-Small Cell Neoplasms Malignant of the Respiratory Tract Cell | n (%) | 2 (<1%) |
| Type Specified | | |
| Large Cell Carcinoma of the Respiratory | n (%) | 1 (<1%) |
| Tract Stage Unspecified | | |
| Lung Adenocarcinoma NOS | n (%) | 1 (<1%) |
| Soft Tissue Neoplasms Benign NEC | n (%) | 2 (<1%) |
| Lipoma NOS | n (%) | 2 (<1%) |
| Breast and Nipple Neoplasms Malignant* | n (%) | 1 (<1%) |
| Breast Cancer NOS* | n (%) | 1 (<1%) |
| Cartilage Neoplasms Benign | n (%) | 1 (<1%) |
| Enchondromatosis | n (%) | 1 (<1%) |
| Lip and Oral Cavity Neoplasms Benign | n (%) | 1 (<1%) |
| Salivary Gland Adenoma | n (%) | 1 (<1%) |
| Non-Hodgkin's Lymphomas NEC | n (%) | 1 (<1%) |
| Non-Hodgkin's Lymphoma NOS | n (%) | 1 (<1%) |
| Pancreatic Neoplasms Malignant (Excl Islet Cell and Carcinoid) | n (%) | 1 (<1%) |
| Pancreatic Carcinoma NOS | n (%) | 1 (<1%) |
| Rectal Neoplasms Malignant | n (%) | 1 (<1%) |
| Rectal Cancer NOS | n (%) | 1 (<1%) |
| Urinary Tract Neoplasms Benign | n (%) | 1 (<1%) |
| Benign Ureteric Neoplasm NOS | n (%) | 1 (<1%) |
| Urinary Tract Neoplasms Unspecified Malignancy NEC | n (%) | 1 (<1%) |
| Bladder Neoplasm NOS | n (%) | 1 (<1%) |
| Blood and Lymphatic System Disorders | n (%) | 13 (<1%) |
| Anaemias NEC | n (%) | 7 (<1%) |
| Anaemia NOS | n (%) | 5 (<1%) |
| Hypochromic Anaemia | n (%) | 1 (<1%) |
| Normochromic Normocytic Anaemia | n (%) | 1 (<1%) |
| Leukocytoses NEC | n (%) | 4 (<1%) |
| Eosinophilia | n (%) | 2 (<1%) |
| Leukocytosis | n (%) | 2 (<1%) |
| Neutrophilia | n (%) | 2 (<1%) |
| Leukopenias NEC | n (%) | 2 (<1%) |
| Leukopenia NOS | n (%) | 2 (<1%) |
| Anaemia Deficiencies | n (%) | 1 (<1%) |
| Anaemia Vitamin B12 Deficiency | n (%) | 1 (<1%) |
| Neutropenias | n (%) | 1 (<1%) |
| Neutropenia | n (%) | 1 (<1%) |
| Red Blood Cell Abnormal Findings NEC | n (%) | 1 (<1%) |
| Anisocytosis | n (%) | 1 (<1%) |
| Poikilocytosis | n (%) | 1 (<1%) |
| Secondary Thrombocythaemias | n (%) | 1 (<1%) |
| Thrombocythaemia | n (%) | 1 (<1%) |
| Hepatobiliary Disorders | n (%) | 13 (<1%) |
| Hepatocellular Damage and Hepatitis NEC | n (%) | 5 (<1%) |
| Hepatitis NOS | n (%) | 3 (<1%) |
| Liver Fatty | n (%) | 2 (<1%) |
| Cholecystitis and Cholelithiasis | n (%) | 3 (<1%) |
| Cholecystitis NOS | n (%) | 2 (<1%) |
| Cholelithiasis | n (%) | 1 (<1%) |
| Hepatic and Hepatobiliary Disorders NEC | n (%) | 2 (<1%) |
| Hepatic Disorder NOS | n (%) | 2 (<1%) |
| Cholestasis and Jaundice | n (%) | 1 (<1%) |
| Jaundice Cholestatic | n (%) | 1 (<1%) |
| Gallbladder Disorders NEC | n (%) | 1 (<1%) |
| Biliary Dyskinesia | n (%) | 1 (<1%) |
| Hepatobiliary Neoplasms Benign | n (%) | 1 (<1%) |
| Hepatic Cyst NOS | n (%) | 1 (<1%) |
| Endocrine Disorders | n (%) | 7 (<1%) |
| Thyroid Hypofunction Disorders | n (%) | 3 (<1%) |
| Acquired Hypothyroidism | n (%) | 3 (<1%) |
| Thyroid Disorders NEC | n (%) | 2 (<1%) |
| Goitre | n (%) | 2 (<1%) |
| Thyroid Hyperfunction Disorders | n (%) | 2 (<1%) |
| Thyrotoxicosis | n (%) | 2 (<1%) |
| Acute and Chronic Thyroiditis | n (%) | 1 (<1%) |
| Thyroiditis NOS | n (%) | 1 (<1%) |
| Thyroid Neoplasms | n (%) | 1 (<1%) |
| Thyroid Nodule | n (%) | 1 (<1%) |
| Congenital, Familial and Genetic Disorders | n (%) | 3 (<1%) |
| Skin and Subcutaneous Tissue Disorders Congenital NEC | n (%) | 2 (<1%) |
| Pigmented Naevus | n (%) | 2 (<1%) |
| Musculoskeletal and Connective Tissue Disorders of Limbs | n (%) | 1 (<1%) |
| Congenital | | |
| Congenital Claw Toe | n (%) | 1 (<1%) |
| Social Circumstances | n (%) | 3 (<1%) |
| Age Related Issues | n (%) | 3 (<1%) |
| Menopause* | n (%) | 3 (<1%) |
| Pregnancy, Puerperium and Perinatal Conditions | n (%) | 2 (<1%) |
| Normal Pregnancy, Labour and Delivery* | n (%) | 2 (<1%) |
| Pregnancy NOS* | n (%) | 2 (<1%) |
| Surgical and Medical Procedures | n (%) | 2 (<1%) |
| Bone Therapeutic Procedures NEC | n (%) | 1 (<1%) |
| Osteosynthesis | n (%) | 1 (<1%) |
| Fallopian Tube Therapeutic Procedures* | n (%) | 1 (<1%) |
| Tubal Ligation* | n (%) | 1 (<1%) |

5050
Safety Evaluation

The most common AEs occurring in >5% of subjects in the cilansetron and placebo groups were GI-related. Specific adverse events included: GI atonic and hypomotility disorders (11% v. 6%); GI and abdominal pain (excluding oral and throat) (12% v. 6%); and nausea and vomiting (7% v. 6%). The most common AEs leading to study discontinuation were abdominal pain (2% v. 1%) and constipation (2% v. 1%) in the cilansetron and placebo groups, respectively. All other AEs leading to discontinuation were experienced by no more than one person in the cilansetron group. Incidences of the most common treatment-related AEs are presented in Table 21. The most common treatment-related AEs observed at a greater rate in the cilansetron group than in the placebo group were constipation (8% v. 3%) and abdominal pain not otherwise specified (5% v. 1%). A summary of all AEs is presented in Table 21.

TABLE 21

| | Cilansetron 2 mg (N = 301) | Placebo (N = 94) |
|---|---|---|
| Adverse Event | N (%) | N (%) |
| Constipation | 24 (8) | 3 (3) |
| Nausea | 15 (5) | 4 (4) |
| Abdominal Pain (not | 16 (5) | 1 (1) |
| otherwise specified) | | |
| Flatulence | 6 (2) | 3 (3) |
| Diarrhea (not otherwise | 5 (2) | 3 (3) |
| specified) | | |
| Rectal Hemmorhage | 8 (3) | 1 (1) |
| Alanine aminotransferase | 9 (3) | 2 (2) |
| increased | | |
| Headache (not otherwise | 13 (4) | 4 (4) |
| specified) | | |

What is claimed is:

1. A method of treatment of diarrhea-predominant IBS in a subject, comprising:
administering to a subject in need of treatment a therapeutically effective amount of a composition comprising cilansetron or a pharmaceutically acceptable derivative thereof; and providing information that indicates that constipation, abdominal pain, upper respiratory tract infection and/or nausea may occur after administering the composition.

2. The method of claim 1, wherein the composition comprises about 2 mg of cilansetron.

3. The method of claim 1, wherein the composition is administered three or more times daily.

4. The method of claim 1, wherein the composition comprises:
(i) about 1.5 mg to about 3 mg cilansetron.HCl.H₂0;
(ii) about 40 mg to about 60 mg corn starch;
(iii) about 70 mg to about 100 mg mannitol;
(iv) about 3 mg to about 7 mg povidone;
(v) about 0.05 mg to about 1 mg citric acid monohydrate;
(vi) about 1 mg to about 5 mg crospovidone;
(vii) about 0.05 mg to about 2 mg colloidal silica; and
(viii) about 1 mg to about 3 mg stearic acid.

5. A method of treatment of diarrhea-predominant IBS in a subject, comprising:
administering to a subject in need of treatment a therapeutically effective amount of a composition comprising cilansetron or a pharmaceutically acceptable derivative thereof; and information that indicates that angina pectoris and/or a cardiac arrhythmia may occur after administering the composition.

6. The method of claim 5, wherein the cardiac arrhythmia is selected from ventricular tachycardia and sinus bradycardia.

7. A method of treatment of diarrhea-predominant IBS in a subject, comprising:
administering to a subject in need of treatment a therapeutically effective amount of a composition comprising cilansetron or a pharmaceutically acceptable derivative thereof; and providing to the subject dosage, administration and adverse reaction information pertaining to the composition, wherein the adverse reaction information comprises information that indicates that constipation, abdominal pain, upper respiratory tract infection and/or nausea may occur after administering the composition.

8. A method of treatment of diarrhea-predominant IBS in a subject, comprising:
administering to a subject in need of treatment a therapeutically effective amount of a composition comprising cilansetron or a pharmaceutically acceptable derivative thereof; and providing to the subject dosage, administration and adverse reaction information pertaining to the composition, wherein the adverse reaction information comprises information that indicates that angina pectoris and/or a cardiac arrhythmia may occur after administering the composition.

9. A method of treatment of diarrhea-predominant IBS in a female subject, comprising:
administering to a subject in need of treatment a therapeutically effective amount of a composition comprising cilansetron or a pharmaceutically acceptable derivative thereof; and providing information that indicates that constipation, diarrhea, sinusitis, and/or urinary tract infection may occur after administering the composition.

10. A method of treatment of diarrhea-predominant IBS in a male subject, comprising:
administering to a subject in need of treatment a therapeutically effective amount of a composition comprising cilansetron or a pharmaceutically acceptable derivative thereof; and providing information that indicates that an increase in blood creatinine phosphokinase may occur after administering the composition.

11. A method of treatment of diarrhea-predominant IBS in a subject, comprising:
administering to a subject in need of treatment a therapeutically effective amount of a composition comprising cilansetron or a pharmaceutically acceptable derivative thereof; and monitoring the subject for a treatment-emergent adverse event selected from constipation, abdominal pain, nausea, upper respiratory tract infection, urinary tract infection, rectal hemorrhage, angina pectoris, a cardiac arrhythmia, sinusitis, fecal occult blood, nasopharyngitis, and/or an increase in blood creatinine phosphokinase.

12. The method of claim 11, wherein at least one treatment-emergent adverse event is detected, the dosage of the composition administered is altered to a dosage that does not produce the at least one treatment-emergent adverse event.

13. The method of claim 12, wherein if at least one treatment-emergent adverse event is detected, administration of the composition is ceased.

14. A method for safe long-term treatment of diarrhea-predominant IBS in a subject, comprising administering a composition comprising cilansetron or a pharmaceutically acceptable derivative thereof to a subject in need of treatment for a period of at least about 52 weeks.

15. The method of claim 14, wherein the composition comprises about 2 mg of cilansetron.

16. The method of claim 14, wherein the composition is administered three or more times daily.

17. The method of claim 14, wherein the composition comprises:
(i) about 1.5 mg to about 3 mg cilansetron.HCl.H₂0;
(ii) about 40 mg to about 60 mg corn starch;
(iii) about 70 mg to about 100 mg mannitol;
(iv) about 3 mg to about 7 mg povidone;
(v) about 0.05 mg to about 1 mg citric acid monohydrate;
(vi) about 1 mg to about 5 mg crospovidone;
(vii) about 0.05 mg to about 2 mg colloidal silica; and
(viii) about 1 mg to about 3 mg stearic acid.

18. A method of treatment of diarrhea-predominant IBS in a subject, comprising:
administering a composition comprising cilansetron or a pharmaceutically acceptable derivative thereof to a subject in need of treatment in an amount sufficient to substantially provide relief of at least one symptom associated with diarrhea-predominant IBS in the subject for a period of at least about 52 weeks.

19. A method of improving quality of life in a subject having diarrhea-predominant IBS, comprising:
administering a composition comprising cilansetron or a pharmaceutically acceptable derivative thereof to a subject in need of treatment in an amount sufficient to substantially improve quality of life in the subject for a period of at least about 52 weeks.

20. A method of treatment of diarrhea-predominant IBS in a male subject, comprising:
administering a composition comprising cilansetron or a pharmaceutically acceptable derivative thereof in an amount sufficient to substantially maintain a prevalence of treatment-emergent constipation of less than about 15% across a statistically significant population of male subjects for a period of at least about 52 weeks.
